(19) United States
(12) Patent Application Publication
(10) Pub. No.: US 2024/0076318 A1
(43) Pub. Date: Mar. 7, 2024

(54) EPSTEIN-BARR VIRUS ANTIGEN CONSTRUCTS

(71) Applicant: GLAXOSMITHKLINE BIOLOGICALS SA, Rixensart (BE)

(72) Inventors: Stefania CAPONE, Rome (IT); Antonella FOLGORI, Rome (IT); Armin LAHM, Rome (IT); Benjamin WIZEL, Rockville, MD (US)

(73) Assignee: GLAXOSMITHKLINE BIOLOGICALS SA, Rixensart (BE)

(21) Appl. No.: 18/358,350

(22) Filed: Jul. 25, 2023

Related U.S. Application Data
(63) Continuation of application No. 16/770,963, filed on Jun. 9, 2020, now Pat. No. 11,773,139.
(60) Provisional application No. 62/608,038, filed on Dec. 20, 2017.

(51) Int. Cl. C07K 14/005; A61K 39/245; C12N 7/00

(52) U.S. Cl. CPC C07K 14/005; A61K 39/245; C12N 7/00; A61K 2039/70

(57) ABSTRACT

The invention provides Epstein-Barr Virus antigen polynucleotides, polypeptides and vectors; as well as immunogenic compositions comprising the same. It includes the use of Epstein-Barr Virus antigen constructs to produce vaccines for treating and preventing Epstein-Barr Virus infections and Epstein-Barr Virus-associated diseases, such as multiple sclerosis, rheumatoid arthritis and systemic lupus erythematosus.

### CROSS-REFERENCE TO RELATED APPLICATIONS

This application is a continuation of U.S. application Ser. No. 16/770,963, filed Jun. 9, 2020, which was filed pursuant to 35 U.S.C. § 371 as a United States National Phase Application of International Application No. PCT/162018/060101, filed Dec. 14, 2018, which claims priority from U.S. Provisional Application No. 62/608,038, filed Dec. 20, 2017, the entireties of each of which are hereby incorporated herein by reference.

### STATEMENT OF GOVERNMENT INTEREST

This invention was created in the performance of a Cooperative Research and Development Agreement with the National Institutes of Health, an Agency of the Department of Health and Human Services. The Government of the United States has certain rights in this invention.

### SEQUENCE LISTING

The instant application contains a Sequence Listing which has been submitted electronically in XML file format and is hereby incorporated by reference in its entirety. Said XML copy, created on Jul. 13, 2023, is named VU66487C1-US_SL.xml and is 307,250 bytes in size.

### FIELD OF THE INVENTION

This invention is in the field of treating and preventing viral infections. In particular, the present invention relates to Epstein Barr Virus antigen constructs. It includes the use of Epstein-Barr Virus antigen constructs for treating and preventing Epstein-Barr Virus infections and Epstein-Barr Virus- associated diseases.

### BACKGROUND

Epstein-Barr Virus (EBV), also known as human herpesvirus 4 (HHV-4), is one of the most common viruses in humans, infecting at least 90% of adults. EBV establishes asymptomatic latent infection in most infected individuals, but is also known as the primary causative agent of infectious mononucleosis.

More significantly, EBV infection is associated with certain types of malignancies (e.g., gastric carcinoma, nasopharyngeal carcinoma, Hodgkin's lymphoma, non-Hodgkin's lymphoma, Burkitt's lymphoma) as well as an increased risk of multiple sclerosis (MS), systemic lupus erythematosus (SLE), rheumatoid arthritis (RA) and Sjogren's syndrome.

Like other members of the herpesvirus family, EBV contains a double-stranded DNA genome of about 192 kilobases encoding about 85 genes. The EBV genome is encased in a protein nucleocapsid surrounded by a viral tegument. An outer envelope layer comprises lipids and surface glycoproteins which are thought to be involved in targeting the virus to its primary host cells, B lymphocytes and epithelial cells.

The EBV viral replication cycle is well-characterized. After initial infection of host cells, EBV enters a stage of active production of infectious virions, termed the lytic replication stage (or lytic stage). During the lytic stage, EBV gene expression is characterized by expression of one or more lytic gene products, including ZEBRA, BRLF1, BNLF2, BCRF1, and viral capsid antigens (VCAs), as well as envelope glycoproteins such as gp350 and gp110.

Following a period of lytic replication, EBV enters a state of persistent viral infection without active viral production, termed latency (or the latent phase). Latent EBV infection is accompanied by characteristic gene expression programs, including expression of one or more latent gene products such as EBNA1, EBNA2, EBNA3A, EBNA3B, EBNA3C, EBNA leader protein (EBNA-LP), LMP1, and/or LMP2. Latently-infected cells can be reactivated to lytic viral production by triggers which are not yet understood.

A number of EBV vaccine candidates have been evaluated in animal models and human trials. Most prophylactic vaccine candidates have focused on the major EBV envelope glycoprotein gp350 as the immunogen. Gu et al. reported that a recombinant live vaccinia virus expressing EBV gp350 elicited EBV neutralizing antibodies and modest protection in children, but not in adults. Gu et al., Dev. Biol. Stand. 1995; 84: 171-177. A recombinant gp350 vaccine was found not to protect against EBV infection, but reduced the occurrence of infectious mononucleosis. Sokal et al., J. Infect. Dis. 2007; 196(12):1749-1753.

Therapeutic EBV vaccine candidates have primarily targeted T-cell epitopes of EBV nuclear antigen-1 (EBNA1) and LMP2. For example, Taylor et al. have described a modified vaccinia virus Ankara (MVA) vector expressing a peptide fragment of EBNA1 fused to the full-length LMP2 protein. The so-called MVA-EL vaccine was reported to induce antigen-specific CD4+ and CD8+ T cell responses in early clinical trials. Taylor et al., J. Virol. January 2004, p. 768-778. Similarly, a recombinant human adenoviral vector expressing full length LMP2 protein was reported to induce antigen-specific T-cell responses in vitro and in in mice. Pan et al., Biochem Biophys Res Commun. 2006 Sep. 1; 347(3):551-7.

Despite the clear need in the art, no EBV vaccine has yet been licensed for use in humans. Thus, there remains a need for an EBV vaccine for use in preventing EBV infection as well as in treating EBV-associated malignancies and EBV-associated diseases, such as multiple sclerosis.

### SUMMARY OF THE INVENTION

The present inventors provide EBV antigen polypeptides, polynucleotides and vectors useful as components of immunogenic compositions for the induction of an immune response in a subject against Epstein-Barr Virus (EBV) infection; methods for their use in prevention and treatment of EBV infection and EBV-associated diseases; and processes for their manufacture.

There is provided a polynucleotide encoding an EBV antigen polypeptide comprising:
(a) at least one fragment of at least 8 amino acids of SEQ ID NO: 1,
(b) at least one fragment of at least 8 amino acids of SEQ ID NO: 6,
(c) at least one fragment of at least 8 amino acids of SEQ ID NO: 11, and
(d) at least one fragment of at least 8 amino acids of SEQ ID NO: 13; wherein the polynucleotide is operatively linked to one or more sequences which direct expression of said polypeptide in a host cell. In some embodiments, the polypeptide further comprises at least one fragment of at least 8 amino acids of SEQ ID NO: 21.

Also provided is a polynucleotide encoding an EBV antigen polypeptide comprising:
(a) at least two LMP1 fragments of at least 8 amino acids of SEQ ID NO: 1, wherein the LMP1 fragments are not adjacent to each other,
(b) at least two LMP2 fragments of at least 8 amino acids of SEQ ID NO: 6, wherein the LMP2 fragments are not adjacent to each other,
(c) at least two EBNA1 fragments of at least 8 amino acids of SEQ ID NO: 11, wherein the EBNA1 fragments are not adjacent to each other,
(d) at least two EBNA3A fragments of at least 8 amino acids of SEQ ID NO: 13, wherein the EBNA3A fragments are not adjacent to each other, and/or
(e) at least two ZEBRA fragments of at least 8 amino acids of SEQ ID NO: 21, wherein the ZEBRA fragments are not adjacent to each other;
   wherein the polynucleotide is operatively linked to one or more sequences which direct expression of said polypeptide in a host cell.

Also provided is a polynucleotide as described above, wherein the EBV antigen polypeptide comprises:
(a) a first fragment of LMP1 consisting of SEQ ID NO: 2,
(b) a second fragment of LMP1 consisting of SEQ ID NO: 3,
(c) a third fragment of LMP1 consisting of SEQ ID NO: 4,
(d) a fourth fragment of LMP1 consisting of SEQ ID NO: 5,
(e) a first fragment of LMP2 consisting of SEQ ID NO: 7,
(f) a second fragment of LMP2 consisting of SEQ ID NO: 8,
(g) a third fragment of LMP2 consisting of SEQ ID NO: 9,
(h) a fourth fragment of LMP2 consisting of SEQ ID NO: 10,
(i) a first fragment of EBNA1 consisting of SEQ ID NO: 12,
(j) a first fragment of EBNA3A consisting of SEQ ID NO: 14,
(k) a second fragment of EBNA3A consisting of SEQ ID NO: 15,
(l) a third fragment of EBNA3A consisting of SEQ ID NO: 16,
(m) a fourth fragment of EBNA3A consisting of SEQ ID NO: 17,
(n) a fifth fragment of EBNA3A consisting of SEQ ID NO: 18,
(o) a sixth fragment of EBNA3A consisting of SEQ ID NO: 19, and
(p) a seventh fragment of EBNA3A consisting of SEQ ID NO: 20;
   wherein the first, second, third and fourth LMP1 fragments are not adjacent to each other; the first, second, third and fourth LMP2 fragments are not adjacent to each other; and the first, second, third, fourth, fifth, sixth, and seventh EBNA3A fragments are not adjacent to each other. Optionally, the polypeptide further comprises: (a) a first fragment of ZEBRA consisting of SEQ ID NO: 22, and (b) a second fragment of ZEBRA consisting of SEQ ID NO: 23; wherein the first and second ZEBRA fragments are not adjacent to each other.

Also provided is a polynucleotide as described above, wherein the EBV antigen polypeptide is at least 80% identical to SEQ ID NO: 24 or SEQ ID NO: 26.

Also provided are vectors comprising the polynucleotides as described herein, including, for example, adenovirus vectors (e.g., non-human simian adenovirus vectors) and vaccinia virus vectors (e.g., modified vaccinia Ankara (MVA) vectors).

Also provided are EBV antigen polypeptides encoded by the polynucleotides and vectors as described herein, such as polypeptides at least 80% identical to SEQ ID NO: 24 or SEQ ID NO: 26.

Also provided are compositions comprising polynucleotides, vectors and polypeptides described herein; and a pharmaceutically acceptable excipient. Such compositions optionally comprise one or more adjuvants.

Also provided are uses of the polynucleotides, vectors, polypeptides and compositions as described herein, in the manufacture of a medicament for the treatment or prophylaxis of a disease caused by Epstein-Barr Virus infection.

Also described are methods of inducing an immune response in a subject comprising administering the polynucleotides, vectors, polypeptides and compositions as described herein to the subject.

Also provided are methods of treating or preventing an EBV-associated disease in a subject, comprising administering the polynucleotides, vectors, polypeptides and compositions as described herein to the subject. EBV-associated diseases include, for example, EBV-associated diseases (e.g., multiple sclerosis, rheumatoid arthritis and systemic lupus erythematosus).

Also provided are the polynucleotides, vectors, polypeptides and compositions as described herein, for use in the treatment or prophylaxis of a disease caused by Epstein-Barr Virus infection.

Also provided are methods of inducing an immune response in a subject comprising:
(a) administering an adenovirus comprising a polynucleotide encoding an
EBV antigen polypeptide as described herein, and
(b) administering a vaccinia virus comprising a polynucleotide encoding an
EBV antigen polypeptide as described herein;
   wherein steps (a) and (b) are conducted in either order.

Also provided is a method of treating or preventing an EBV-associated disease in a subject, comprising:
(a) administering an adenovirus comprising a polynucleotide encoding an
EBV antigen polypeptide as described herein, and
(b) administering a vaccinia virus comprising a polynucleotide encoding an
EBV antigen polypeptide as described herein;
   wherein steps (a) and (b) are conducted in either order.

### DESCRIPTION OF THE DRAWINGS

FIG. 1: Schematic representation of EBV latent (LMP1, LMP2, EBNA1 and EBNA3A) and lytic (ZEBRA) proteins. Numbered regions indicate immunogenic fragments used to construct polyvalent EBV antigen polypeptides. Alphanumeric identifiers (e.g., a1, a2, b1, b2) correspond to the fragments represented in polyvalent constructs shown in FIGS. 2A and 2B.

FIG. 2A-B: Schematic representation of polyvalent EBV antigen constructs. FIG. 2A shows an EBV latent antigen construct (EBV-L), comprising immunogenic fragments derived from LMP2 (identified as a1 to a4), LMP1 (identified as b1 to b4), EBNA1 (identified as c1) and EBNA3A (identified as d1 to d7). FIG. 2B shows an EBV latent+lytic antigen construct (EBV-LLy), comprising immunogenic fragments derived from LMP1, LMP2, EBNA1, EBNA3A (all identified as in FIG. 2A) and ZEBRA (identified as e1 to e2). Regions of the polyvalent antigens with the same letter prefix (i.e., a, b, c, d, e) are derived from the same EBV protein.

FIG. 3: Schematic representation of CalHV3 latent (C1, C7 and ORF39) and lytic (ORF43) proteins. Numbered regions indicate immunogenic fragments used to construct polyvalent CalHV3 antigen polypeptides. Alphanumeric identifiers (e.g., a1, a2, b1, b2) correspond to the fragments represented in polyvalent constructs shown in FIG. 4A-C. C1, C7, ORF39 and ORF43 antigens were selected because they are the putative marmoset (Callithrix jacchus) herpesvirus orthologs of EBV proteins LMP1, LMP2, EBNA1 and ZEBRA, respectively.

FIG. 4A-C: Schematic representation of polyvalent CalHV3 antigen constructs.

FIG. 4A shows a CalHV3 latent antigen construct (CalHV3-L), comprising immunogenic fragments derived from C1, C7 and ORF39. FIG. 4B shows a CalHV3 latent+lytic antigen construct (CalHV3-LLy), comprising immunogenic fragments derived from C1, C7, ORF39 and ORF43. FIG. 4C shows a CalHV3-LLy construct from FIG. 4B, fused to the marmoset MHC class II-associated invariant chain (Ii). Regions of the polyvalent antigens with the same letter prefix (i.e., a, b, c, d) are derived from the same CalHV3 protein.

FIG. 5A: T-cell responses in mice immunized with adenoviral vectors encoding EBV antigen constructs at doses of 10⁶, 10⁷ and 10⁸ viral particles (vp). ChAd155-EBV-L and ChAd155-EBV-LLy elicited interferon gamma (IFNγ) secretion from splenocytes in a dose-dependent manner. T-cell activation in response to stimulation with peptide pools covering LMP1, LMP2, EBNA1 and EBNA3A was observed to each of the EBV latent antigens in both EBV-L and EBV-LLy immunized mice. T-cell responses to the EBV lytic protein, ZEBRA, were only detected in EBV-LLy immunized mice. No responses were detected to the EBV junctional peptides (Ej) or to DMSO (dimethyl sulfoxide), the peptide pool diluent, used as a negative control.

FIG. 5B: T-cell responses in mice immunized with adenoviral vectors encoding CalHV3 antigen constructs at doses of 10⁶, 10⁷ and 10⁸ vp. ChAd155-CalHV3-L and ChAd155-CalHV3-LLy elicited IFNγ secretion from splenocytes in a dose-dependent manner. T-cell responses to peptide pools covering CalHV3 latent antigens C1, C7 and ORF39 were detected in both CalHV3-L and CalHV3-LLy immunized mice. However, T-cell responses to the CalHV3 lytic protein, ORF43, were only detected in CalHV3-LLy immunized mice. No responses were detected to the CalHV3 junctional peptides (Cj) or the negative control, DMSO.

FIG. 6A-B: Effect of prime-boost dosing on EBV-LLy immunogenicity in mice. Immunization with ChAd155-EBV-LLy on day 0 followed by a boost immunization with MVA-EBV-LLy on day 21 (week 3) produced a significant increase in EBV-specific interferon gamma release, as compared to unboosted mice or mice receiving a “boost” injection of an MVA vector encoding an EBV-unrelated antigen. FIG. 6A presents the cumulative T-cell responses to all antigens (LMP1, LMP2, EBNA1, EBNA3A and ZEBRA), and FIG. 6B shows the responses to individual antigens and to the negative control, DMSO.

FIG. 7: Effect of prime-boost dosing on CalHV3-LLy immunogenicity in mice. Immunization with ChAd155-CalHV3-LLy (“prime”) on day 0 followed by a boost immunization with either the same antigen construct (ChAd155-CalHV3-LLy) or MVA-CalHV3-LLy on day 42 (week 6) produced a significant increase in CalHV3-specific IFNγ release measured at week 7, as compared to unboosted mice or mice receiving a “boost” injection of an MVA vector encoding a CalHV3-unrelated antigen.

FIG. 8: Cumulative T-cell responses in mice two weeks after immunization with an invariant chain-CalHV3-LLy fusion protein (ChAd155-li-CalHV3-LLy). At the lower dose of antigen (5×10⁶ vp), ChAd155-li-CalHV3-LLy elicited significantly greater IFNγ release and a higher proportion (100%) of responding mice than ChAd155-CalHV3-LLy. No differences were observed at the higher antigen dose tested (5×10⁷ vp).

FIG. 9: Cumulative T-cell responses in CalHV3-infected marmosets. Prior to immunization (w0), animals exhibited baseline CalHV3-specific T-cell responses, consistent with the fact that the animals are virus carriers. Three weeks after ChAd155-CalHV3-LLy immunization (w3 pp), CalHV3-immunized animals exhibited significant expansion of pre-existing CalHV3-specific T cell responses. T-cell responses continued to be elevated above baseline 1 week post-boost (w1 pb) with MVA-CalHV3-LLy, and after contraction were still above baseline 7 weeks after boost.

FIG. 10: The enhanced cumulative T-cell response to ChAd155-CalHV3-LLy shown in FIG. 9 was determined to be polyspecific, i.e., against CalHV3-LLy antigens C1, C7, ORF39 and ORF 43.

FIG. 11: EBV peptide pools corresponding to antigens encoded by ChAd155 and MVA latent+lytic EBV vaccines elicit IFNγ release in PBMCs from human healthy donors.

### DESCRIPTION OF THE SEQUENCES

SEQ ID NO: 1 EBV LMP1 protein (Genbank No. P03230)
SEQ ID NO: 2 immunogenic fragment of EBV LMP1 protein
SEQ ID NO: 3 immunogenic fragment of EBV LMP1 protein
SEQ ID NO: 4 immunogenic fragment of EBV LMP1 protein
SEQ ID NO: 5 immunogenic fragment of EBV LMP1 protein
SEQ ID NO: 6 EBV LMP2 protein (Genbank No. P13285)
SEQ ID NO: 7 immunogenic fragment of EBV LMP2 protein
SEQ ID NO: 8 immunogenic fragment of EBV LMP2 protein
SEQ ID NO: 9 immunogenic fragment of EBV LMP2 protein
SEQ ID NO: 10 immunogenic fragment of EBV LMP2 protein
SEQ ID NO: 11 EBV EBNA1 protein (Genbank No. P03211)
SEQ ID NO: 12 immunogenic fragment of EBV EBNA1 protein
SEQ ID NO: 13 EBV EBNA3A protein (Genbank No. YP401669)
SEQ ID NO: 14 immunogenic fragment of EBV EBNA3A protein
SEQ ID NO: 15 immunogenic fragment of EBV EBNA3A protein
SEQ ID NO: 16 immunogenic fragment of EBV EBNA3A protein
SEQ ID NO: 17 immunogenic fragment of EBV EBNA3A protein
SEQ ID NO: 18 immunogenic fragment of EBV EBNA3A protein
SEQ ID NO: 19 immunogenic fragment of EBV EBNA3A protein
SEQ ID NO: 20 immunogenic fragment of EBV EBNA3A protein
SEQ ID NO: 21 EBV ZEBRA protein (Genbank No. P03206)
SEQ ID NO: 22 immunogenic fragment of EBV ZEBRA protein
SEQ ID NO: 23 immunogenic fragment of EBV ZEBRA protein
SEQ ID NO: 24 EBV-L antigen polypeptide
SEQ ID NO: 25 DNA encoding EBV-L antigen polypeptide
SEQ ID NO: 26 EBV-LLy antigen polypeptide
SEQ ID NO: 27 DNA encoding EBV-LLy antigen polypeptide
SEQ ID NO: 28 CalHV3 C1 protein (Genbank No. NP_733852)
SEQ ID NO: 29 immunogenic fragment of CalHV3 C1 protein
SEQ ID NO: 30 immunogenic fragment of CalHV3 C1 protein
SEQ ID NO: 31 immunogenic fragment of CalHV3 C1 protein
SEQ ID NO: 32 CalHV3 C7 protein (Genbank No. NP_733851)
SEQ ID NO: 33 immunogenic fragment of CalHV3 C7 protein
SEQ ID NO: 34 immunogenic fragment of CalHV3 C7 protein
SEQ ID NO: 35 immunogenic fragment of CalHV3 C7 protein
SEQ ID NO: 36 CalHV3 ORF39 (Genbank No. NP_733892)
SEQ ID NO: 37 immunogenic fragment of CalHV3 ORF39
SEQ ID NO: 38 immunogenic fragment of CalHV3 ORF39
SEQ ID NO: 39 immunogenic fragment of CalHV3 ORF39
SEQ ID NO: 40 CalHv3 ORF43 protein (Genbank No. NP_733896)
SEQ ID NO: 41 immunogenic fragment of CalHv3 ORF43 protein
SEQ ID NO: 42 immunogenic fragment of CalHv3 ORF43 protein
SEQ ID NO: 43 marmoset invariant chain polypeptide
SEQ ID NO: 44 CalHv3_L antigen polypeptide
SEQ ID NO: 45 DNA encoding CalHV3_L antigen polypeptide
SEQ ID NO: 46 CalHV3_LLy antigen polypeptide
SEQ ID NO: 47 DNA encoding CalHV3_LLy antigen polypeptide
SEQ ID NO: 48 li_CalHV3_LLy antigen polypeptide
SEQ ID NO: 49 DNA encoding li_CalHV3_LLy antigen polypeptide
SEQ ID NO: 50 pChAd155 (ΔE1, ΔE4_Ad5E4 orf6) TetO hCMV-EBV-L expression vector
SEQ ID NO: 51 pChAd155 (ΔE1, ΔE3, ΔE4_Ad5E4 orf6) TetO hCMV-EBV-LLy expression vector
SEQ ID NO: 52 pChAd155 (ΔE1, ΔE4_Ad5E4 orf6) TetO hCMV-CalHV3-L expression vector
SEQ ID NO: 53 pChAd155 (ΔE1, ΔE3, ΔE4_Ad5E4 orf6) TetO hCMV-CalHV3-LLy expression vector
SEQ ID NO: 54 pChAd155 (ΔE1, ΔE3, ΔE4_Ad5E4 orf6) TetO hCMV-mli-CalHV3-LLy expression vector

### DETAILED DESCRIPTION OF THE INVENTION

### Epstein-Barr Virus Antigen Polypeptides

EBV antigen polypeptides of the invention include polypeptides comprising immunogenic fragments of one or more EBV latent and/or lytic proteins. EBV latent proteins include, for example, Latent Membrane Proteins (LMP1 and LMP2); and EBV Nuclear Antigens (EBNA1, EBNA2, EBNA3A, EBNA3B and EBNA3C). EBV lytic proteins include, for example, ZEBRA (encoded by the BZLF1 gene).

An “immunogenic fragment” of an EBV protein, as used herein, means a fragment smaller than a full-length EBV protein that is capable of inducing an immune response, for example a humoral (e.g., antibody) and/or cell-mediated (e.g., a cytotoxic T cell) response. Immunogenic fragments include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of the full length protein. In some embodiments, immunogenic fragments consist of about 8, about 9, about 10, about 11, about 12, about 13, about 14, about 15, about 20, about 30, about 40, about 50, about 60, about 70, about 80, about 90 and about 100 amino acids of the full length protein. One aspect of the invention is to provide EBV antigen polypeptides capable of inducing T-cell responses against B-cells harboring latent EBV infection. Thus, in some embodiments, immunogenic fragments of EBV proteins comprise one or more T-cell epitopes capable of inducing an antigen-specific T-cell response.

Immunogenic fragments may have one or more substitutions, deletions or insertions relative to the full length protein from which the fragment is derived. Thus, immunogenic fragments include fragments at least 80%, at least 85%, at least 90%, at least 95% or at least 99% identical to the corresponding region of the full length protein.

In one embodiment, an EBV antigen polypeptide of the invention comprises a Latent Membrane Protein 1 (LMP1) antigen. LMP1 is a 386 amino acid protein expressed during the latent stage of the EBV viral life cycle. Immunogenic fragments of LMP1 suitable for use in the EBV antigen polypeptides of the invention include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of SEQ ID NO: 1. In some embodiments, immunogenic fragments of LMP1 consist of about 8, about 9, about 10, about 11, about 12, about 13, about 14, about 15, about 20, about 30, about 40, about 50, about 60, about 70, about 80, about 90 and about 100 amino acids of SEQ ID NO: 1. In some embodiments, immunogenic fragments of LMP1 include fragments at least 80%, at least 85%, at least 90%, at least 95% or at least 99% identical to the corresponding region of SEQ ID NO: 1.

In some embodiments, an immunogenic fragment of LMP1 comprises one or more T-cell epitopes. In preferred embodiments, immunogenic epitopes of LMP1 include, but are not limited to, SEQ ID Nos: 2-5 and fragments at least 80%, at least 85%, at least 90%, at least 95% or at least 99% identical to SEQ ID Nos: 2-5.

In one embodiment, an EBV antigen polypeptide of the invention comprises a Latent Membrane Protein 2 (LMP2) antigen. LMP2 is a 497 amino acid protein expressed during the latent stage of the EBV viral life cycle. Immunogenic fragments of LMP2 suitable for use in the EBV antigen polypeptides of the invention include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of SEQ ID NO: 6. In some embodiments, immunogenic fragments of LMP2 consist of about 8, about 9, about 10, about 11, about 12, about 13, about 14, about 15, about 20, about 30, about 40, about 50, about 60, about 70, about 80, about 90 and about 100 amino acids of SEQ ID NO: 6. In some embodiments, immunogenic fragments of LMP2 include fragments at least 80%, at least 85%, at least 90%, at least 95% or at least 99% identical to the corresponding region of SEQ ID NO: 6.

In some embodiments, an immunogenic fragment of LMP2 comprises one or more T-cell epitopes. In preferred embodiments, immunogenic epitopes of LMP2 include, but are not limited to, SEQ ID Nos: 7-10, and fragments at least 80%, at least 85%, at least 90%, at least 95% or at least 99% identical to SEQ ID Nos: 7-10.

In one embodiment, an EBV antigen polypeptide of the invention comprises an Epstein-Barr Nuclear Antigen 1 (EBNA1) antigen. EBNA1 is a 641 amino acid protein expressed during the latent stage of the EBV viral life cycle. Immunogenic fragments of EBNA1 suitable for use in the EBV antigen polypeptides of the invention include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of SEQ ID NO: 11. In some embodiments, immunogenic fragments of EBNA1 consist of about 8, about 9, about 10, about 11, about 12, about 13, about 14, about 15, about 20, about 30, about 40, about 50, about 60, about 70, about 80, about 90 and about 100 amino acids of SEQ ID NO: 11. In some embodiments, immunogenic fragments of EBNA1 include fragments at least 80%, at least 85%, at least 90%, at least 95% or at least 99% identical to the corresponding region of SEQ ID NO: 11.

In some embodiments, an immunogenic fragment of EBNA1 comprises one or more T-cell epitopes. In preferred embodiments, immunogenic epitopes of EBNA1 include, but are not limited to, SEQ ID No: 12, and fragments at least 80%, at least 85%, at least 90%, at least 95% or at least 99% identical to SEQ ID No: 12..

In one embodiment, an EBV antigen polypeptide of the invention comprises an Epstein-Barr Nuclear Antigen 3A (EBNA3A) antigen. EBNA3A is a 944 amino acid protein expressed during the latent stage of the EBV viral life cycle. Immunogenic fragments of EBNA3A suitable for use in the EBV antigen polypeptides of the invention include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of SEQ ID NO: 13. In some embodiments, immunogenic fragments of EBNA3A consist of about 8, about 9, about 10, about 11, about 12, about 13, about 14, about 15, about 20, about 30, about 40, about 50, about 60, about 70, about 80, about 90 and about 100 amino acids of SEQ ID NO: 13. In some embodiments, immunogenic fragments of EBNA3A include fragments at least 80%, at least 85%, at least 90%, at least 95% or at least 99% identical to the corresponding region of SEQ ID NO: 13.

In some embodiments, an immunogenic fragment of EBNA3A comprises one or more T-cell epitopes. In preferred embodiments, immunogenic epitopes of EBNA3A include, but are not limited to, SEQ ID Nos: 14-20, and fragments at least 80%, at least 85%, at least 90%, at least 95% or at least 99% identical to SEQ ID Nos: 14-20.

In one embodiment, an EBV antigen polypeptide of the invention comprises a ZEBRA antigen. ZEBRA is a 245 amino acid protein expressed during the lytic stage of the EBV viral life cycle. Immunogenic fragments of ZEBRA suitable for use in the EBV antigen polypeptides of the invention include fragments of at least 8, at least 9, at least 10, at least 11, at least 12, at least 13, at least 14, at least 15, at least 20, at least 30, at least 40, at least 50, at least 60, at least 70, at least 80, at least 90 and at least 100 amino acids of SEQ ID NO: 21. In some embodiments, immunogenic fragments of ZEBRA consist of about 8, about 9, about 10, about 11, about 12, about 13, about 14, about 15, about 20, about 30, about 40, about 50, about 60, about 70, about 80, about 90 and about 100 amino acids of SEQ ID NO: 21. In some embodiments, immunogenic fragments of ZEBRA include fragments at least 80%, at least 85%, at least 90%, at least 95% or at least 99% identical to the corresponding region of SEQ ID NO: 21.

In some embodiments, an immunogenic fragment of ZEBRA comprises one or more T-cell epitopes. In preferred embodiments, immunogenic epitopes of ZEBRA include, but are not limited to, SEQ ID Nos: 22-23, and fragments at least 80%, at least 85%, at least 90%, at least 95% or at least 99% identical to SEQ ID Nos: 22-23.

In some embodiments, an EBV antigen polypeptide is a polyvalent EBV antigen polypeptide. By “polyvalent” is intended a polypeptide comprising immunogenic fragments of two, three, four, five or more EBV proteins. By “fragment” is intended a fragment of at least 8, at least 9, at least 10, at least 11, at least 12, at least 13, at least 14, at least 15, at least 20, at least 30, at least 40, at least 50, at least 60, at least 70, at least 80, at least 90 or at least 100 amino acids of the full-length protein.

Thus, in one embodiment is provided a polypeptide comprising:
(a) at least one fragment of least 8, at least 9, at least 10, at least 11, at least 12, at least 13, at least 14, at least 15, at least 20, at least 30, at least 40, at least 50, at least 60, at least 70, at least 80, at least 90 or at least 100 10 amino acids of SEQ ID NO: 1,
(b) at least one fragment of least 8, at least 9, at least 10, at least 11, at least 12, at least 13, at least 14, at least 15, at least 20, at least 30, at least 40, at least 50, at least 60, at least 70, at least 80, at least 90 or at least 100 amino acids of SEQ ID NO: 6,
(c) at least one fragment of least 8, at least 9, at least 10, at least 11, at least 12, at least 13, at least 14, at least 15, at least 20, at least 30, at least 40, at least 50, at least 60, at least 70, at least 80, at least 90 or at least 100 amino acids of SEQ ID NO: 11, and
(d) at least one fragment of least 8, at least 9, at least 10, at least 11, at least 12, at least 13, at least 14, at least 15, at least 20, at least 30, at least 40, at least 50, at least 60, at least 70, at least 80, at least 90 or at least 100 amino acids of SEQ ID NO: 13.

Optionally, the polypeptide further comprises at least one fragment of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In some embodiments, a polyvalent EBV antigen polypeptide comprises at least two, at least three, at least four, at least five, at least six, at least seven, at least eight, at least nine, or at least ten fragments of one or more EBV proteins. Thus, in one embodiment, a polyvalent EBV antigen is a polypeptide comprising:
(a) at least two fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least two fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least two fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least two fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least two fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least three fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least three fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least three fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least three fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least three fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least four fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least four fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least four fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least four fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least four fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least five fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least five fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least five fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least five fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least five fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least six fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least six fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least six fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least six fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least six fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least seven fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least seven fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least seven fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least seven fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least seven fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least eight fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least eight fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least eight fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least eight fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least eight fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least nine fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least nine fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least nine fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least nine fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least nine fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In one embodiment, a polyvalent EBV antigen is a polypeptide comprising:
(a) at least ten fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 1,
(b) at least ten fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 6,
(c) at least ten fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11,
(d) at least ten fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 13, or
(e) at least ten fragments of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 21.

In some embodiments, the polyvalent EBV antigen polypeptide comprises at least two immunogenic fragments derived from the same EBV protein, wherein the at least two immunogenic fragments are not adjacent to each other in the polyvalent EBV antigen polypeptide. By “not adjacent” is intended to mean that the at least two immunogenic fragments do not form a contiguous amino acid sequence in the EBV antigen polypeptide. Immunogenic fragments which are not adjacent are separated from each other by at least one, two, three, four, five, ten or more amino acids that are not from the same EBV protein as the immunogenic fragments.

For example, in one embodiment, the polyvalent EBV antigen polypeptide comprises at least two, at least three, at least four, at least five, at least six, at least seven, at least eight, at least nine, or at least ten fragments of LMP1 (SEQ ID NO: 1), wherein the fragments of LMP1 are not adjacent to each other.

In another embodiment, the polyvalent EBV antigen polypeptide comprises at least two, at least three, at least four, at least five, at least six, at least seven, at least eight, at least nine, or at least ten fragments of LMP2 (SEQ ID NO: 6), wherein the fragments of LMP2 are not adjacent to each other.

In another embodiment, the polyvalent EBV antigen polypeptide comprises at least two, at least three, at least four, at least five, at least six, at least seven, at least eight, at least nine, or at least ten fragments of EBNA1 (SEQ ID NO: 11), wherein the fragments of EBNA1 are not adjacent to each other.

In another embodiment, the polyvalent EBV antigen polypeptide comprises at least two, at least three, at least four, at least five, at least six, at least seven, at least eight, at least nine, or at least ten fragments of EBNA3A (SEQ ID NO: 13), wherein the fragments of EBNA3A are not adjacent to each other.

In another embodiment, the polyvalent EBV antigen polypeptide comprises at least two, at least three, at least four, at least five, at least six, at least seven, at least eight, at least nine, or at least ten fragments of ZEBRA (SEQ ID NO: 21), wherein the fragments of ZEBRA are not adjacent to each other.

In one embodiment, the polyvalent EBV antigen polypeptide comprises:
(a) a first and a second fragment of LMP1, wherein said first and second fragments of LMP1 are selected from the group consisting SEQ ID NOs: 2-5 and wherein said first and second fragments of LMP1 are not adjacent to each other in the polypeptide,
(b) a first and a second fragment of LMP2, wherein said first and second fragments of LMP1 are selected from the group consisting SEQ ID NOs: 7-10 and wherein said first and second fragments of LMP2 are not adjacent to each other in the polypeptide,
(c) a fragment of EBNA1 consisting of SEQ ID NO: 12, and
(d) a first and a second fragment of EBNA3A, wherein said first and second fragments of EBNA3A are selected from the group consisting SEQ ID NOs: 14-20 and wherein said first and second fragments of EBNA3A are not adjacent to each other in the polypeptide.

Optionally, the polyvalent EBV antigen further comprises:
(a) a first fragment of ZEBRA consisting of SEQ ID NO: 22, and
(b) a second fragment of ZEBRA consisting of SEQ ID NO: 23;
wherein the first and second ZEBRA fragments are not adjacent to each other.

In one embodiment, the polyvalent EBV antigen polypeptide comprises:
(a) a first fragment of LMP1 consisting of SEQ ID NO: 2,
(b) a second fragment of LMP1 consisting of SEQ ID NO: 3,
(c) a third fragment of LMP1 consisting of SEQ ID NO: 4,
(d) a fourth fragment of LMP1 consisting of SEQ ID NO: 5,
(e) a first fragment of LMP2 consisting of SEQ ID NO: 7,
(f) a second fragment of LMP2 consisting of SEQ ID NO: 8,
(g) a third fragment of LMP2 consisting of SEQ ID NO: 9,
(h) a fourth fragment of LMP2 consisting of SEQ ID NO: 10,
(i) a first fragment of EBNA1 consisting of SEQ ID NO: 12,
(j) a first fragment of EBNA3A consisting of SEQ ID NO: 14,
(k) a second fragment of EBNA3A consisting of SEQ ID NO: 15,
(l) a third fragment of EBNA3A consisting of SEQ ID NO: 16,
(m) a fourth fragment of EBNA3A consisting of SEQ ID NO: 17,
(n) a fifth fragment of EBNA3A consisting of SEQ ID NO: 18,
(o) a sixth fragment of EBNA3A consisting of SEQ ID NO: 19, and
(p) a seventh fragment of EBNA3A consisting of SEQ ID NO: 20;
   wherein the first, second, third and fourth LMP1 fragments are not adjacent to each other; the first, second, third and fourth LMP2 fragments are not adjacent to each other; and the first, second, third, fourth, fifth, sixth, and seventh EBNA3A fragments are not adjacent to each other. Optionally, the polyvalent EBV antigen further comprises:
(a) a first fragment of ZEBRA consisting of SEQ ID NO: 22, and
(b) a second fragment of ZEBRA consisting of SEQ ID NO: 23;
   wherein the first and second ZEBRA fragments are not adjacent to each other.

To facilitate a clear description of the polypeptides and polynucleotides described herein, particular sequence components are referred to as a “first” polypeptide or polynucleotide sequence, a “second” polypeptide or polynucleotide sequence, etc. It is to be understood that the first, second, etc. sequences can appear in any desired order or orientation, and that no particular order or orientation is intended by the words “first”, “second” etc.

In some embodiments, the polyvalent EBV antigen does not contain junctional neo-epitopes that map to human (i.e. self) proteins. An immunogenic junctional neo-epitope is an epitope that elicits an immune response to the junction of two heterologous protein sequences, wherein the epitope is not present in either of the heterologous protein sequences themselves. T cell responses to junctional neo-epitopes can be identified using methods known in the art, for example immunological assays using peptide pools covering all junctions to be used, as described in Example 4.

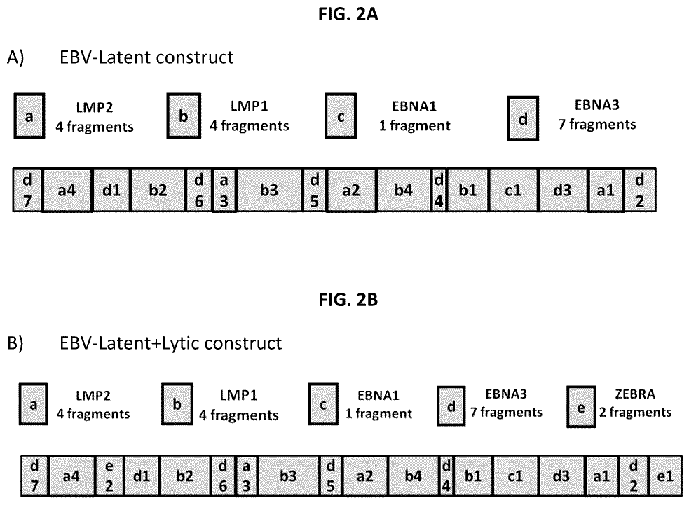

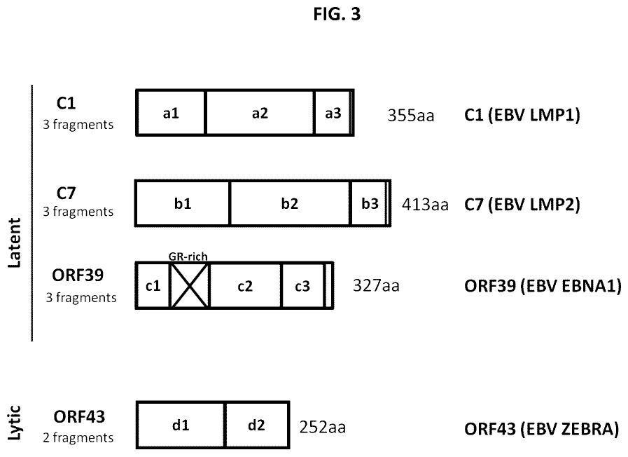

In one embodiment, the polyvalent EBV antigen is the “EBV-L” construct illustrated in FIG. 2A. In another embodiment, the polyvalent EBV antigen polypeptide is a polypeptide at least 80%, at least 85%, at least 90%, at least 95% or at least 99% identical to SEQ ID NO: 24.

In another embodiment, the polyvalent EBV antigen is the “EBV-LLy” construct illustrated in FIG. 2B. In another embodiment, the polyvalent EBV antigen polypeptide is a polypeptide at least 80%, at least 85%, at least 90%, at least 95% or at least 99% identical to SEQ ID NO: 26.

In preferred embodiments, an EBV antigen polypeptide of the invention contains at least one amino acid insertion, deletion and/or substitution as compared to a wild type EBV protein.

In another embodiment, the EBV antigen polypeptide is a polypeptide encoded by a polynucleotide described herein.

### Polynucleotides

Polynucleotides and expression cassettes encoding EBV antigen polypeptides described herein are also provided. By “expression cassette” is meant the combination of a selected heterologous gene (a “transgene” encoding an EBV antigen polypeptide) and the other regulatory elements necessary to drive translation, transcription and/or expression of the gene product in a host cell.

The invention provides a polynucleotide encoding an EBV antigen polypeptide of the invention.

In one embodiment is provided a polynucleotide encoding a polypeptide at least 80%, at least 85%, at least 90%, at least 95% or at least 99% identical to SEQ ID NO: 24. In one embodiment, the polynucleotide is at least 80%, at least 85%, at least 90%, at least 95% or at least 99% identical to SEQ ID NO: 25.

In one embodiment is provided a polynucleotide encoding a polypeptide at least 80%, at least 85%, at least 90%, at least 95% or at least 99% identical to SEQ ID NO: 26. In one embodiment, the polynucleotide is at least 80%, at least 85%, at least 90%, at least 95% or at least 99% identical to SEQ ID NO: 27.

Suitably the polynucleotides of the invention are recombinant. Recombinant means that the polynucleotide is the product of at least one of cloning, restriction, recombination or ligation steps, or other procedures that result in a polynucleotide that is distinct from a polynucleotide found in nature. A recombinant virus is a virus comprising a recombinant polynucleotide. A recombinant vector is a vector comprising a recombinant polynucleotide. A recombinant virus includes progeny of the original recombinant virus. A “recombinant vector” includes replicates of the original recombinant vector. A “recombinant polynucleotide” includes replicates of the original recombinant polynucleotide. Recombinant polynucleotides of the invention contain at least one nucleic acid substitution as compared to the wild-type EBV genome.

In some embodiments, EBV antigen-encoding polynucleotides of the invention are operatively linked to one or more control elements in a manner that permits its transcription, translation and/or expression in a cell transfected or infected with the polynucleotide. As used herein, “operably linked” sequences include both expression control sequences that are contiguous with the gene of interest and expression control sequences that act in trans or at a distance to control the gene of interest. Thus, in one embodiment a polynucleotide is operatively linked to one or more sequences which direct expression of said polypeptide in a host cell. In some embodiments the expression control sequence is heterologous to the EBV antigen-encoding polynucleotide

Expression control sequences include appropriate transcription initiation, termination, promoter and enhancer sequences; efficient RNA processing signals such as splicing and polyadenylation (poly A) signals including rabbit beta-globin polyA; sequences that stabilize cytoplasmic mRNA; sequences that enhance translation efficiency (e.g., Kozak consensus sequence); sequences that enhance protein stability; and when desired, sequences that enhance secretion of the encoded product. Among other sequences, chimeric introns may be used.

A “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A great number of expression control sequences, including promoters which are internal, heterologous, native, constitutive, inducible and/or tissue-specific, are known in the art and may be utilized.

In one embodiment, the polynucleotide is operatively linked to a heterologous expression control sequence, such as a promoter. Typically, “heterologous” means derived from a genotypically distinct entity from that of the rest of the entity to which it is being compared. A heterologous nucleic acid sequence refers to any nucleic acid sequence that is not isolated from, derived from, or based upon a naturally occurring nucleic acid sequence of the adenoviral vector.

Examples of constitutive promoters include, without limitation, the TBG promoter, the retroviral Rous sarcoma virus LTR promoter (optionally with the enhancer), the cytomegalovirus (CMV) promoter (optionally with the CMV enhancer, see, e.g., Boshart et al, Cell, 41:521-530 (1985)), the CASI promoter (WO2012/115980), the SV40 promoter, the dihydrofolate reductase promoter, the β-actin promoter, the phosphoglycerol kinase (PGK) promoter, and the EF1a promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. For example, inducible promoters include the zinc-inducible sheep metallothionine (MT) promoter and the dexamethasone (Dex)-inducible mouse mammary tumor virus (MMTV) promoter. Other inducible systems include the T7 polymerase promoter system; the ecdysone insect promoter, the tetracycline-repressible system and the tetracycline-inducible system. Other systems include the FK506 dimer, VP16 or p65 using castradiol, diphenol murislerone, the RU486-inducible system and the rapamycin-inducible system. The effectiveness of some inducible promoters increases over time. In such cases one can enhance the effectiveness of such systems by inserting multiple repressors in tandem, e.g., TetR linked to a TetR by an IRES.

In another embodiment, a native EBV promoter may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

The transgene may be operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally occurring promoters. Examples of promoters that are tissue-specific are known for liver; hepatitis B virus core; alpha-fetoprotein, bone osteocalcin; bone sialoprotein, lymphocytes, immunoglobulin heavy chain; T cell receptor chain), neuronal such as neuron-specific enolase (NSE) promoter, neurofilament light-chain gene, and the neuron-specific vgf gene, among others.

### Vectors

Vectors containing polynucleotides encoding EBV antigen constructs as described herein are also provided. Such vectors will be suitable for delivery to and expression in a host cell. Vectors can be in the form of a replicating or replication defective vector, such as a viral vector. Numerous viral vectors suitable for introducing immunogenic nucleic acids into a subject are known in the art, and include both DNA and RNA viruses. Examples of vectors suitable for encoding EBV antigens described herein include: adenovirus vectors (replicating or replication deficient), pox virus vectors, including vaccinia virus vectors, such as modified vaccinia Ankara virus (MVA), NYVAC, avipox vectors, canarypox (ALVAC) and fowl pox virus (FPV), Alphavirus vectors (such as Sindbis virus, Semliki Forest virus (SFV), Ross River virus, and Venezuelan equine encephalitis (VEE) virus) and chimeras and replicons thereof, herpes virus vectors (e.g., cytomegalovirus (CMV)-derived vectors), arena virus vectors, such as lymphocytic choriomeningitis virus (LCMV) vectors, measles virus vectors, vesicular stomatitis virus vectors, pseudorabies virus, adeno-associated virus, retrovirus, lentivirus, viral like particles, and many others.

In one embodiment, the vector is an adenovirus. The production and use of adenovirus vectors are well known to those of ordinary skill in the art. In the context of the immunogenic combinations disclosed here, examples of disclosure of the design, production and use of adenovirus vectors expressing vaccine antigens can be found in, e.g., in US published application No. 2014/0141042 (WO 2012/089833); U.S. Pat. No. 8,216,834 (WO 2005/071093); US published application No. US 2012/0027788 (WO 2010/086189); and US published application No. US 2005/0214323.

Typically, an adenoviral vector is designed such that the expression cassette is located in a nucleic acid molecule which contains other adenoviral sequences in the region native to a selected adenoviral gene. The expression cassette may be inserted into an existing gene region to disrupt the function of that region, if desired.

Alternatively, the expression cassette may be inserted into the site of a partially or fully deleted adenoviral gene. For example, the expression cassette may be located in the site of a mutation, insertion or deletion which renders non-functional at least one gene of a genomic region selected from the group consisting of E1A, E1B, E2A, E2B, E3 and E4. The term “renders non-functional” means that a sufficient amount of the gene region is removed or otherwise disrupted, so that the gene region is no longer capable of producing functional products of gene expression. If desired, the entire gene region may be removed (and suitably replaced with the expression cassette). Suitably, E1 genes of adenovirus are deleted and replaced with an expression cassette consisting of a promoter of choice, a cDNA sequence of the gene of interest and a poly A signal, resulting in a replication defective recombinant virus.

Adenoviral vectors of use in the present invention may be derived from a range of mammalian hosts. Over 100 distinct serotypes of adenovirus have been isolated which infect various mammalian species, 51 of which are of human origin. Thus one or more of the adenoviral vectors may be derived from a human adenovirus. Examples of such human-derived adenoviruses are Ad1, Ad2, Ad4, Ad5, Ad6, Ad11, Ad24, Ad26, Ad34, Ad35, Ad48, particularly Ad5, Ad11 and Ad35. The human and nonhuman adenoviral serotypes have been categorized into six subgenera (A-F) based on a number of biological, chemical, immunological and structural criteria.

Although Ad5-based vectors have been used extensively in a number of gene therapy trials, there may be limitations on the use of Ad5 and other human group C adenoviral vectors due to preexisting immunity in the general population due to natural infection. Ad5 and other human group C members tend to be among the most seroprevalent serotypes. Immunity to existing vectors may develop as a result of exposure to the vector during treatment. These types of preexisting or developed immunity to seroprevalent vectors may limit the effectiveness of gene therapy or vaccination efforts. Alternative adenovirus serotypes, thus constitute very important targets in the pursuit of gene delivery systems capable of evading the host immune response.

One such area of alternative serotypes are those derived from non-human primates, especially adenoviruses isolated from chimpanzee, bonobos and gorillas. See U.S. Pat. No. 6,083,716 which describes the genome of two chimpanzee adenoviruses.

It has been shown that non-human simian adenoviral vectors induce strong immune responses to transgene products as efficiently as human adenoviral vectors (Fitzgerald et al. (2003) J. Immunol. 170:1416; Colloca et al. (2012) Science Translational Medicine 4:1-9; Roy et al. (2004) Virology 324: 361-372; Roy et al. (2010) J. of Gene Medicine 13:17-25).

Non-human simian adenoviruses can be isolated from the mesenteric lymph nodes or feces of the animals and can replicate in vitro in HEK 293 cells. Despite these similarities, nonhuman simian adenoviruses are phylogenetically and immunologically distinct from the more common human serotypes (Ad2 and Ad5).

Thus, in one embodiment one or more of the adenoviral vectors may be derived from a non-human primate adenovirus eg a chimpanzee adenovirus such as one selected from serotypes ChAd3, ChAd63, ChAd83, ChAd155, Pan5, Pan6, Pan 7 (also referred to as C7) and Pan9. Specifically, the virus may be a non-human adenovirus, such as a simian adenovirus and in particular a chimpanzee adenovirus such as ChAd155, Pan 5, 6, 7 or 9. Examples of such strains are described in US 20040241181 (WO03/000283) and are available from the American Type Culture Collection, 10801 University Boulevard, Manassas, Va. 20110-2209, and other sources. Desirable chimpanzee adenovirus strains include Pan 5 [ATCC VR-591], Pan 6 [ATCC VR-592], and Pan 7 [ATCC VR-593]. Alternatively, adenoviral vectors may be derived from nonhuman simian adenoviruses derived from bonobos, such as PanAd1, PanAd2 or PanAd3. Examples of such vectors described herein can be found for example in US 20110217332 (WO2005/071093), US 2012/0027788 (WO2010/086189) and WO2016/198621.

Use of nonhuman simian adenoviruses is thought to be advantageous over use of human adenovirus serotypes because of low and infrequent pre-existing immunity, in particular the lack of cross-neutralising antibodies, to adenoviruses in the target population. Cross-reaction of the chimpanzee adenoviruses with pre-existing neutralizing antibody responses is only present in 2% of the target population compared with 35% in the case of certain candidate human adenovirus vectors. Pan 6 is less closely related to Pan 5, 7 and 9.

The adenovirus of the invention may be replication defective. This means that it has a reduced ability to replicate in non-complementing cells, compared to the wild type virus. This may be brought about by mutating the virus e.g. by deleting a gene involved in replication, for example deletion of the E1a, E1b, E3 or E4 gene.

The adenoviral vectors in accordance with the present invention may be derived from replication defective adenovirus comprising a functional E1 deletion. Thus the adenoviral vectors according to the invention may be replication defective due to the absence of the ability to express adenoviral E1a and E1b, i.e., are functionally deleted in E1a and E1b. The recombinant adenoviruses may also bear functional deletions in other genes [see, e.g., US 20040241181 (WO 03/000283)] for example, deletions in E3 or E4 genes. The adenovirus delayed early gene E3 may be eliminated from the adenovirus sequence which forms part of the recombinant virus. The function of E3 is not necessary to the production of the recombinant adenovirus particle. Thus, it is unnecessary to replace the function of this gene product in order to package a recombinant adenovirus useful in the invention. In one particular embodiment the recombinant adenoviruses have functionally deleted E1 and E3 genes. The construction of such vectors is described in Roy et al., (2004) Human Gene Therapy 15:519-530.

Recombinant adenoviruses may also be constructed having a functional deletion of the E4 gene, although it may be desirable to retain the E4 ORF6 function. Adenovirus vectors according to the invention may also contain a deletion in the delayed early gene E2a. Deletions may also be made in any of the late genes L1 through to L5 of the adenovirus genome. Similarly deletions in the intermediate genes IX and IVa may be useful.

Other deletions may be made in the other structural or non-structural adenovirus genes. The above deletions may be used individually, i.e. an adenovirus sequence for use in the present invention may contain deletions of E1 only. Alternatively, deletions of entire genes or portions thereof effective to destroy their biological activity may be used in any combination. For example in one exemplary vector, the adenovirus sequences may have deletions of the E1 genes and the E4 gene, or of the E1, E2a and E3 genes, or of the E1 and E3 genes (such as functional deletions in E1 a and E1 b, and a deletion of at least part of E3), or of the E1, E2a and E4 genes, with or without deletion of E3 and so on. Such deletions may be partial or full deletions of these genes and may be used in combination with other mutations, such as temperature sensitive mutations to achieve a desired result. Adenoviral vectors of use in the present invention include PanAd3 (WO 2010/086189) and ChAd155 (WO 2016/198621).

In another embodiment, the viral vector is a pox virus vector. In a specific embodiment, the poxvirus vector is a vaccinia virus vector, such as a modified vaccinia Ankara virus (MVA) vector. (MVA) vector is replication-deficient in humans and other mammals. It was initially developed to improve the safety of smallpox vaccination by passage of Vaccinia virus over 570 times in chicken embryo fibroblast (CEF) cells, resulting in multiple, fully characterised deletions after which the virus was highly attenuated and replication-deficient in humans and other mammals. The replication defect occurs at a late stage of virion assembly such that viral and recombinant gene expression is unimpaired, making MVA an efficient single round expression vector incapable of causing infection in mammals.

MVA has subsequently been extensively used as a viral vector to induce antigen-specific immunity against transgenes, both in animal models and in humans. A description of MVA can be found in Mayr A, et al. “The smallpox vaccination strain MVA: marker, genetic structure, experience gained with the parenteral vaccination and behavior in organisms with a debilitated defense mechanism. “Abstammung, Eigenschaften and Verwendung des attenuierten Vaccinia-Stammes MVA.” Zentralbl Bakteriol B. 1978 December; 167(5-6):375-90 and in Mayr, A., Hochstein-Mintzel, V. & Stick!, H. (1975). Infection 3, 6-14.

In one embodiment, MVA is derived from the virus seed batch 460 MG obtained from the 571st passage of Vaccinia Virus on CEF cells. In a further embodiment, MVA is derived or produced prior to 31 December 1978 and is free of prion contamination.

MVA vectors and methods of production of such vectors are described, for example in U.S. Pat. No. 6,761,893 (WO02/042480); U.S. Pat. Nos. 7,964,395; 7,964,396; US published application no. US 2013/0183335 (WO2012/048817); and US Published Application No. 2015/0209421 (WO2014/019718). Each of the preceding is incorporated herein by reference for the teaching of suitable MVA vectors and methods.

In another embodiment, the viral vector is an Alphavirus vector, such as an alphavirus replicon or other self-replicating RNA vector. Exemplary alphavirus vectors and methods for producing and delivering them suitable for use in the context of the immunogenic combinations disclosed herein are described in, e.g., US20090104226 (WO2006078294); US20110300205 (WO2011005799); US20130195968 (WO 2012/006376); US20130177639 (WO2012006377); WO2013006838; and WO2013006842, each of which are incorporated herein for their disclosure of exemplary self-replicating RNA vectors suitable in the context of the disclosed immunogenic combinations.

Also provided is a method of producing a recombinant viral particle expressing an EBV antigen of the invention, comprising expressing a vector described herein in a host cell. Viral particles can be produced in any suitable cell line in which the viral vector is capable of replication.

Adenoviral vectors can be produced in any suitable cell line in which the virus is capable of replication. In particular, complementing cell lines which provide the factors missing from the viral vector that result in its impaired replication characteristics (such as E1 and/or E4) can be used. Without limitation, such a cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources, such as PER.C6 cells, as represented by the cells deposited under ECACC no. 96022940 at the European Collection of Animal Cell Cultures (ECACC) at the Centre for Applied Microbiology and Research (CAMR, UK) or Her 96 cells (Crucell).

A particularly suitable complementation cell line is the Procell92 cell line. The Procell92 cell line is based on HEK 293 cells which express adenoviral E1 genes, transfected with the Tet repressor under control of the human phosphoglycerate kinase-1 (PGK) promoter, and the G418-resistance gene (Vitelli et al. PLOS One (2013) 8(e55435):1-9). Procell92.S is adapted for growth in suspension conditions and is also useful for producing adenoviral vectors expressing toxic proteins (www.okairos.com/e/inners.php?m=00084, last accessed 13 Apr. 2015).

Vaccinia vectors can be produced according to methods described in the art. For example, preparation and use of MVA vectors is described in Ourmanov et al., J. Virol. (2009) 83:5388-5400; and Martinon et al. Vaccine (2008) 26:532-545.

### Compositions

EBV antigen polypeptides, polynucleotides and vectors described herein may be administered in immunogenic compositions. An immunogenic composition as described herein is a composition comprising one or more recombinant polypeptides, polynucleotides and/or vectors capable of inducing an immune response, for example a humoral (e.g., antibody) and/or cell-mediated (e.g., a cytotoxic T cell) response. following delivery to a mammal, suitably a human.

The immunogenic compositions disclosed herein typically contain one or more pharmaceutically acceptable carriers and/or excipients. Pharmaceutically acceptable carriers and excipients are well known and can be selected by those of skill in the art. The adjective “pharmaceutically acceptable” indicates that the referent is suitable for administration to a subject (e.g., a human or animal subject). Remington's Pharmaceutical Sciences, by E. W. Martin, Mack Publishing Co., Easton, Pa., 15th Edition (1975), describes compositions and formulations (including diluents) suitable for pharmaceutical delivery of therapeutic and/or prophylactic compositions, including immunogenic compositions.

For example, the carrier or excipient can favorably include a buffer. Optionally, the carrier or excipient also contains at least one component that stabilizes solubility and/or stability. Examples of solubilizing/stabilizing agents include detergents, for example, laurel sarcosine and/or tween. Alternative solubilizing/stabilizing agents include arginine, and glass forming polyols (such as sucrose, trehalose and the like). Numerous pharmaceutically acceptable carriers and/or pharmaceutically acceptable excipients are known in the art and are described, e.g., in Remington's Pharmaceutical Sciences, by E. W. Martin, Mack Publishing Co., Easton, Pa., 5th Edition (1975).

Accordingly, suitable excipients and carriers can be selected by those of skill in the art to produce a formulation suitable for delivery to a subject by a selected route of administration.

Suitable excipients include, without limitation: glycerol, Polyethylene glycol (PEG), Sorbitol, Trehalose, N-lauroylsarcosine sodium salt, L-proline, Non detergent sulfobetaine, Guanidine hydrochloride, Urea, Trimethylamine oxide, KCl, Ca2+, Mg2+, Mn2+, Zn2+and other divalent cation related salts, Dithiothreitol, Dithioerytrol, and 13-mercaptoethanol. Other excipients can be detergents (including: Tween80, Tween20, Triton X-00, NP-40, Empigen BB, Octylglucoside, Lauroyl maltoside, Zwittergent 3-08, Zwittergent 3-0, Zwittergent 3-2, Zwittergent 3-4, Zwittergent 3-6, CHAPS, Sodium deoxycholate, Sodium dodecyl sulphate, Cetyltrimethylammonium bromide).

Optionally, an immunogenic composition of the invention may be formulated to contain other components, including, e.g., adjuvants, stabilizers, pH adjusters, preservatives and the like. Examples of suitable adjuvants are provided below under “Adjuvants.”

### Methods of Use

EBV antigen polypeptides, polynucleotides, vectors described herein may be used in the prevention and/or treatment of EBV infection and EBV-associated diseases, e.g., as a vaccine for induction of an immune response. As used herein, induction of an immune response refers to the ability of a protein to induce a T cell and/or a humoral immune response to the protein.

As used herein, induction of an immune response refers to the ability of a protein, also known as an “antigen” or “immunogen,” to induce a T cell and/or a humoral immune response to the protein. For example, an immunogenic composition may induce a memory T and/or B cell population relative to an untreated subject following immunization with the composition, particularly in those embodiments where the composition comprises a nucleic acid comprising a sequence which encodes an EBV antigen polypeptide. In some embodiments, the subject is a vertebrate, such as a mammal e.g. a human or a veterinary mammal.

Immune responses can be measured by methods known in the art, including assays of the induction of proliferation or effector function of the particular lymphocyte type of interest, e.g., B cells, T cells, T cell lines, and T cell clones.

Thus in one embodiment is provided a method of inducing an immune response in a subject comprising administering a polynucleotide, a polypeptide, a vector or an immunogenic composition of the invention to the subject. In one embodiment, the subject is Epstein-Barr virus seronegative. A subject is “seronegative” if the subject has no serological evidence of past or current EBV infection. In another embodiment the subject is Epstein-Barr virus seropositive. A subject is “seropositive” if the subject has serological evidence of past or current EBV infection.

Also provided is a method of treating or preventing an EBV-associated disease in a subject, comprising administering a polynucleotide, a polypeptide, a vector or an immunogenic composition of the invention to the subject. In one embodiment, the EBV-associated disease is an EBV-associated malignancy or an EBV-associated autoimmune disease. EBV-associated diseases, include, for example, multiple sclerosis, rheumatoid arthritis and systemic lupus erythematosus.

Also provided are dosing regimens designed to maximize the immunogenicity of polynucleotides, polypeptides, vectors and immunogenic compositions of the invention. Thus, in one embodiment is provided a method of inducing an immune response in a subject comprising administering two or more doses of a polynucleotide, a polypeptide, a vector and/or an immunogenic composition of the invention to the subject. In certain embodiments the doses are separated by one, two, three, four, five, six, seven, eight, nine, ten, eleven, twelve or more weeks. In another embodiment the doses are separated by one, two, three, four, five, six, seven, eight, nine, ten, eleven, twelve or more months. Alternatively, doses may be separated by one, two, three, four, five, six, seven, eight, nine, ten, eleven, twelve or more years.

In one embodiment is provided a method of inducing an immune response in a subject comprising:
(a) administering an adenovirus vector comprising a polynucleotide of the invention; and
(b) administering a vaccinia virus vector comprising a polynucleotide of the invention;
   wherein steps (a) and (b) are conducted in either order. In one embodiment the adenovirus vector is ChAd155. In another embodiment the vaccinia virus vector is MVA. In one embodiment, step (b) is carried out one, two, three, four, five, six, seven, eight, nine, ten, eleven, twelve or more weeks after step (a). In one embodiment, step (b) is carried out one, two, three, four, five, six, seven, eight, nine, ten, eleven, twelve or more months after step (a).

In one embodiment is provided a method of treating or preventing an EBV-associated disease in a subject comprising:
(a) administering an adenovirus vector comprising a polynucleotide of the invention; and
(b) administering a vaccinia virus vector comprising a polynucleotide of the invention;
   wherein steps (a) and (b) are conducted in either order. In one embodiment the adenovirus vector is ChAd155. In another embodiment the vaccinia virus vector is MVA. In one embodiment, step (b) is carried out one, two, three, four, five, six, seven, eight, nine, ten, eleven, twelve or more weeks after step (a). In one embodiment, step (b) is carried out one, two, three, four, five, six, seven, eight, nine, ten, eleven, twelve or more months after step (a).

Also provided is the use of a polynucleotide, vector, polypeptide, or immunogenic composition of the invention in the manufacture of a medicament for the treatment or prophylaxis of a disease caused by Epstein-Barr Virus infection.

### Adjuvants

An “adjuvant” as used herein refers to a composition that enhances the immune response to an immunogen. A composition according to the invention that comprises an adjuvant can be used as a vaccine, e.g. for human subjects. The adjuvant accelerates, prolongs and/or enhances the quality and/or strength of an immune response to an antigen/immunogen in comparison to the administration of the antigen alone, thus, reduces the quantity of antigen/immunogen necessary in any given vaccine, and/or the frequency of injection necessary in order to generate an adequate immune response to the antigen/immunogen of interest.

Examples of adjuvants that may be used in the context of the compositions of the invention include inorganic adjuvants (e.g. inorganic metal salts such as aluminum phosphate or aluminum hydroxide), gel-like precipitates of aluminum hydroxide (alum); AlPO₄⁻, alhydrogel; bacterial products from the outer membrane of Gram-negative bacteria, in particular monophosphoryl lipid A (MPLA), lipopolysaccharides (LPS), muramyl dipeptides and derivatives thereof; Freund's incomplete adjuvant; liposomes, in particular neutral liposomes, liposomes containing the composition and optionally cytokines; AS01B, AS01E, AS02, non-ionic block copolymers; ISCOMATRIX adjuvant; unmethylated DNA comprising CpG dinucleotides (CpG motif), in particular CpG ODN with a phosphorothioate (PTO) backbone (CpG PTO ODN) or phosphodiester (PO) backbone (CpG PO ODN); synthetic lipopeptide derivatives, in particular Pam₃Cys; lipoarabinomannan; peptidoglycan; zymosan; heat shock proteins (HSP), in particular HSP 70; dsRNA and synthetic derivatives thereof, in particular Poly I:poly C; polycationic peptides, in particular poly-L-arginine; taxol; fibronectin; flagellin; imidazoquinoline; cytokines with adjuvant activity, in particular GM-CSF, interleukin-(IL-2, IL-6, IL-7, IL-18, type I and II interferons, in particular interferon-gamma, TNF-alpha; 25-dihydroxyvitamin D3 (calcitriol); and synthetic oligopeptides, in particular MHCII-presented peptides. Non-ionic block polymers containing polyoxyethylene (POE) and polyoxypropylene (POP), such as POE-POP-POE block copolymers may be used as an adjuvant.

Additional examples of adjuvants include inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, biodegradable microspheres, virosomes, bacterial adjuvants (e.g. monophosphoryl lipid A, such as 3-de-O-acylated monophosphoryl lipid A (3D-MPL), or muramyl peptides), synthetic adjuvants (e.g. monophosphoryl lipid A (MPL), in particular 3-de-O-acylated monophosphoryl lipid A (3D-MPL and muramyl peptide analogues, or synthetic lipid A, and synthetic polynucleotides adjuvants, e.g., polyarginine or polylysine.

Saponins are also suitable adjuvants, for example, the saponin Quil A, derived from the bark of the South American tree Quillaja Saponaria Molina, and fractions thereof. Purified fractions of Quil A are also known as immunostimulants, such as squalene, QS21, QS17 and QS7, a non-haemolytic fraction of Quil-A. Combinations of QS21 and polysorbate or cyclodextrin are also suitable.

Another example of an adjuvant is an immunostimulatory oligonucleotide containing unmethylated cytosine-guanosine dinucleotide motifs present in DNA (“CpG”). CpG is known as an adjuvant when administered by both systemic and mucosal routes. When formulated into vaccines, it may be administered in free solution together with free antigen or covalently conjugated to an antigen or formulated with a carrier such as aluminium hydroxide.

Activation of specific receptors can stimulate an immune response. Such receptors are known to the skilled artisan and comprise, for example, cytokine receptors, in particular type I cytokine receptors, type II cytokine receptors, TNF receptors; and a vitamin D receptor acting as transcription factor; and the Toll-like receptors 1 (TLR1), TLR-2, TLR 3, TLR4, TLR5, TLR-6, TLR7, and TLR9. Agonists to such receptors have adjuvant activity, i.e., are immunostimulatory. Other suitable adjuvants include alkyl glucosaminide phosphates (AGPs) or pharmaceutically acceptable salts of AGPs. Some AGPs are TLR4 agonists, and some are TLR4 antagonists. An adjuvant of the composition of the present invention may be one or more Toll-like receptor agonists. In a more preferred embodiment, the adjuvant is a Toll-like receptor 4 agonist. In a particular preferred embodiment, the adjuvant is a Toll-like receptor 9 agonist.

Adjuvants such as those described above may be formulated together with carriers, such as liposomes, oil in water emulsions, and/or metallic salts (including aluminum salts such as aluminum hydroxide). For example, 3D-MPL may be formulated with aluminum hydroxide or oil in water emulsions; QS21 may be formulated with cholesterol containing liposomes, oil in water emulsions or alum; CpG may be formulated with alum or with other cationic carriers.

Combinations of adjuvants may be utilized in the present invention, in particular a combination of a monophosphoryl lipid A and a saponin derivative, more particularly the combination of QS21 and 3D-MPL or a composition where the QS21 is quenched in cholesterol-containing liposomes (DQ). Alternatively, a combination of CpG plus a saponin such as QS21 is an adjuvant suitable for use in the present invention, as is a potent adjuvant formulation involving QS21, 3D-MPL and tocopherol in an oil in water emulsion. Saponin adjuvants may be formulated in a liposome and combined with an immunostimulatory oligonucleotide. Thus, suitable adjuvant systems include, for example, a combination of monophosphoryl lipid A, preferably 3D-MPL, together with an aluminium salt. A further exemplary adjuvant comprises QS21 and/or MPL and/or CpG. QS21 may be quenched in cholesterol-containing liposomes.

The fusion of the MHC class II invariant chain (also known as CD74) to an antigen which is comprised by an expression system used for vaccination increases the immune response against said antigen, if it is administered with a viral vector, e.g. an adenovirus. Accordingly, in one embodiment of the invention, the immunogenic transgene may be co-expressed with invariant chain in a recombinant ChAd155 viral vector.

In another embodiment, the invention provides the use of the capsid of ChAd155 (optionally an intact or recombinant viral particle or an empty capsid is used) to induce an immunomodulatory response, or to enhance or adjuvant a cytotoxic T cell response to another active agent by delivering a ChAd155 capsid to a subject. The ChAd155 capsid can be delivered alone or in a combination regimen with an active agent to enhance the immune response thereto. Advantageously, the desired effect can be accomplished without infecting the host with an adenovirus.

### Sequence Identity

Identity with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix can be used in conjunction with the computer program. For example, the percent identity can then be calculated as the total number of identical matches multiplied by 100 and then divided by the sum of the length of the longer sequence within the matched span and the number of gaps introduced into the shorter sequences in order to align the two sequences.

Where the present disclosure refers to a sequence by reference to a UniProt or Genbank accession code, the sequence referred to is the current version as of the filing date of the present application.

The skilled person will recognise that individual substitutions, deletions or additions to a protein which alters, adds or deletes a single amino acid or a small percentage of amino acids is an “immunogenic derivative” where the alteration(s) results in the substitution of an amino acid with a functionally similar amino acid or the substitution/deletion/addition of residues which do not substantially impact the immunogenic function.

Conservative substitution tables providing functionally similar amino acids are well known in the art. In general, such conservative substitutions will fall within one of the amino-acid groupings specified below, though in some circumstances other substitutions may be possible without substantially affecting the immunogenic properties of the antigen. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Suitably such substitutions do not occur in the region of an epitope, and do not therefore have a significant impact on the immunogenic properties of the antigen.

Immunogenic derivatives may also include those wherein additional amino acids are inserted compared to the reference sequence. Suitably such insertions do not occur in the region of an epitope, and do not therefore have a significant impact on the immunogenic properties of the antigen. One example of insertions includes a short stretch of histidine residues (e.g. 2-6 residues) to aid expression and/or purification of the antigen in question.

Immunogenic derivatives include those wherein amino acids have been deleted compared to the reference sequence. Suitably such deletions do not occur in the region of an epitope, and do not therefore have a significant impact on the immunogenic properties of the antigen.

The skilled person will recognise that a particular immunogenic derivative may comprise substitutions, deletions and additions (or any combination thereof).

### General

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

The invention will be further described by reference to the following, non-limiting, examples and figures.

### EXAMPLES

### Example 1: Antigen Design

a. EBV Antigen Design

Polyvalent Epstein-Barr Virus antigen constructs were rationally designed with the following design goals in mind:
1) to broadly target EBV antigens that are expressed in EBV-associated multiple sclerosis (EBV-MS) by including multiple latent stage antigens in the design, and optionally including lytic stage antigens;
   2) to reduce the risk of oncogenesis and immune interference by exclusion of known problematic regions, fragmenting and shuffling EBV protein antigens in the polyvalent antigen construct;
   3) to focus the induced immune response on EBV-specific T-cell activation by including T-cell epitopes in the polyvalent antigen constructs; and
   4) to exclude the presence of unwanted anti-self neo-epitopes in the final antigen sequence.

Two polyvalent EBV antigen constructs meeting these criteria were designed. The first polyvalent antigen construct (EBV-L; FIG. 2A) includes immunogenic fragments of latent stage EBV proteins LMP1, LMP2, EBNA1 and EBNA3A. A second construct (EBV-LLy; FIG. 2B) contains the same latent antigen fragments as EBV-L, and also includes an immunogenic fragment of the EBV lytic protein ZEBRA. The EBV latent proteins selected for inclusion in the antigen constructs have been reported to be expressed by B cells in post-mortem brain tissue of multiple sclerosis patients (Serafini et al., J. Exp. Medicine (2007) 204(12):2899; Serafini et al., J. Neuropathol. Exp. Neurol. (2010) 69(7):677. Inclusion of the lytic antigen ZEBRA, a key regulator of the EBV switch from latency to lytic phase, is aimed at controlling the reactivation of the virus and limiting further amplification and spread of EBV.

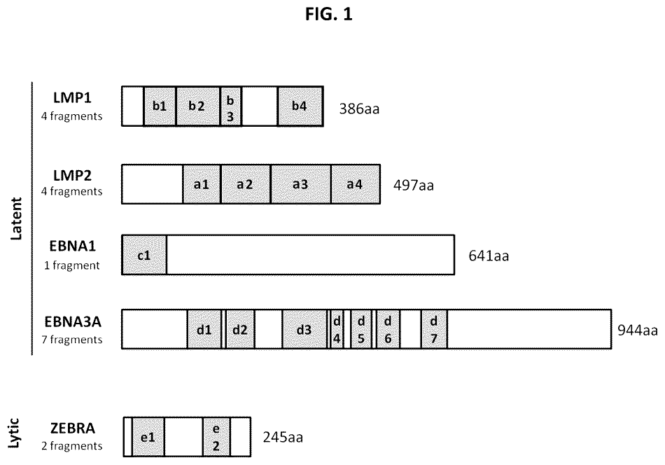

Full length EBV proteins have the potential to transform immune cells. Therefore, to improve the safety of the antigen constructs, problematic regions were excluded and only remaining fragments of the EBV proteins were selected for the construct designs. FIG. 1 illustrates the location of the selected immunogenic fragments in each of the EBV proteins. As illustrated in FIGS. 2A and 2B, the selected fragments were shuffled to produce a polyprotein comprising 16 (EBV-L) or 18 (EBV-LLy) immunogenic fragments, assembled so that fragments derived from the same EBV protein are not adjacent to each other.

Finally, to reduce the risk of unwanted junctional epitopes formed by joining two immunogenic fragments together, a bioinformatics screen was conducted to identify potential anti-self neoepitopes at the junctional areas of candidate antigen constructs. Briefly, sixteen amino acid-long peptides spanning the border (junction) region between each pair of two consecutive antigen fragments were extracted from the vaccine polypeptide sequence (8 amino acids from each antigen fragment). The length of 16 amino acids of the junction region guarantees that each 9mer sequence within the junction region contains amino acids from both antigens. For each 16mer junction peptide, all 9mer peptides were then compared with a collection of 9mer peptides representing the complete human proteome (generated from the NCBI RefSeq peptide database). In no case was a 9mer peptide from a junction region of the vaccine polypeptide sequence found to be present in a human protein.

b. CalHV3 Antigen Design

CalHV3 is a gamma herpesvirus isolated from common marmosets (Callithrix jacchus). Based on similarities in sequence and structure, viral reproduction cycle and pathogenesis, CalHV3 is considered to be the marmoset equivalent of human EBV. See, e.g., Cho et al., PNAS 98(3):1224-1229 (2001). CalHV3 is acquired early in life and is reported to be highly prevalent in natural as well as captive marmoset colonies.

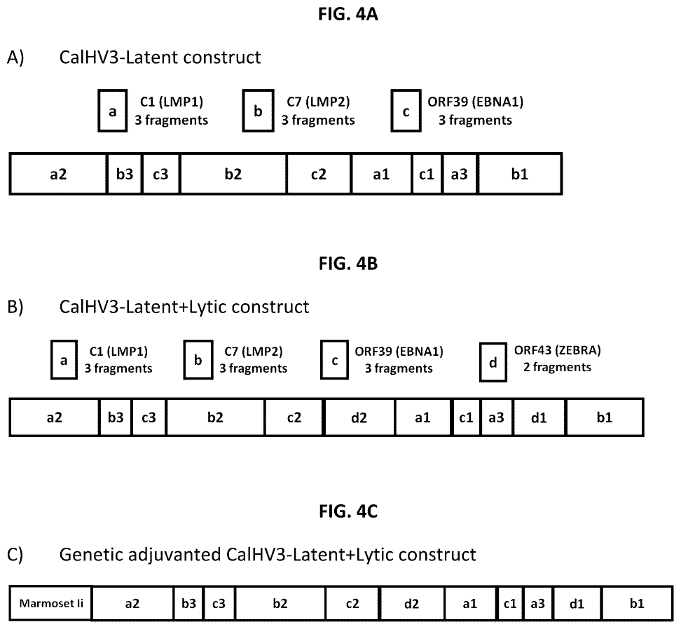

To evaluate the capacity of a similar vaccine to re-expand functional T cell responses to latent and lytic viral antigens in gamma-herpesvirus latently infected individuals in the marmoset model, orthologous CalHV3 antigen constructs weresss developed. Briefly, a CalHV3 latent antigen construct (CalHV3-L, illustrated in FIG. 4A) was constructed from immunogenic fragments of proteins C1 (SEQ ID NO: 28), C7 (SEQ ID NO: 32) and ORF39 (SEQ ID NO: 36), which are the CalHV3 orthologs of EBV LMP1, LMP2 and EBNA1, respectively. As in the EBV-L antigen construct, antigenic regions included in CalHV3-L construct were fragmented and shuffled so that fragments from the same CalHV3 protein are not adjacent to each other. The amino acid sequence of the final CalHV3-L antigen construct is shown in SEQ ID NO: 44 (encoded by the polynucleotide shown in SEQ ID NO: 45).

A CalHV3 Latent/Lytic antigen construct (CalHV3-LLy, illustrated in FIG. 4B) was also constructed. In addition to containing fragments of the latent proteins C1, C7 and ORF39, CalHV3-LLy also contains fragments of ORF43, the CalHV3 ortholog of the EBV ZEBRA protein. The amino acid sequence of the final CalHV3-LLy antigen construct is shown in SEQ ID NO: 46 (encoded by the polynucleotide shown in SEQ ID NO: 47).

Finally, a genetically-adjuvanted version of CalHV3-LLy (Ii-CalHV3-LLy) was constructed by fusing a marmoset MHC class Il-associated invariant chain polypeptide (SEQ ID NO: 43) to the N-terminus of CalHV3-LLy. The amino acid sequence of the final li-CalHV3-LLy antigen construct is shown in SEQ ID NO: 48 (encoded by the polynucleotide shown in SEQ ID NO: 49).

### Example 2: Vector Construction

Polynucleotides encoding EBV-L (SEQ ID NO: 25) and EBV-LLy (SEQ ID NO: 27) were cloned into plasmid pvjTetOhCMV-bghpolyA, containing a tetOhCMV promoter and bovine growth hormone poly-adenylation signal (BGH pA), according to methods described in WO2016/198621. The EBV-L and EBV-LLy expression cassettes were then transferred into a ChAd155 vector backbone by homologous recombination in E. coli BJ5183 competent cells to produce the pChAd155 (ΔE1, ΔE4 Ad5E4 orf6) TetO hCMV-EBV-L (and EBV-LLy) vectors. The nucleic acid sequence of vector pChAd155 (ΔE1, ΔE4_Ad5E4 orf6) TetO hCMV-EBV-L is shown in SEQ ID NO: 50. The antigen-encoding region is at nucleotides 1348-4806 of SEQ ID NO: 50. The nucleic acid sequence of vector pChAd155 (ΔE1, ΔE3, ΔE4_Ad5E4 orf6) TetO hCMV-EBV-LLy expression vector is shown in SEQ ID NO: 51. The antigen-encoding region is at nucleotides 1348-5157 of SEQ ID NO: 51. ChAd155-EBV vector construction was confirmed by transgene sequencing and restriction analysis. The same methods can be used to prepare EBV adenoviral vectors based on alternative modified ChAd155 backbones as described, for example, in WO2016/198621.

The same methods were followed to prepare vectors ChAd155-CalHV3-L, ChAd155-CalHV3-LLy, and ChAd155-li-CalHV3-LLy (encoding CalHV3 antigens CalHV3-L, CalHV3-LLy, and li-CalHV3-LLy, respectively). The nucleic acid sequence of vector pChAd155 (ΔE1, ΔE4_Ad5E4 orf6) TetO hCMV-CalHV3-L is shown in SEQ ID NO: 52 (antigen-encoding region at nucleotides 1348-4482). The nucleic acid sequence of vector pChAd155 (ΔE1, ΔE3, ΔE4_Ad5E4 orf6) TetO hCMV-CalHV3-LLy is shown in SEQ ID NO: 53 (antigen-encoding region at nucleotides 1348-5238). The nucleic acid sequence of vector pChAd155 (AD, ΔE3, ΔE4_Ad5E4 orf6) TetO hCMV-Ii-CalHV3-LLy is shown in SEQ ID NO: 54 (antigen-encoding region at nucleotides 1348-5883).

MVA vectors encoding the EBV-LLy and CalHV3-LLy antigen constructs were also prepared, according to methods known in the art. See, e.g., Ourmanov et al., J. Virol. (2009) 83:5388-5400; and Martinon et al. Vaccine (2008) 26:532-545.

### Example 3: Viral Particle Production

ChAd155 EBV-L and ChAD155 EBV-LLy vectors were linearized with the restriction endonuclease Pmel and transfected into a HEK293-derived cell line (Procell92.S), as described in Vitelli et al., PLOS One (2013) 8(e55435):1-9. These cells are genetically modified to constitutively express the TetO repressor in order to repress transgene expression during virus generation. Viral amplification was performed at small scale (shake flask) and ChAd155-EBV viral particles were purified on double CsCl gradient from 1 liter scale suspension culture. ChAd155-EBV viral particle titers were determined by QPCR targeting the tetOhCMV promoter. The same methods were followed to prepare viral particles from ChAd155-CalHV3-L, ChAd155-CalHV3-LLy, and ChAd155-li-CalHV3-LLy.

Recombinant MVA expressing the EBV-LLy and CalHV3-LLy antigen constructs were obtained using standard methods. Briefly, primary cell cultures of chicken embryo fibroblast (CEF) cells at a defined cell density were infected with MVA-EBV and MVA-CalHV3 viral seed at a defined multiplicity of infection. The MVA-EBV and MVA-CalHV3 virus harvest was purified by fractional gradient centrifugation.

### Example 4: Immunogenicity of ChAd155-EBV and ChAd155-CaIHV3 Antigens in Mice

a. ChAd155-EBV

The immunogenicity of ChAd155-EBV viral particles produced from vectors expressing latent or latent+lytic antigens was evaluated in mice using the experimental design shown in Table 1. Briefly, CB6F1 mice (6 per group) were administered a single dose (10⁶, 10⁷ or 10⁸ viral particles, intramuscularly) of vectors ChAd155-EBV-L or ChAd155-EBV-LLy.

Three weeks after immunization, splenocytes were isolated and assayed for T-cell responses to EBV antigens according to a standard IFNγ ELISpot assay. Briefly, splenocytes from immunized animals were stimulated with overlapping 15mer peptides arranged in 5 pools, each covering the immunogenic fragments from each of the EBV proteins included in the vaccine (LMP1, LMP2, EBNA1, EBNA3A, ZEBRA; n=19 to 84 single peptides/pool). A sixth pool of 16mer peptides (EJ, n=18 peptides) covering each single junction between fragments, and DMSO (the peptide diluent) were also used as stimulants to monitor response to junctional epitopes and as negative control, respectively. T-cell activation was detected by enumerating IFNγ-secreting vaccine-elicited T cells by enzyme-linked immunoSpot (ELISPOT).

TABLE 1

| | | | dose |
|---|---|---|---|
| Group | Vector | n | (vp) |
| 1 | ChAd155 EBV-L | 6 | 10⁸ |
| 2 | ChAd155 EBV-L | 6 | 10⁷ |
| 3 | ChAd155 EBV-L | 6 | 10⁶ |
| 4 | ChAd155 EBV-LLy | 6 | 10⁸ |
| 5 | ChAd155 EBV-LLy | 6 | 10⁷ |
| 6 | ChAd155 EBV-LLy | 6 | 10⁶ |

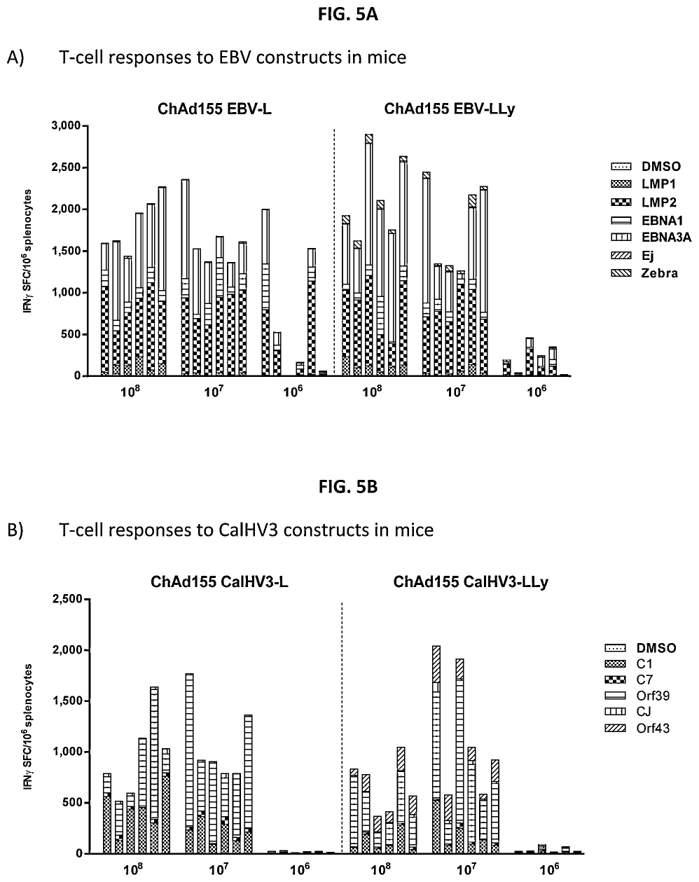

The results are shown in FIG. 5A. Both ChAd155-EBV-L and ChAd155-EBV-LLy elicited T-cells secreting IFNγ in vaccinated mice in a dose-dependent manner. T-cell responses were detected to each of the EBV latent antigens (LMP1, LMP2, EBNA1 and EBNA3A) in both EBV-L and EBV-LLy immunized mice. However, T-cell responses to the EBV lytic protein, ZEBRA, were only detected in EBV-LLy immunized mice. No responses were detected to the EBV junctional peptides (Ej) or to DMSO negative control.

The results indicate that viral particles produced from ChAd155-EBV-L and ChAd155-EBV-LLy vectors are capable of eliciting antigen specific T-cell responses to the immunogenic fragments contained within the antigen constructs. Furthermore, the primary immune response to EBV-L and EBV-LLy antigen constructs is not directed to junctional epitopes.

b. ChAd155-CalHV3

The immunogenicity of ChAd155-CalHV3 viral particles was assessed following the same methods described above for ChAd155-EBV viral particles. Antigen stimulation of splenocytes 3 weeks post vaccination was carried out with overlapping 15mer peptides arranged in 4 pools, each covering the immunogenic fragments included in the vaccine (C1, C7, ORF39, ORF43, n=58 to 96 single peptides/pool). A fifth pool of 16mer peptides (CJ, n=12 peptides) covering each single junction between fragments, and DMSO (the peptide diluent) were also used as stimulants to monitor response to junctional epitopes and as negative control, respectively. The experimental design is summarized in Table 2.

TABLE 2

| | | | dose |
|---|---|---|---|
| Group | Vector | n | (vp) |
| 1 | ChAd155 CalHV3-L | 6 | 10⁸ |
| 2 | ChAd155 CalHV3-L | 6 | 10⁷ |
| 3 | ChAd155 CalHV3-L | 6 | 10⁶ |
| 4 | ChAd155 CalHV3-LLy | 6 | 10⁸ |
| 5 | ChAd155 CalHV3-LLy | 6 | 10⁷ |
| 6 | ChAd155 CalHV3-LLy | 6 | 10⁶ |

The results are shown in FIG. 5B. Both ChAd155-CalHV3-L and ChAd155-CalHV3-LLy elicited T cells secreting IFNγ in vaccinated mice in a dose-dependent manner. T-cell responses to peptide pools covering CalHV3 latent antigens C1, C7 and ORF39 were detected in both CalHV3-L and CalHV3-LLy immunized mice. However, T-cell responses to the CalHV3 lytic protein, ORF43, were only detected in CalHV3-LLy immunized mice. No responses were detected to the CalHV3 junctional peptides (Cj) or to the negative control, DMSO.

The results indicate that viral particles produced from ChAd155-EBV-L and ChAd155-EBV-LLy vectors are capable of eliciting antigen specific T-cell responses to the immunogenic fragments contained within the antigen constructs. Furthermore, the primary immune response to CalHV3-L and CalHV3-LLy antigen constructs is not directed to junctional epitopes.

### Example 5: Effect of Prime-Boost in Mice

a. EBV-LLy Prime-Boost

The ability of a second dose of EBV-LLy antigen to boost the immune response of a first dose of EBV-LLy antigen was evaluated using the experimental design summarized in Table 3. Briefly, groups of CB6F1 mice (n=5/group) were immunized intramuscularly on day 0 with 5×10⁷ viral particles of Chad155-EBV-LLy. On day 21 (week 3), Group 2 received a second immunization with 10⁷ plaque forming units (PFU) of MVA-EBV-LLy. Control mice received either no additional immunizations (Group 3: “no boost”) or a boost immunization with an MVA vector encoding an EBV unrelated antigen (Group 1: MVA-unrelated).

TABLE 3

| Group | Prime (w0) | Boost (w3) | n | dose (vp or pfu) |
|---|---|---|---|---|
| 1 | ChAd155 | MVA-unrelated | 5 | 10⁷ vp/10⁷ pfu |
| | EBV-LLy | | | |
| 2 | ChAd155 | MVA EBV-LLy | 5 | 10⁷ vp/10⁷ pfu |
| | EBV-LLy | | | |
| 3 | ChAd155 | no boost | 5 | 10⁷ vp |
| | EBV-LLy | | | |

Four weeks after the first immunization, splenocytes were isolated from the mice and antigen-specific T-cell responses were assessed using the methods described in Example 4.

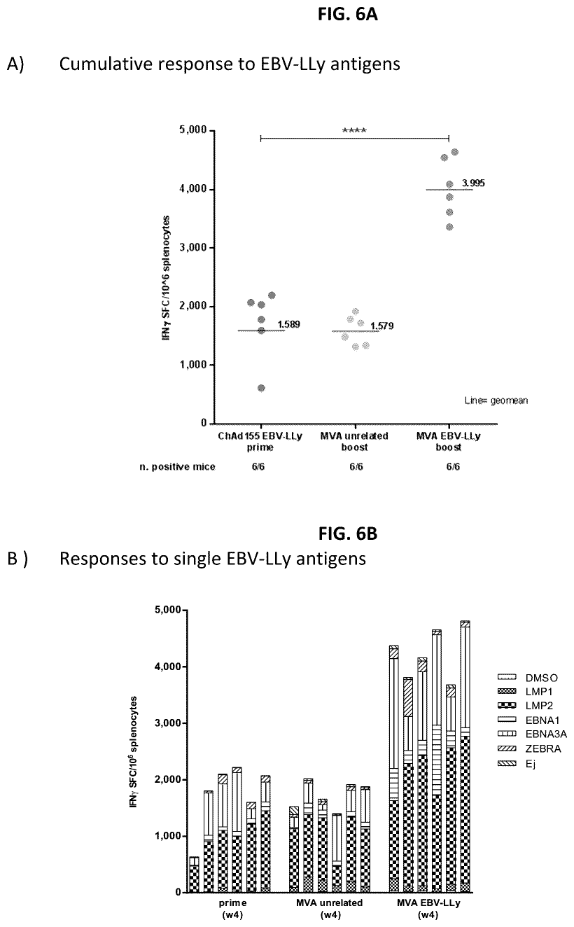

As shown in FIGS. 6A and 6B, immunization with ChAd155-EBV-LLy followed by a boost immunization with MVA-EBV-LLy produced a significant increase in EBV-specific interferon gamma release, as compared to unboosted mice or mice receiving a “boost” injection of an unrelated antigen. FIG. 6A presents the cumulative T-cell responses to all antigens (LMP1, LMP2, EBNA1, EBNA3A and ZEBRA), and FIG. 6B shows the responses to individual antigens.

b. CalHV3-LLy Prime-Boost

The ability to boost immune responses to CalHV3 antigens was assessed using the experimental design summarized in Table 4. Briefly, groups of CB6F1 mice (n=6/group) were immunized intramuscularly on day 0 with 5×10⁷ viral particles of Chad155-CalHV3-LLy. On day 42 (week 6), Group 4 received a second immunization with same ChAd155-CalHV3-LLy antigen construct, while Group 3 received a boost immunization with MVA-CalHV3-LLy. Control mice received either no boost (Group 1) or a boost with an MVA encoding for a CalHV3-unrelated antigen (MVA-unrelated).

TABLE 4

| Group | Prime (w0) | Boost (w6) | n | dose (vp or pfu) |
|---|---|---|---|---|
| 1 | ChAd155 | no boost | 6 | 5 × 10⁷ vp |
| | CalHV3-LLy | | | |
| 2 | ChAd155 | MVA- | 6 | 5 × 10⁷ vp/10⁷ pfu |
| | CalHV3-LLy | unrelated | | |
| 3 | ChAd155 | MVA | 6 | 5 × 10⁷ vp/10⁷ pfu |
| | CalHV3-LLy | CalHV3-LLy | | |
| 4 | ChAd155 | ChAd155 | 6 | 5 × 10⁷ vp/ |
| | CalHV3-LLy | CalHV3-LLy | | 5 × 10⁷ vp |

Seven weeks after the first immunization, splenocytes were isolated from the mice and antigen-specific T-cell responses were assessed using the methods described in Example 4.

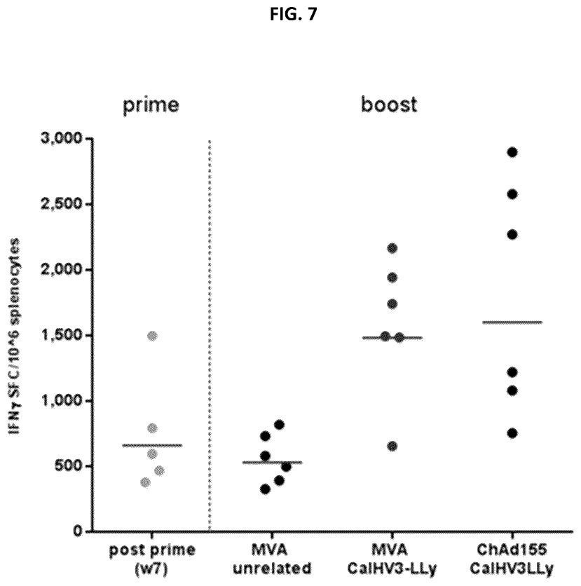

As shown in FIG. 7, immunization with ChAd155-CalHV3-LLy followed by a boost immunization with either the same antigen construct (ChAd155-CalHV3) or MVA-CalHV3-LLy produced a significant increase in CalHV3-specific interferon gamma release at week 7, as compared to unboosted mice or mice receiving a “boost” injection of an unrelated antigen.

These results demonstrate the ability to enhance the immune response to EBV and CalHV3 antigen constructs using prime-boost regimens.

### Example 6: Invariant Chain-CalHV3-LLy Fusion Proteins

Fusing antigens to the major histocompatibility complex (MHC) class II—associated invariant chain (Ii) has been reported to enhance antigen-specific T-cell responses. See, e.g., Capone et al., Mol Ther. 2014 May; 22(5): 1039-1047. Therefore, the immunogenicity of viral particles expressing the marmoset invariant chain (Ii) fused to the N-terminus of the CalHV3-LLy antigen polypeptide was assessed in CB6F1 mice according to the study design outlined in Table 5. Antigen-specific T-cell responses were assessed using the IFNγ assays described in Example 4.

TABLE 5

| Group | Prime (w0) | n | dose (vp) |
|---|---|---|---|
| 1 | ChAd155 CalHV3-LLy | 6 | 5 × 10⁷ vp |
| 2 | ChAd155 CalHV3-LLy | 6 | 5 × 10⁶ vp |
| 3 | ChAd155 marmoset li CalHV3-LLy | 6 | 5 × 10⁷ vp |
| 4 | ChAd155 marmoset li CalHV3-LLy | 6 | 5 × 10⁶ vp |

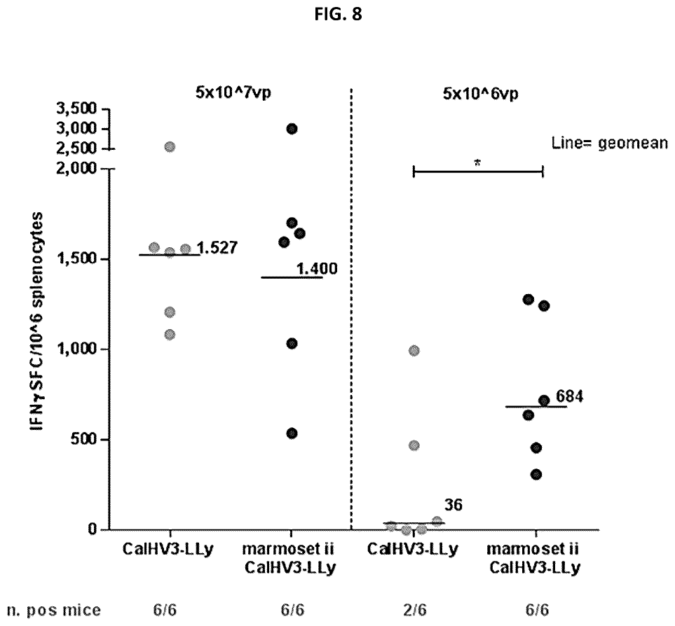

FIG. 8 summarizes cumulative T-cell responses to all CalHV3-LLy antigens (C1, C7, ORF39 and ORF43) observed two weeks after immunization. At the lower dose of antigen (5×10⁶ viral particles), ChAd155-li-CalHV3-LLy elicited significantly greater IFNγ release in immunized mice than ChAd155-CalHV3-LLy. No differences were observed at the higher antigen dose tested (5×10⁷ viral particles).

These results indicate that fusion of the CalHV3-LLy antigen polypeptide to an MHC class II-associated invariant chain polypeptide enhances the T-cell immune response to CalHV3 latent and lytic antigens.

### Example 7: Immunogenicity of CalHV3 Antigens in Non-Human Primates

The immunogenicity of CalHV3 antigen constructs was evaluated in CalHV3 seropositive marmosets (Callithrix jacchus), a genus of New World primates. CalHV3 infection is known to be prevalent in marmosets. See, e.g., Cho et al., PNAS 98(3):1224-1229 (2001). Due to the structural and pathological similarities between CalHV3 and EBV, CalHV3-positive marmosets can serve as a valuable model of human EBV infection and pathology. Id.

Animals were immunized using a ChAd155 prime/MVA boost vaccination schedule. Briefly, a group composed of four adult animals (3 males and one female) received a prime immunization of ChAd155-CalHV3-LLy (5×10¹⁰ vp) on day 0 and a boost immunization of MVA-CalHV3-LLy (2×10⁸) on day 56 (week 8). Blood samples were drawn 2 weeks before prime injections, 3 weeks after prime injections, and 1, 4 and 7 weeks after boost injections. Antigen-specific T-cell responses in peripheral blood mononuclear cells (PBMCs) were assessed using the methods described in Example 4.

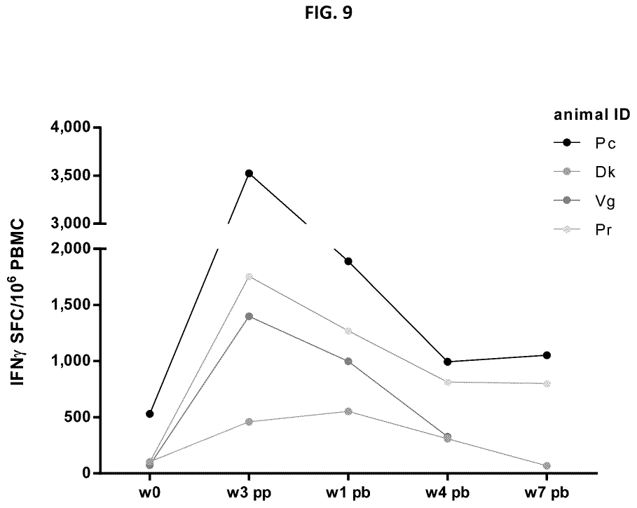

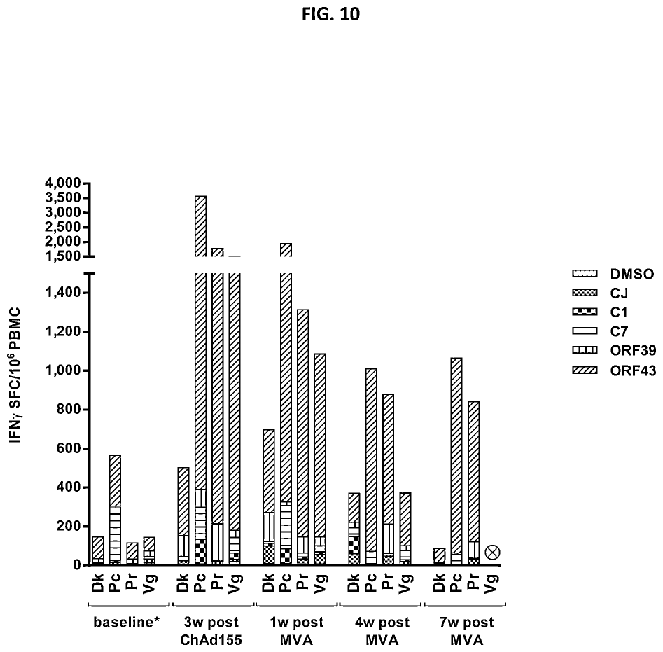

FIG. 9 shows cumulative T-cell responses in individual animals to all CalHV3-LLy antigens (01, C7, ORF39 and ORF43) observed at baseline (w0), at three weeks post-prime (w3 pp); and at one, four and seven weeks post-boost (w1, w4, w7 pb). Prior to immunizations, animals exhibited baseline CalHV3-specific T-cell responses, consistent with the fact that the animals are virus carriers. Three weeks after ChAd155-CalHV3-LLy immunization, animals exhibited significant expansion of pre-existing CalHV3-specific T cell responses. T cell responses continued to be elevated 1 week post-boost with MVA-CalHV3-LLy, and contracted in the following 2 months, still remaining above baseline levels in most animals. As shown in FIG.

10, the enhanced T-cell responses to CalHV3-LLy was sustained and was polyspecific (i.e., to C1, C7, ORF39 and ORF43).

These results indicate that ChAd155 and MVA vectors encoding CalHV3-LLy antigens can efficiently re-expand and sustain pre-existing antigen-specific T-cell responses in CalHV3-positive marmosets, and that circulating T cells elicited by gamma herpesviruses are not functionally impaired or exhausted.

### Example 8: Recognition of EBV Fragments Encoded by ChAd155 and MVA Latent+Lytic EBV Vaccines by Human PBMC

To validate the selection of EBV latent (EBNA1, EBNA3A, LMP1 and LMP2) and lytic (ZEBRA) antigenic fragments included in the vaccine, T cell responses to the corresponding peptide pools were measured in otherwise healthy human EBV carriers.

Briefly, frozen peripheral blood mononuclear cells (PBMC) from 8 healthy human donors were thawed and assayed for T-cell responses to EBV antigens according to a standard IFNγ ELISpot assay. PBMC were plated in triplicates at 2×10⁵ cells/well and were stimulated overnight with overlapping 15-mer peptides arranged in 5 pools, each covering the immunogenic fragments from each of the EBV proteins included in the vaccine (LMP1, LMP2, EBNA1, EBNA3A, ZEBRA; n=19 to 84 single peptides/pool). Stimulation with DMSO (the peptide diluent) was used as negative control. T-cell activation was detected by enumerating IFNγ-secreting T cells by enzyme-linked immunoSpot (ELISPOT).

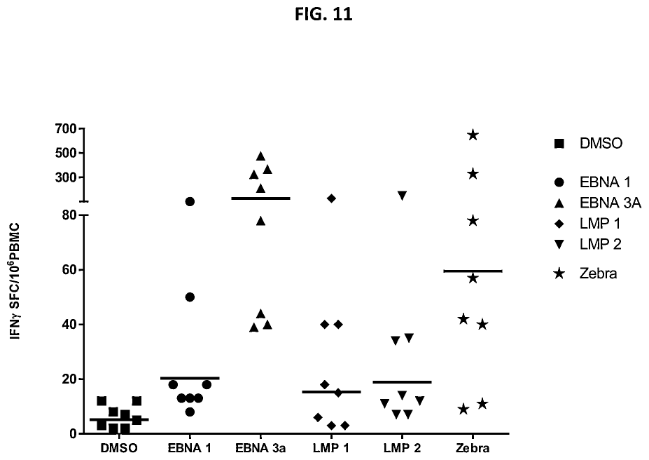

As shown in FIG. 11, the results indicate that antigen specific T-cell responses to the immunogenic fragments contained within the EBV Lly antigen constructs can be readily detected in healthy EBV carriers, with EBNA3A and ZEBRA being the most frequently recognized and eliciting the highest responses, consistent with previous reports (e.g., Taylor et al. Ann. Rev. Immunol. 33:787-821, 2015).

1. A polynucleotide encoding a polypeptide, wherein the polypeptide comprises an immunogenic fragment comprising the following sequence:
(a) one or more of SEQ ID NOs: 2-5 (LMP1 fragment);
(b) one or more of SEQ ID NOs: 7-10 (LMP2 fragment);
(c) one or more sequences of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11 (EBNA1 fragment);
(d) one or more of SEQ ID NOs: 14-20 (EBNA3A fragment); and
(e) one or more of SEQ ID NOs: 22-23 (ZEBRA fragment),
wherein the polynucleotide is operatively linked to one or more sequences which direct expression of said polypeptide in a host cell.

2. (canceled)

3. The polynucleotide of claim 1, wherein the encoded polypeptide comprises:
(a) two or more of SEQ ID NOs: 2-5 (LMP1 fragment);
(b) two or more of SEQ ID NOs: 7-10 (LMP2 fragment);
(c) two or more sequences of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11 (EBNA1 fragment);
(d) two or more of SEQ ID NOs: 14-20 (EBNA3A fragment); or
(e) SEQ ID NOs: 22 and 23 (ZEBRA fragment).

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. The polynucleotide claim 3, wherein the encoded polypeptide comprises:
immunogenic fragments of LMP1 are not adjacent to each other;
immunogenic fragments of LMP2 are not adjacent to each other;
immunogenic fragments of EBNA1 are not adjacent to each other;
immunogenic fragments of EBNA3 are not adjacent to each other; and/or
immunogenic fragments of ZEBRA are not adjacent to each other.

17. The polynucleotide of claim 1, wherein the encoded polypeptide comprises:
(a) a first and a second fragment of LMP1, wherein said first and second fragments of LMP1 are selected from the group consisting SEQ ID NOs: 2-5 and wherein said first and second fragments of LMP1 are not adjacent to each other in the polypeptide;
(b) a first and a second fragment of LMP2, wherein said first and second fragments of LMP2 are selected from the group consisting SEQ ID NOs: 7-10 and wherein said first and second fragments of LMP2 are not adjacent to each other in the polypeptide;
(c) a fragment of EBNA1 consisting of SEQ ID NO: 12; and
(d) a first and a second fragment of EBNA3A, wherein said first and second fragments of EBNA3A are selected from the group consisting SEQ ID NOs: 14-20 and wherein said first and second fragments of EBNA3A are not adjacent to each other in the polypeptide.

18. The polynucleotide of claim 17, wherein the encoded polypeptide comprises:
(a) a first fragment of LMP1 consisting of SEQ ID NO: 2;
(b) a second fragment of LMP1 consisting of SEQ ID NO: 3;
(c) a third fragment of LMP1 consisting of SEQ ID NO: 4;
(d) a fourth fragment of LMP1 consisting of SEQ ID NO: 5;
(e) a first fragment of LMP2 consisting of SEQ ID NO: 7;
(f) a second fragment of LMP2 consisting of SEQ ID NO: 8;
(g) a third fragment of LMP2 consisting of SEQ ID NO: 9;
(h) a fourth fragment of LMP2 consisting of SEQ ID NO: 10;
(i) a first fragment of EBNA1 consisting of SEQ ID NO: 12;
(j) a first fragment of EBNA3A consisting of SEQ ID NO: 14;
(k) a second fragment of EBNA3A consisting of SEQ ID NO: 15;
(l) a third fragment of EBNA3A consisting of SEQ ID NO: 16;
(m) a fourth fragment of EBNA3A consisting of SEQ ID NO: 17;
(n) a fifth fragment of EBNA3A consisting of SEQ ID NO: 18;
(o) a sixth fragment of EBNA3A consisting of SEQ ID NO: 19;
(p) a seventh fragment of EBNA3A consisting of SEQ ID NO: 20;
(q) a first fragment of ZEBRA consisting of SEQ ID NO: 22; and
(r) a second fragment of ZEBRA consisting of SEQ ID NO: 23,
wherein the first, second, third and fourth LMP1 fragments are not adjacent to each other; the first, second, third and fourth LMP2 fragments are not adjacent to each other; and the first, second, third, fourth, fifth, sixth, and seventh EBNA3A fragments are not adjacent to each other; and the first and second ZEBRA fragments are not adjacent to each other.

19. (canceled)

20. The polynucleotide of claim 1, wherein the encoded polypeptide is at least 80%, at least 85%, at least 90%, at least 95% or at least 99% identical to SEQ ID NO: 24 or SEQ ID NO: 26.

21. (canceled)

22. A vector comprising the polynucleotide of claim 20.

23. (canceled)

24. (canceled)

25. (canceled)

26. (canceled)

27. (canceled)

28. (canceled)

29. A polypeptide encoded by the polynucleotide of claim 1 or the vector of claim 22 which is at least 80%, at least 85%, at least 90%, at least 95% or at least 99% identical to SEQ ID NO: 24 or SEQ ID NO: 26.

30. (canceled)

31. An immunogenic composition comprising the polynucleotide of claim 1, the vector of claim 22, or the polypeptide of claim 29; and a pharmaceutically acceptable excipient.

32. The immunogenic composition of claim 31, further comprising an adjuvant.

33. (canceled)

34. (canceled)

35. A method of producing a recombinant viral particle capable of expressing an EBV antigen, comprising expressing the vector of claim 22 in a host cell.

36. A method of inducing an immune response in a subject comprising administering the polynucleotide of claim 1, the vector of claim 22, the polypeptide of claim 29, or the immunogenic composition of claim 31 to the subject.

37. The method of claim 36, wherein the subject is Epstein-Barr virus seronegative.

38. The method of claim 36, wherein the subject is Epstein-Barr virus seropositive.

39. A method of treating or preventing an EBV-associated disease in a subject, comprising administering the polynucleotide of claim 1, the vector of claim 22, the polypeptide of claim 29, or the immunogenic composition of claim 31 to the subject.

40. The method of claim 39, wherein the EBV-associated disease is an EBV-associated autoimmune disease or an EBV-associated malignancy.

41. The method of claim 39, wherein the EBV-associated disease is selected from the group consisting of multiple sclerosis, rheumatoid arthritis and systemic lupus erythematosus.

42. (canceled)

43. (canceled)

44. (canceled)

45. An immunogenic composition comprising one or more polynucleotide(s) encoding one or more polypeptide(s) comprising an immunogenic fragment, wherein the immunogenic fragments encoded by the composition comprise the following sequence:
(a) one or more of SEQ ID NOs: 2-5 (LMP1 fragment);
(b) one or more of SEQ ID NOs: 7-10 (LMP2 fragment);
(c) one or more sequences at least one immunogenic fragment of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11 (EBNA1 fragment);
(d) one or more of SEQ ID NOs: 14-20 (EBNA3A fragment); and
(e) one or more of SEQ ID NOs: 22-23 (ZEBRA fragment);
wherein the polynucleotide is operatively linked to one or more sequences which direct expression of said polypeptide in a host cell.

46. The immunogenic composition of claim 45, wherein the immunogenic fragments encoded by the composition comprise:
(a) two or more of SEQ ID NOs: 2-5 (LMP1 fragment);
(b) two or more of SEQ ID NOs: 7-10 (LMP2 fragment);
(c) two or more sequences of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11 (EBNA1 fragment);
(d) two or more of SEQ ID NOs: 14-20 (EBNA3A fragment); and
(e) SEQ ID NOs: 22 and 23 (ZEBRA fragment).

47. The immunogenic composition of claim 45, wherein the immunogenic fragments encoded by the composition comprise:
(a) SEQ ID NOs: 2-5 (LMP1 fragment);
(b) SEQ ID NOs: 7-10 (LMP2 fragment);
(c) three or more sequences of at least 8, at least 9, at least 10, at least 11, at least 12, at least 13, at least 14, at least 15, at least 20, at least 30, at least 40, at least 50, at least 60, at least 70, at least 80 at least 90 or at least 100 amino acids of SEQ ID NO: 11 (EBNA1 fragment);
(d) SEQ ID NOs: 14-20 (EBNA3A fragment); and
(e) SEQ ID NOs: 22 and 23 (ZEBRA fragment).


SEQUENCE LISTING (54 sequences)

SEQ ID NO: 1 - protein, 386 aa, unidentified
  source 1..386: note = Human herpesvirus 4
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 2 - protein, 62 aa, unidentified
  source 1..62: note = Human herpesvirus 4
MSDWTGGALLVLYSFALMLIIIILIIFIFRRDLLCPLGALCILLLMITLLLIALWNLHGQAL

SEQ ID NO: 3 - protein, 85 aa, unidentified
  source 1..85: note = Human herpesvirus 4
FLGIVLFIFGCLLVLGIWIYLLEMLWRLGATIWQLLAFFLAFFLDLILLIIALYLQQNWWTLLVDLLWLLLFLAILIWMYYHGQR

SEQ ID NO: 4 - protein, 40 aa, unidentified
  source 1..40: note = Human herpesvirus 4
HSDEHHHDDSLPHPQQATDDSGHESDSNSNEGRHHLLVSG

SEQ ID NO: 5 - protein, 114 aa, unidentified
  source 1..114: note = Human herpesvirus 4
NGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 6 - protein, 497 aa, unidentified
  source 1..497: note = Human herpesvirus 4
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 7 - protein, 71 aa, unidentified
  source 1..71: note = Human herpesvirus 4
MNPVCLPVIVAPYLFWLAAIAASCFTASVSTVVTATGLALSLLLLAAVASSYAAAQRKLLTPVTVLTAVVT

SEQ ID NO: 8 - protein, 94 aa, unidentified
  source 1..94: note = Human herpesvirus 4
FFAICLTWRIEDPPFNSLLFALLAAAGGLQGIYVLVMLVLLILAYRRRWRRLTVCGGIMFLACVLVLIVDAVLQLSPLLGAVTVVSMTLLLLAF

SEQ ID NO: 9 - protein, 115 aa, unidentified
  source 1..115: note = Human herpesvirus 4
VLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLI

SEQ ID NO: 10 - protein, 98 aa, unidentified
  source 1..98: note = Human herpesvirus 4
VAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 11 - protein, 641 aa, unidentified
  source 1..641: note = Human herpesvirus 4
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 12 - protein, 86 aa, unidentified
  source 1..86: note = Human herpesvirus 4
MSDEGPGTGPGNGLGEKGDTSGPEGSGGSGPQRRGGDNHGRGRGRGRGRGGGRPGAPGGSGSGPRHRDGVRRPQKRPSCIGCKGTH

SEQ ID NO: 13 - protein, 944 aa, unidentified
  source 1..944: note = Human herpesvirus 4
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 14 - protein, 66 aa, unidentified
  source 1..66: note = Human herpesvirus 4
IRDRRRNPASRRDQAKWRLQTLAAGWPMGYQAYSSWMYSYTDHQTTPTFVHLQATLGCTGGRRCHV

SEQ ID NO: 15 - protein, 55 aa, unidentified
  source 1..55: note = Human herpesvirus 4
TFSAGTFKLPRCTPGDRQWLYVQSSVGNIVQSCNPRYSIFFDYMAIHRSLTKIWE

SEQ ID NO: 16 - protein, 88 aa, unidentified
  source 1..88: note = Human herpesvirus 4
WIDDNPSTETAQAWNAGFLRGRAYGIDLLRTEGEHVEGATGETREESEDTESDGDDEDLPCIVSRGGPKVKRPPIFIRRLHRLLLMRA

SEQ ID NO: 17 - protein, 25 aa, unidentified
  source 1..25: note = Human herpesvirus 4
GKRTEQGKEVLEKARGSTYGTPRPP

SEQ ID NO: 18 - protein, 40 aa, unidentified
  source 1..40: note = Human herpesvirus 4
AQVPEPPTIHLAAQGMAYPLHEQHGMAPCPVAQAPPTPLP

SEQ ID NO: 19 - protein, 62 aa, unidentified
  source 1..62: note = Human herpesvirus 4
GRVACAPVPAPAGPIVRPWEPSLTQAAGQAFAPVRPQHMPVEPVPVPTVALERPVYPKPVRP

SEQ ID NO: 20 - protein, 51 aa, unidentified
  source 1..51: note = Human herpesvirus 4
RPAPWTPNPPRSPSQMSVRDRLARLRAEAQVKQASVEVQPPQLTQVSPQQP

SEQ ID NO: 21 - protein, 245 aa, unidentified
  source 1..245: note = Human herpesvirus 4
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 22 - protein, 66 aa, unidentified
  source 1..66: note = Human herpesvirus 4
DLGGPSQAPLPCVLWPVLPEPLPQGQLTAYHVSTAPTGSWFSAPQPAPENAYQAYAAPQLFPVSDI

SEQ ID NO: 23 - protein, 51 aa, unidentified
  source 1..51: note = Human herpesvirus 4
RKPQQPESLEECDSELEIKRYKNRVASRKCRAKFKQLLQHYREVAAAKSSE

SEQ ID NO: 24 - protein, 1153 aa, synthetic construct
  REGION 1..1153: note = Description of Artificial Sequence: Synthetic polypeptide
MRPAPWTPNPPRSPSQMSVRDRLARLRAEAQVKQASVEVQPPQLTQVSPQQPVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTVIRDRRRNPASRRDQAKWRLQTLAAGWPMGYQAYSSWMYSYTDHQTTPTFVHLQATLGCTGGRRCHVFLGIVLFIFGCLLVLGIWIYLLEMLWRLGATIWQLLAFFLAFFLDLILLIIALYLQQNWWTLLVDLLWLLLFLAILIWMYYHGQRGRVACAPVPAPAGPIVRPWEPSLTQAAGQAFAPVRPQHMPVEPVPVPTVALERPVYPKPVRPVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIHSDEHHHDDSLPHPQQATDDSGHESDSNSNEGRHHLLVSGAQVPEPPTIHLAAQGMAYPLHEQHGMAPCPVAQAPPTPLPFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYDGKRTEQGKEVLEKARGSTYGTPRPPMSDWTGGALLVLYSFALMLIIIILIIFIFRRDLLCPLGALCILLLMITLLLIALWNLHGQALMSDEGPGTGPGNGLGEKGDTSGPEGSGGSGPQRRGGDNHGRGRGRGRGRGGGRPGAPGGSGSGPRHRDGVRRPQKRPSCIGCKGTHWIDDNPSTETAQAWNAGFLRGRAYGIDLLRTEGEHVEGATGETREESEDTESDGDDEDLPCIVSRGGPKVKRPPIFIRRLHRLLLMRAMNPVCLPVIVAPYLFWLAAIAASCFTASVSTVVTATGLALSLLLLAAVASSYAAAQRKLLTPVTVLTAVVTTFSAGTFKLPRCTPGDRQWLYVQSSVGNIVQSCNPRYSIFFDYMAIHRSLTKIWE

SEQ ID NO: 25 - DNA, 3459 bp, synthetic construct
  misc_feature 1..3459: note = Description of Artificial Sequence: Synthetic polynucleotide
  source 1..3459: mol_type = other DNA
atgagacctgctccctggacacctaatcctcccaggtcccccagccagatgagcgtgagagacagactggctaggctgagagccgaggctcaggtcaagcaggccagcgtcgaggtgcaaccccctcagctcacccaggtgtccccccagcagcctgtggccggcattctgttcattctggccattctgaccgagtggggaagcggcaacagaacctacggccctgtcttcatgtgcctcggaggactgctgacaatggtggctggcgccgtgtggctcaccgtcatgtccaacaccctgctcagcgcctggattctgaccgccggattcctgatctttctgatcggattcgctctctttggcgtcatcaggtgttgcaggtactgttgctactactgcctgaccctcgagagcgaggaaagaccccccaccccctacaggaatacagtgattagggacagaaggaggaatcctgcctccaggagagaccaggccaaatggagactccaaacactcgccgctggatggcccatgggctaccaggcctatagctcctggatgtacagctacaccgaccatcagacaacacccaccttcgtgcatctgcaggctacactgggctgcaccggaggcagaaggtgtcacgtgtttctgggaatcgtgctgttcatctttggatgcctgctcgtgctgggcatctggatttatctcctggagatgctctggagactcggcgctacaatttggcagctgctcgccttttttctggccttctttctggacctgatcctcctgatcatcgccctgtacctccaacagaactggtggaccctcctggtggatctgctgtggctcctcctcttcctggccatcctgatctggatgtactaccatggccagagaggaagggtcgcttgcgctcctgtccctgctcctgctggccccatcgtgaggccttgggagccttccctcacacaggccgccggccaggcctttgctcccgtgaggccccagcacatgcctgtggaacccgtgcccgtccccacagtggctctggaaaggcctgtgtaccccaagcccgtgagacctgtcctctggctcagcagccctggaggactcggaacactcggagccgctctcctgacactggccgctgctctggctctgctggctagcctgatcctgggaaccctcaacctcaccaccatgtttctcctcatgctcctgtggaccctcgtggtgctgctcatctgttccagctgctccagctgccccctgagcaagatcctgctggccaggctgttcctgtacgccctcgccctcctgctgctggctagcgccctgatcgctggcggaagcatcctccagaccaatttcaagagcctctcctccaccgagttcatccccaacctgttctgtatgttactgctgatccatagcgacgagcaccatcatgacgactccctgccccatcctcagcaggccacagacgactccggccacgagagcgacagcaatagcaatgagggcaggcaccatctgctcgtgtccggagctcaagtccccgagcctcccaccatccatctcgccgcccagggaatggcttaccccctccacgagcagcacggcatggccccttgtcccgtcgctcaagccccccctacacctctgccctttttcgccatttgtctgacctggagaatcgaggacccccccttcaacagcctgctgttcgccctgctcgccgccgctggcggcctccagggcatttacgtcctcgtgatgctggtgctgctgatcctcgcttacaggagaagatggaggagactgacagtgtgcggcggcatcatgtttctcgcctgcgtcctggtcctgatcgtggacgccgtcctgcaactcagccccctcctgggagctgtgacagtggtctccatgaccctgctgctgctggccttcaacggaccccacgatcctctgccccaagatcctgacaataccgacgataacggcccccaagaccccgataacaccgacgacaatggccctcacgaccctctgccccatagcccttccgatagcgctggcaacgatggcggccctcctcagctgacagaggaggtggaaaataagggcggcgatcagggaccccccctgatgacagatggcggaggaggacacagccatgatagcggacatggcggaggcgatccccatctgcctaccctcctcctgggcagctccggttctggaggcgacgatgatgaccctcacggccctgtgcagctctcctactacgacggcaaaaggaccgaacaaggaaaagaggtcctggagaaggccaggggcagcacatacggaacccccaggcctcccatgtccgattggaccggaggagccctgctggtcctctacagcttcgccctgatgctgatcattatcatcctgatcatctttatcttcagaagggacctgctgtgccctctcggcgccctgtgcatcctgctgctcatgatcacactcctcctgatcgccctctggaacctgcacggacaagccctgatgtccgatgagggacctggaacaggacccggaaacggactgggcgagaagggagatacaagcggccccgaaggcagcggcggaagcggaccccaaagaaggggcggcgacaaccacggaagaggaagaggcaggggcagaggcagaggaggaggaagacctggagcccctggcggttctggaagcggacccaggcacagggacggagtgaggaggcctcaaaaaagacccagctgcatcggctgcaagggaacccactggattgatgataacccctccacagagaccgctcaggcctggaacgccggcttcctgaggggaagagcctatggcatcgatctgctgaggaccgagggcgaacacgtggagggagccaccggagagacaagggaggaaagcgaagacacagaaagcgatggcgacgacgaagacctgccctgcattgtgtccaggggcggacccaaggtgaagaggccccctatctttatcagaaggctccatagactgctcctgatgagggccatgaaccctgtgtgcctgcccgtgatcgtggccccctacctcttttggctggccgccattgccgctagctgcttcaccgcctccgtgtccacagtggtgacagccaccggcctcgccctgagcctgctgctcctcgctgccgtggcctccagctacgccgctgctcaaagaaagctcctgacccctgtcaccgtcctgacagccgtcgtgaccaccttttccgctggcaccttcaagctgcctaggtgcacacctggcgacaggcagtggctctacgtgcagagctccgtgggcaatattgtgcagagctgcaatcccaggtacagcatttttttcgactacatggccatccataggtccctcaccaagatctgggag

SEQ ID NO: 26 - protein, 1270 aa, synthetic construct
  REGION 1..1270: note = Description of Artificial Sequence: Synthetic polypeptide
MRPAPWTPNPPRSPSQMSVRDRLARLRAEAQVKQASVEVQPPQLTQVSPQQPVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTVRKPQQPESLEECDSELEIKRYKNRVASRKCRAKFKQLLQHYREVAAAKSSEIRDRRRNPASRRDQAKWRLQTLAAGWPMGYQAYSSWMYSYTDHQTTPTFVHLQATLGCTGGRRCHVFLGIVLFIFGCLLVLGIWIYLLEMLWRLGATIWQLLAFFLAFFLDLILLIIALYLQQNWWTLLVDLLWLLLFLAILIWMYYHGQRGRVACAPVPAPAGPIVRPWEPSLTQAAGQAFAPVRPQHMPVEPVPVPTVALERPVYPKPVRPVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIHSDEHHHDDSLPHPQQATDDSGHESDSNSNEGRHHLLVSGAQVPEPPTIHLAAQGMAYPLHEQHGMAPCPVAQAPPTPLPFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYDGKRTEQGKEVLEKARGSTYGTPRPPMSDWTGGALLVLYSFALMLIIIILIIFIFRRDLLCPLGALCILLLMITLLLIALWNLHGQALMSDEGPGTGPGNGLGEKGDTSGPEGSGGSGPQRRGGDNHGRGRGRGRGRGGGRPGAPGGSGSGPRHRDGVRRPQKRPSCIGCKGTHWIDDNPSTETAQAWNAGFLRGRAYGIDLLRTEGEHVEGATGETREESEDTESDGDDEDLPCIVSRGGPKVKRPPIFIRRLHRLLLMRAMNPVCLPVIVAPYLFWLAAIAASCFTASVSTVVTATGLALSLLLLAAVASSYAAAQRKLLTPVTVLTAVVTTFSAGTFKLPRCTPGDRQWLYVQSSVGNIVQSCNPRYSIFFDYMAIHRSLTKIWEDLGGPSQAPLPCVLWPVLPEPLPQGQLTAYHVSTAPTGSWFSAPQPAPENAYQAYAAPQLFPVSDI

SEQ ID NO: 27 - DNA, 3810 bp, synthetic construct
  misc_feature 1..3810: note = Description of Artificial Sequence: Synthetic polynucleotide
  source 1..3810: mol_type = other DNA
atgagacctgctccctggacacctaatcctcccaggtcccccagccagatgagcgtgagagacagactggctaggctgagagccgaggctcaggtcaagcaggccagcgtcgaggtgcaaccccctcagctcacccaggtgtccccccagcagcctgtggccggcattctgttcattctggccattctgaccgagtggggaagcggcaacagaacctacggccctgtcttcatgtgcctcggaggactgctgacaatggtggctggcgccgtgtggctcaccgtcatgtccaacaccctgctcagcgcctggattctgaccgccggattcctgatctttctgatcggattcgctctctttggcgtcatcaggtgttgcaggtactgttgctactactgcctgaccctcgagagcgaggaaagaccccccaccccctacaggaatacagtgaggaaacctcagcagcccgagagcctcgaggagtgcgatagcgagctggagattaaaaggtataagaatagggtggcctccaggaagtgtagggctaaattcaaacagctcctgcaacactatagggaagtggccgccgccaagtccagcgagattagggacagaaggaggaatcctgcctccaggagagaccaggccaaatggagactccaaacactcgccgctggatggcccatgggctaccaggcctatagctcctggatgtacagctacaccgaccatcagacaacacccaccttcgtgcatctgcaggctacactgggctgcaccggaggcagaaggtgtcacgtgtttctgggaatcgtgctgttcatctttggatgcctgctcgtgctgggcatctggatttatctcctggagatgctctggagactcggcgctacaatttggcagctgctcgccttttttctggccttctttctggacctgatcctcctgatcatcgccctgtacctccaacagaactggtggaccctcctggtggatctgctgtggctcctcctcttcctggccatcctgatctggatgtactaccatggccagagaggaagggtcgcttgcgctcctgtccctgctcctgctggccccatcgtgaggccttgggagccttccctcacacaggccgccggccaggcctttgctcccgtgaggccccagcacatgcctgtggaacccgtgcccgtccccacagtggctctggaaaggcctgtgtaccccaagcccgtgagacctgtcctctggctcagcagccctggaggactcggaacactcggagccgctctcctgacactggccgctgctctggctctgctggctagcctgatcctgggaaccctcaacctcaccaccatgtttctcctcatgctcctgtggaccctcgtggtgctgctcatctgttccagctgctccagctgccccctgagcaagatcctgctggccaggctgttcctgtacgccctcgccctcctgctgctggctagcgccctgatcgctggcggaagcatcctccagaccaatttcaagagcctctcctccaccgagttcatccccaacctgttctgtatgttactgctgatccatagcgacgagcaccatcatgacgactccctgccccatcctcagcaggccacagacgactccggccacgagagcgacagcaatagcaatgagggcaggcaccatctgctcgtgtccggagctcaagtccccgagcctcccaccatccatctcgccgcccagggaatggcttaccccctccacgagcagcacggcatggccccttgtcccgtcgctcaagccccccctacacctctgccctttttcgccatttgtctgacctggagaatcgaggacccccccttcaacagcctgctgttcgccctgctcgccgccgctggcggcctccagggcatttacgtcctcgtgatgctggtgctgctgatcctcgcttacaggagaagatggaggagactgacagtgtgcggcggcatcatgtttctcgcctgcgtcctggtcctgatcgtggacgccgtcctgcaactcagccccctcctgggagctgtgacagtggtctccatgaccctgctgctgctggccttcaacggaccccacgatcctctgccccaagatcctgacaataccgacgataacggcccccaagaccccgataacaccgacgacaatggccctcacgaccctctgccccatagcccttccgatagcgctggcaacgatggcggccctcctcagctgacagaggaggtggaaaataagggcggcgatcagggaccccccctgatgacagatggcggaggaggacacagccatgatagcggacatggcggaggcgatccccatctgcctaccctcctcctgggcagctccggttctggaggcgacgatgatgaccctcacggccctgtgcagctctcctactacgacggcaaaaggaccgaacaaggaaaagaggtcctggagaaggccaggggcagcacatacggaacccccaggcctcccatgtccgattggaccggaggagccctgctggtcctctacagcttcgccctgatgctgatcattatcatcctgatcatctttatcttcagaagggacctgctgtgccctctcggcgccctgtgcatcctgctgctcatgatcacactcctcctgatcgccctctggaacctgcacggacaagccctgatgtccgatgagggacctggaacaggacccggaaacggactgggcgagaagggagatacaagcggccccgaaggcagcggcggaagcggaccccaaagaaggggcggcgacaaccacggaagaggaagaggcaggggcagaggcagaggaggaggaagacctggagcccctggcggttctggaagcggacccaggcacagggacggagtgaggaggcctcaaaaaagacccagctgcatcggctgcaagggaacccactggattgatgataacccctccacagagaccgctcaggcctggaacgccggcttcctgaggggaagagcctatggcatcgatctgctgaggaccgagggcgaacacgtggagggagccaccggagagacaagggaggaaagcgaagacacagaaagcgatggcgacgacgaagacctgccctgcattgtgtccaggggcggacccaaggtgaagaggccccctatctttatcagaaggctccatagactgctcctgatgagggccatgaaccctgtgtgcctgcccgtgatcgtggccccctacctcttttggctggccgccattgccgctagctgcttcaccgcctccgtgtccacagtggtgacagccaccggcctcgccctgagcctgctgctcctcgctgccgtggcctccagctacgccgctgctcaaagaaagctcctgacccctgtcaccgtcctgacagccgtcgtgaccaccttttccgctggcaccttcaagctgcctaggtgcacacctggcgacaggcagtggctctacgtgcagagctccgtgggcaatattgtgcagagctgcaatcccaggtacagcatttttttcgactacatggccatccataggtccctcaccaagatctgggaggatctgggaggcccttcccaggctcctctgccctgcgtgctgtggcctgtgctgcctgagcctctgccccaaggccagctgacagcctatcacgtgtccaccgctcctacaggttcttggttcagcgctccccagcccgctcccgaaaacgcttaccaggcttacgccgccccccagctgttccccgtctccgacatc

SEQ ID NO: 28 - protein, 355 aa, unidentified
  source 1..355: note = Callitrichine gammaherpesvirus 3
MAPRRRLSGPPWLTVLLLLSTLSVAALLILFLIFSAGATISTEASLLVLLLLFVTLLLPLLSSNGLQLPAALILIQCFLLAADYLAYLILPTISEDFLILIAILVIVILVGTITTLVGAIGGIRARRSFLFICIFFLFLSLFLTILALLLGFSWLLLVAILFWVLWLVILILLLLVYPIPHHPLPTSLRFRMKQRVSSDPTGSDRSPQGSHNSLNSPDEEDPKDDTKQPLCNMTQGGPPVNGQLLGQHAQCPPHYPCCHIQHPDGEDSDGDDGKSWGDAGEEDNGPNDPNTNNGNEGGEGDDYKSWRKPEEEDNGPNDPNTNNRIEDGDGDDGKSWRNPEEEDNRKQDRLGTKPF

SEQ ID NO: 29 - protein, 93 aa, unidentified
  source 1..93: note = Callitrichine gammaherpesvirus 3
MAPRRRLSGPPWLTVLLLLSTLSVAALLILFLIFSAGATISTEASLLVLLLLFVTLLLPLLSSNGLQLPAALILIQCFLLAADYLAYLILPTI

SEQ ID NO: 30 - protein, 198 aa, unidentified
  source 1..198: note = Callitrichine gammaherpesvirus 3
SEDFLILIAILVIVILVGTITTLVGAIGGIRARRSFLFICIFFLFLSLFLTILALLLGFSWLLLVAILFWVLWLVILILLLLVYPIPHHPLPTSLRFRMKQRVSSDPTGSDRSPQGSHNSLNSPDEEDPKDDTKQPLCNMTQGGPPVNGQLLGQHAQCPPHYPCCHIQHPDGEDSDGDDGKSWGDAGEEDNGPNDPNT

SEQ ID NO: 31 - protein, 64 aa, unidentified
  source 1..64: note = Callitrichine gammaherpesvirus 3
NNGNEGGEGDDYKSWRKPEEEDNGPNDPNTNNRIEDGDGDDGKSWRNPEEEDNRKQDRLGTKPF

SEQ ID NO: 32 - protein, 413 aa, unidentified
  source 1..413: note = Callitrichine gammaherpesvirus 3
MAGHWYESVIPGLFLCPLILPSLFWICSLLTFLVGHGANIVSAVLFLVLAWCLLIANWNVTREDFVSGRRSSMSSLSVAASTATAMFASFLTLSFDGLGLLLFGTALVIQTIYVLYLVVMEITVWIMMFRYLHFWITLLFLLSPIILSVACLIIQSSALLIEAVVVTTITVLAIFLWLPPQGAEADLGTALLILNTALCLVVLILTAIPTDAQILTVFCLFCQWTLFICLGIRMICNWRGKLTRIICLKFCLYGLISASLSFGWYAFLKEVTLPTTATVDPRQLPLFLFILSSVLVILAIMMEFQTSSSLFAALFVIIAGMLCVTVGVIFLLAGVKPLLSGMICASGITMLVLGVVLLVVCTRASTRESIYEDLRYPTRDANGEYENVGYPPRDGDAPHRLGEPVYDDVEQAT

SEQ ID NO: 33 - protein, 210 aa, unidentified
  source 1..210: note = Callitrichine gammaherpesvirus 3
MAGHWYESVIPGLFLCPLILPSLFWICSLLTFLVGHGANIVSAVLFLVLAWCLLIANWNVTREDFVSGRRSSMSSLSVAASTATAMFASFLTLSFDGLGLLLFGTALVIQTIYVLYLVVMEITVWIMMFRYLHFWITLLFLLSPIILSVACLIIQSSALLIEAVVVTTITVLAIFLWLPPQGAEADLGTALLILNTALCLVVLILTAIPT

SEQ ID NO: 34 - protein, 153 aa, unidentified
  source 1..153: note = Callitrichine gammaherpesvirus 3
DAQILTVFCLFCQWTLFICLGIRMICNWRGKLTRIICLKFCLYGLISASLSFGWYAFLKEVTLPTTATVDPRQLPLFLFILSSVLVILAIMMEFQTSSSLFAALFVIIAGMLCVTVGVIFLLAGVKPLLSGMICASGITMLVLGVVLLVVCTR

SEQ ID NO: 35 - protein, 50 aa, unidentified
  source 1..50: note = Callitrichine gammaherpesvirus 3
ASTRESIYEDLRYPTRDANGEYENVGYPPRDGDAPHRLGEPVYDDVEQAT

SEQ ID NO: 36 - protein, 327 aa, unidentified
  source 1..327: note = Callitrichine gammaherpesvirus 3
MPRGRSTGRKGRDTEKERSRSPLRAPGGSDGPSTRAGCGAGPCQLSSPIAGGSRGGRGGRGGRGGSRGRGASRGRGGRGGRGGRGGRGGRGGRGGRGSPGDDSPSPCHHRDEPPSRSPSPQPTVSEQSQQSPRQQSPQGTSQGSTRPQVPGGATTRKRGGVRGQPAKCHGKYTTTAEGLTALLNRRHSPRTSNEGRWMNGVMAVNLSKWPLYSLRRALALAANEVRISPLFRLPYGSAFGPGPQPGPILESSTWGFLVFTQTSLFADDIADAIRDYCTTHPGPTRNTQVVLMNFEGSGVPLPMFFPPGEETEEQREGDRASDSDESE

SEQ ID NO: 37 - protein, 51 aa, unidentified
  source 1..51: note = Callitrichine gammaherpesvirus 3
MPRGRSTGRKGRDTEKERSRSPLRAPGGSDGPSTRAGCGAGPCQLSSPIAG

SEQ ID NO: 38 - protein, 119 aa, unidentified
  source 1..119: note = Callitrichine gammaherpesvirus 3
SPSPCHHRDEPPSRSPSPQPTVSEQSQQSPRQQSPQGTSQGSTRPQVPGGATTRKRGGVRGQPAKCHGKYTTTAEGLTALLNRRHSPRTSNEGRWMNGVMAVNLSKWPLYSLRRALALA

SEQ ID NO: 39 - protein, 106 aa, unidentified
  source 1..106: note = Callitrichine gammaherpesvirus 3
ANEVRISPLFRLPYGSAFGPGPQPGPILESSTWGFLVFTQTSLFADDIADAIRDYCTTHPGPTRNTQVVLMNFEGSGVPLPMFFPPGEETEEQREGDRASDSDESE

SEQ ID NO: 40 - protein, 252 aa, unidentified
  source 1..252: note = Callitrichine gammaherpesvirus 3
MDLDGTGGGEGYSQMVPIATAPGSGHAATYQDLQAAPYIIWPLQTDCQPVATTFASPGQIQWYTSAVPQPTEHCSQFTNAPTVNQQQPISQPQPENPPAFTFTQPASIIPGVISASNLNVSASPIIPSDHVLPIITSVTSLAQPNNDEHAISASHHASDGSVNQQKENQPQTLEECKTDQERKRYRNRLASRRCRAKFRNQLEHFRTVAAAKTEENNRLRVLIRQMCPTLDVESIVPSTSAGYHEPLNHLTH

SEQ ID NO: 41 - protein, 146 aa, unidentified
  source 1..146: note = Callitrichine gammaherpesvirus 3
MDLDGTGGGEGYSQMVPIATAPGSGHAATYQDLQAAPYIIWPLQTDCQPVATTFASPGQIQWYTSAVPQPTEHCSQFTNAPTVNQQQPISQPQPENPPAFTFTQPASIIPGVISASNLNVSASPIIPSDHVLPIITSVTSLAQPNN

SEQ ID NO: 42 - protein, 106 aa, unidentified
  source 1..106: note = Callitrichine gammaherpesvirus 3
DEHAISASHHASDGSVNQQKENQPQTLEECKTDQERKRYRNRLASRRCRAKFRNQLEHFRTVAAAKTEENNRLRVLIRQMCPTLDVESIVPSTSAGYHEPLNHLTH

SEQ ID NO: 43 - protein, 216 aa, Callithrix jacchus
MDDQRDLISNNEQLPMLGQRPGAPESKCSRGAVYTVFSILVALLLAGQATTAYFLYQQQGRLDKLTVTSQNLQLENLRMKLPKPAKPLSQMRMATPLLMQALPMAGLPQKPMQNATKHGNMTEDHVMHLLLNADPLKVYPPLKGSLSENLKHLKNTMETMDWKVFESWLHHWLLFEMSKHSLEQKPTEAPPKESLELEDPSSGLGVTKQDLGPVAM

SEQ ID NO: 44 - protein, 1045 aa, synthetic construct
  REGION 1..1045: note = Description of Artificial Sequence: Synthetic polypeptide
MSEDFLILIAILVIVILVGTITTLVGAIGGIRARRSFLFICIFFLFLSLFLTILALLLGFSWLLLVAILFWVLWLVILILLLLVYPIPHHPLPTSLRFRMKQRVSSDPTGSDRSPQGSHNSLNSPDEEDPKDDTKQPLCNMTQGGPPVNGQLLGQHAQCPPHYPCCHIQHPDGEDSDGDDGKSWGDAGEEDNGPNDPNTASTRESIYEDLRYPTRDANGEYENVGYPPRDGDAPHRLGEPVYDDVEQATANEVRISPLFRLPYGSAFGPGPQPGPILESSTWGFLVFTQTSLFADDIADAIRDYCTTHPGPTRNTQVVLMNFEGSGVPLPMFFPPGEETEEQREGDRASDSDESEDAQILTVFCLFCQWTLFICLGIRMICNWRGKLTRIICLKFCLYGLISASLSFGWYAFLKEVTLPTTATVDPRQLPLFLFILSSVLVILAIMMEFQTSSSLFAALFVIIAGMLCVTVGVIFLLAGVKPLLSGMICASGITMLVLGVVLLVVCTRSPSPCHHRDEPPSRSPSPQPTVSEQSQQSPRQQSPQGTSQGSTRPQVPGGATTRKRGGVRGQPAKCHGKYTTTAEGLTALLNRRHSPRTSNEGRWMNGVMAVNLSKWPLYSLRRALALAMAPRRRLSGPPWLTVLLLLSTLSVAALLILFLIFSAGATISTEASLLVLLLLFVTLLLPLLSSNGLQLPAALILIQCFLLAADYLAYLILPTIMPRGRSTGRKGRDTEKERSRSPLRAPGGSDGPSTRAGCGAGPCQLSSPIAGNNGNEGGEGDDYKSWRKPEEEDNGPNDPNTNNRIEDGDGDDGKSWRNPEEEDNRKQDRLGTKPFMAGHWYESVIPGLFLCPLILPSLFWICSLLTFLVGHGANIVSAVLFLVLAWCLLIANWNVTREDFVSGRRSSMSSLSVAASTATAMFASFLTLSFDGLGLLLFGTALVIQTIYVLYLVVMEITVWIMMFRYLHFWITLLFLLSPIILSVACLIIQSSALLIEAVVVTTITVLAIFLWLPPQGAEADLGTALLILNTALCLVVLILTAIPT

SEQ ID NO: 45 - DNA, 3135 bp, synthetic construct
  misc_feature 1..3135: note = Description of Artificial Sequence: Synthetic polynucleotide
  source 1..3135: mol_type = other DNA
atgtccgaggactttctgattctgatcgccatcctggtgatcgtgattctcgtgggcacaatcacaaccctggtgggcgccatcggcggcattagggccaggaggagcttcctcttcatttgcatcttcttcctgttcctctccctcttcctgacaatcctcgccctgctgctgggcttcagctggctcctgctggtggccatcctgttctgggtgctctggctggtcatcctcattctgctgctgctggtgtaccctattcctcaccaccccctgcccacctccctcaggtttagaatgaagcagagggtgagcagcgaccccacaggttctgacagaagccctcagggcagccataatagcctgaactcccccgatgaggaggaccccaaggatgacaccaagcaacctctgtgcaacatgacccagggcggacctcccgtcaatggacagctcctcggacaacatgctcaatgcccccctcactatccctgctgccatattcagcatcccgacggagaggattccgatggagacgatggcaagtcctggggcgatgccggagaggaagacaatggccctaacgaccctaacaccgccagcaccagagagtccatttacgaggacctcagataccccacaagggacgccaatggcgagtatgagaacgtgggatacccccctagggacggagatgcccctcataggctcggagagcctgtgtatgacgatgtggagcaagccaccgctaacgaggtgagaatctcccctctgttcagactgccctacggaagcgctttcggacctggcccccagcctggacccattctggagagctccacatggggctttctggtcttcacacagacctccctgttcgccgacgacattgccgacgctattagggactactgcacaacccaccctggccccacaaggaacacccaggtggtcctcatgaacttcgagggcagcggagtgcccctgcctatgttttttccccctggagaggagacagaagagcagagagagggcgatagagctagcgactccgacgagtccgaagacgctcagatcctgaccgtgttctgcctgttttgccagtggacactctttatctgcctgggaatcaggatgatctgtaactggaggggcaaactcaccaggatcatctgcctgaagttctgcctctacggactgatttccgcctccctgtccttcggctggtacgcttttctgaaggaagtgaccctccccaccacagccaccgttgatcctaggcaactccccctgttcctcttcatcctgagctccgtgctggtgattctcgccatcatgatggagtttcaaacatcctccagcctcttcgctgctctgttcgtgattatcgccggaatgctgtgcgtcacagtgggcgtgatttttctgctggctggcgtcaagcctctcctgagcggcatgatctgcgcctccggcatcacaatgctcgtgctcggcgtcgtgctgctggtggtgtgcaccagaagccccagcccttgtcatcacagggatgaacccccctccagaagccccagccctcaacccaccgtctccgagcagtcccagcagtcccccaggcagcagagccctcaaggcacatcccagggttctacaagacctcaggtgcctggaggcgccaccaccagaaaaagaggcggcgtgagaggccaacctgccaagtgtcacggcaagtacaccacaaccgccgagggactgaccgctctcctgaataggaggcacagccccaggacatccaacgagggcaggtggatgaatggagtcatggctgtgaacctctccaaatggcccctgtacagcctgaggagagccctggccctcgccatggctcctagaaggaggctctccggccctccctggctgacagtgctgctgctgctgtccacactgagcgtggccgccctgctgattctcttcctgattttcagcgccggcgccaccattagcacagaagccagcctgctggtcctgctcctgctgtttgtgaccctgctgctgcctctcctgtcctccaacggactccagctccctgccgccctgattctgatccagtgtttcctcctggccgctgattatctcgcctacctgattctgcctaccattatgcccaggggcagaagcacaggaaggaagggcagggacacagagaaagagaggagcagatcccctctcagagctcctggcggttctgatggacccagcacaagggctggctgtggagccggaccctgtcagctgagcagccccatcgccggaaacaacggcaatgaaggcggcgagggcgacgactacaagagctggaggaagcccgaggaagaggacaacggccccaatgaccccaataccaacaacaggattgaggatggagacggcgacgacggaaaatcctggaggaatcctgaggaggaggataacagaaagcaggacaggctgggcaccaagcctttcatggccggccactggtatgagagcgtgattcccggcctgttcctctgccccctgatcctcccttccctgttctggatttgctccctgctgaccttcctggtgggccacggagccaatattgtgagcgccgtcctgttcctcgtgctggcttggtgtctcctcattgccaactggaacgtgacaagagaggacttcgtgtccggcaggagaagctccatgagcagcctgtccgtggccgcttccaccgccacagccatgttcgccagcttcctcaccctgagctttgatggcctgggcctgctgctgtttggcaccgccctggtgatccagacaatttacgtgctgtatctggtggtcatggagatcaccgtgtggatcatgatgtttaggtatctccacttttggatcaccctgctgttcctgctgagccccattattctctccgtcgcctgtctcatcatccaatcctccgccctgctgatcgaggctgtggtcgtcaccaccatcacagtcctggccatttttctgtggctccctcctcaaggcgctgaggccgatctcggcaccgccctgctgattctgaataccgccctgtgcctggtcgtgctgatcctgaccgctatccctaca

SEQ ID NO: 46 - protein, 1270 aa, synthetic construct; identical to SEQ ID NO: 26
  REGION 1..1270: note = Description of Artificial Sequence: Synthetic polypeptide

SEQ ID NO: 47 - DNA, 3810 bp, synthetic construct; identical to SEQ ID NO: 27
  misc_feature 1..3810: note = Description of Artificial Sequence: Synthetic polynucleotide
  source 1..3810: mol_type = other DNA

SEQ ID NO: 48 - protein, 1512 aa, synthetic construct
  REGION 1..1512: note = Description of Artificial Sequence: Synthetic polypeptide
MDDQRDLISNNEQLPMLGQRPGAPESKCSRGAVYTVFSILVALLLAGQATTAYFLYQQQGRLDKLTVTSQNLQLENLRMKLPKPAKPLSQMRMATPLLMQALPMAGLPQKPMQNATKHGNMTEDHVMHLLLNADPLKVYPPLKGSLSENLKHLKNTMETMDWKVFESWLHHWLLFEMSKHSLEQKPTEAPPKESLELEDPSSGLGVTKQDLGPVAMSEDFLILIAILVIVILVGTITTLVGAIGGIRARRSFLFICIFFLFLSLFLTILALLLGFSWLLLVAILFWVLWLVILILLLLVYPIPHHPLPTSLRFRMKQRVSSDPTGSDRSPQGSHNSLNSPDEEDPKDDTKQPLCNMTQGGPPVNGQLLGQHAQCPPHYPCCHIQHPDGEDSDGDDGKSWGDAGEEDNGPNDPNTASTRESIYEDLRYPTRDANGEYENVGYPPRDGDAPHRLGEPVYDDVEQATANEVRISPLFRLPYGSAFGPGPQPGPILESSTWGFLVFTQTSLFADDIADAIRDYCTTHPGPTRNTQVVLMNFEGSGVPLPMFFPPGEETEEQREGDRASDSDESEDAQILTVFCLFCQWTLFICLGIRMICNWRGKLTRIICLKFCLYGLISASLSFGWYAFLKEVTLPTTATVDPRQLPLFLFILSSVLVILAIMMEFQTSSSLFAALFVIIAGMLCVTVGVIFLLAGVKPLLSGMICASGITMLVLGVVLLVVCTRDEHAISASHHASDGSVNQQKENQPQTLEECKTDQERKRYRNRLASRRCRAKFRNQLEHFRTVAAAKTEENNRLRVLIRQMCPTLDVESIVPSTSAGYHEPLNHLTHSPSPCHHRDEPPSRSPSPQPTVSEQSQQSPRQQSPQGTSQGSTRPQVPGGATTRKRGGVRGQPAKCHGKYTTTAEGLTALLNRRHSPRTSNEGRWMNGVMAVNLSKWPLYSLRRALALAMAPRRRLSGPPWLTVLLLLSTLSVAALLILFLIFSAGATISTEASLLVLLLLFVTLLLPLLSSNGLQLPAALILIQCFLLAADYLAYLILPTIMPRGRSTGRKGRDTEKERSRSPLRAPGGSDGPSTRAGCGAGPCQLSSPIAGNNGNEGGEGDDYKSWRKPEEEDNGPNDPNTNNRIEDGDGDDGKSWRNPEEEDNRKQDRLGTKPFMDLDGTGGGEGYSQMVPIATAPGSGHAATYQDLQAAPYIIWPLQTDCQPVATTFASPGQIQWYTSAVPQPTEHCSQFTNAPTVNQQQPISQPQPENPPAFTFTQPASIIPGVISASNLNVSASPIIPSDHVLPIITSVTSLAQPNNMAGHWYESVIPGLFLCPLILPSLFWICSLLTFLVGHGANIVSAVLFLVLAWCLLIANWNVTREDFVSGRRSSMSSLSVAASTATAMFASFLTLSFDGLGLLLFGTALVIQTIYVLYLVVMEITVWIMMFRYLHFWITLLFLLSPIILSVACLIIQSSALLIEAVVVTTITVLAIFLWLPPQGAEADLGTALLILNTALCLVVLILTAIPT

SEQ ID NO: 49 - DNA, 4536 bp, synthetic construct
  misc_feature 1..4536: note = Description of Artificial Sequence: Synthetic polynucleotide
  source 1..4536: mol_type = other DNA
atggacgaccagcgggacctgatcagcaacaacgagcagctgcccatgctgggccagaggcctggcgcccctgagagcaagtgtagcagaggcgccgtgtacaccgtgttcagcatcctggtggccctgctgctggccggacaggccaccaccgcctactttctgtatcagcagcagggacggctggacaagctgaccgtgaccagccagaacctgcagctggaaaacctgcggatgaagctgcccaagcccgccaagcccctgagccagatgagaatggccacccccctgctgatgcaggccctgcctatggccggcctgccccagaaacccatgcagaacgccaccaagcacggcaacatgaccgaggaccacgtgatgcatctgctgctgaacgccgaccccctgaaggtgtaccccccactgaagggcagcctgagcgagaacctgaagcacctgaagaacaccatggaaaccatggactggaaggtgttcgagagctggctgcaccactggctgctgttcgagatgagcaagcacagcctggaacagaagcccaccgaggcccctcccaaagagagcctggaactggaagatcccagcagcggcctgggcgtgaccaagcaggatctgggccccgtggctatgtccgaggactttctgattctgatcgccatcctggtgatcgtgattctcgtgggcacaatcacaaccctggtgggcgccatcggcggcattagggccaggaggagcttcctcttcatttgcatcttcttcctgttcctctccctcttcctgacaatcctcgccctgctgctgggcttcagctggctcctgctggtggccatcctgttctgggtgctctggctggtcatcctcattctgctgctgctggtgtaccctattcctcaccaccccctgcccacctccctcaggtttagaatgaagcagagggtgagcagcgaccccacaggttctgacagaagccctcagggcagccataatagcctgaactcccccgatgaggaggaccccaaggatgacaccaagcaacctctgtgcaacatgacccagggcggacctcccgtcaatggacagctcctcggacaacatgctcaatgcccccctcactatccctgctgccatattcagcatcccgacggagaggattccgatggagacgatggcaagtcctggggcgatgccggagaggaagacaatggccctaacgaccctaacaccgccagcaccagagagtccatttacgaggacctcagataccccacaagggacgccaatggcgagtatgagaacgtgggatacccccctagggacggagatgcccctcataggctcggagagcctgtgtatgacgatgtggagcaagccaccgctaacgaggtgagaatctcccctctgttcagactgccctacggaagcgctttcggacctggcccccagcctggacccattctggagagctccacatggggctttctggtcttcacacagacctccctgttcgccgacgacattgccgacgctattagggactactgcacaacccaccctggccccacaaggaacacccaggtggtcctcatgaacttcgagggcagcggagtgcccctgcctatgttttttccccctggagaggagacagaagagcagagagagggcgatagagctagcgactccgacgagtccgaagacgctcagatcctgaccgtgttctgcctgttttgccagtggacactctttatctgcctgggaatcaggatgatctgtaactggaggggcaaactcaccaggatcatctgcctgaagttctgcctctacggactgatttccgcctccctgtccttcggctggtacgcttttctgaaggaagtgaccctccccaccacagccaccgttgatcctaggcaactccccctgttcctcttcatcctgagctccgtgctggtgattctcgccatcatgatggagtttcaaacatcctccagcctcttcgctgctctgttcgtgattatcgccggaatgctgtgcgtcacagtgggcgtgatttttctgctggctggcgtcaagcctctcctgagcggcatgatctgcgcctccggcatcacaatgctcgtgctcggcgtcgtgctgctggtggtgtgcaccagagatgagcacgctatttccgccagccaccatgctagcgatggctccgtgaatcagcagaaggaaaatcagccccagaccctggaggaatgcaagacagatcaggagaggaagaggtacaggaacaggctggcctccaggaggtgtagagctaagttcaggaaccagctggaacattttaggacagtcgccgctgctaagacagaggagaacaacaggctcagggtgctcatcaggcagatgtgtcctacactggacgtggaatccatcgtcccctccacctccgccggctaccacgagcctctgaatcacctgacccacagccccagcccttgtcatcacagggatgaacccccctccagaagccccagccctcaacccaccgtctccgagcagtcccagcagtcccccaggcagcagagccctcaaggcacatcccagggttctacaagacctcaggtgcctggaggcgccaccaccagaaaaagaggcggcgtgagaggccaacctgccaagtgtcacggcaagtacaccacaaccgccgagggactgaccgctctcctgaataggaggcacagccccaggacatccaacgagggcaggtggatgaatggagtcatggctgtgaacctctccaaatggcccctgtacagcctgaggagagccctggccctcgccatggctcctagaaggaggctctccggccctccctggctgacagtgctgctgctgctgtccacactgagcgtggccgccctgctgattctcttcctgattttcagcgccggcgccaccattagcacagaagccagcctgctggtcctgctcctgctgtttgtgaccctgctgctgcctctcctgtcctccaacggactccagctccctgccgccctgattctgatccagtgtttcctcctggccgctgattatctcgcctacctgattctgcctaccattatgcccaggggcagaagcacaggaaggaagggcagggacacagagaaagagaggagcagatcccctctcagagctcctggcggttctgatggacccagcacaagggctggctgtggagccggaccctgtcagctgagcagccccatcgccggaaacaacggcaatgaaggcggcgagggcgacgactacaagagctggaggaagcccgaggaagaggacaacggccccaatgaccccaataccaacaacaggattgaggatggagacggcgacgacggaaaatcctggaggaatcctgaggaggaggataacagaaagcaggacaggctgggcaccaagcctttcatggacctcgacggaaccggcggaggcgagggctacagccagatggtccctatcgccaccgcccccggaagcggccacgccgctacctatcaggatctccaggccgccccttacatcatctggcctctccagaccgattgccagcctgtggctaccaccttcgcctcccccggacagatccagtggtatacaagcgccgtcccccagcccacagagcattgctcccagtttacaaacgctcccaccgtcaaccagcagcagcctattagccaaccccagcccgaaaatccccctgctttcacctttacccagcccgcttccatcattcccggcgtcattagcgcctccaacctgaacgtgagcgcttcccctatcatccctagcgaccatgtcctccccatcattacctccgtgaccagcctcgcccaacctaataacatggccggccactggtatgagagcgtgattcccggcctgttcctctgccccctgatcctcccttccctgttctggatttgctccctgctgaccttcctggtgggccacggagccaatattgtgagcgccgtcctgttcctcgtgctggcttggtgtctcctcattgccaactggaacgtgacaagagaggacttcgtgtccggcaggagaagctccatgagcagcctgtccgtggccgcttccaccgccacagccatgttcgccagcttcctcaccctgagctttgatggcctgggcctgctgctgtttggcaccgccctggtgatccagacaatttacgtgctgtatctggtggtcatggagatcaccgtgtggatcatgatgtttaggtatctccacttttggatcaccctgctgttcctgctgagccccattattctctccgtcgcctgtctcatcatccaatcctccgccctgctgatcgaggctgtggtcgtcaccaccatcacagtcctggccatttttctgtggctccctcctcaaggcgctgaggccgatctcggcaccgccctgctgattctgaataccgccctgtgcctggtcgtgctgatcctgaccgctatccctaca

SEQ ID NO: 50 - DNA, 44035 bp, synthetic construct
  misc_difference 15712: note = modified_base - a, c, t, g, unknown or other
  misc_feature 1..44035: note = Description of Artificial Sequence: Synthetic polynucleotide
  source 1..44035: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatgagacctgctccctggacacctaatcctcccaggtcccccagccagatgagcgtgagagacagactggctaggctgagagccgaggctcaggtcaagcaggccagcgtcgaggtgcaaccccctcagctcacccaggtgtccccccagcagcctgtggccggcattctgttcattctggccattctgaccgagtggggaagcggcaacagaacctacggccctgtcttcatgtgcctcggaggactgctgacaatggtggctggcgccgtgtggctcaccgtcatgtccaacaccctgctcagcgcctggattctgaccgccggattcctgatctttctgatcggattcgctctctttggcgtcatcaggtgttgcaggtactgttgctactactgcctgaccctcgagagcgaggaaagaccccccaccccctacaggaatacagtgattagggacagaaggaggaatcctgcctccaggagagaccaggccaaatggagactccaaacactcgccgctggatggcccatgggctaccaggcctatagctcctggatgtacagctacaccgaccatcagacaacacccaccttcgtgcatctgcaggctacactgggctgcaccggaggcagaaggtgtcacgtgtttctgggaatcgtgctgttcatctttggatgcctgctcgtgctgggcatctggatttatctcctggagatgctctggagactcggcgctacaatttggcagctgctcgccttttttctggccttctttctggacctgatcctcctgatcatcgccctgtacctccaacagaactggtggaccctcctggtggatctgctgtggctcctcctcttcctggccatcctgatctggatgtactaccatggccagagaggaagggtcgcttgcgctcctgtccctgctcctgctggccccatcgtgaggccttgggagccttccctcacacaggccgccggccaggcctttgctcccgtgaggccccagcacatgcctgtggaacccgtgcccgtccccacagtggctctggaaaggcctgtgtaccccaagcccgtgagacctgtcctctggctcagcagccctggaggactcggaacactcggagccgctctcctgacactggccgctgctctggctctgctggctagcctgatcctgggaaccctcaacctcaccaccatgtttctcctcatgctcctgtggaccctcgtggtgctgctcatctgttccagctgctccagctgccccctgagcaagatcctgctggccaggctgttcctgtacgccctcgccctcctgctgctggctagcgccctgatcgctggcggaagcatcctccagaccaatttcaagagcctctcctccaccgagttcatccccaacctgttctgtatgttactgctgatccatagcgacgagcaccatcatgacgactccctgccccatcctcagcaggccacagacgactccggccacgagagcgacagcaatagcaatgagggcaggcaccatctgctcgtgtccggagctcaagtccccgagcctcccaccatccatctcgccgcccagggaatggcttaccccctccacgagcagcacggcatggccccttgtcccgtcgctcaagccccccctacacctctgccctttttcgccatttgtctgacctggagaatcgaggacccccccttcaacagcctgctgttcgccctgctcgccgccgctggcggcctccagggcatttacgtcctcgtgatgctggtgctgctgatcctcgcttacaggagaagatggaggagactgacagtgtgcggcggcatcatgtttctcgcctgcgtcctggtcctgatcgtggacgccgtcctgcaactcagccccctcctgggagctgtgacagtggtctccatgaccctgctgctgctggccttcaacggaccccacgatcctctgccccaagatcctgacaataccgacgataacggcccccaagaccccgataacaccgacgacaatggccctcacgaccctctgccccatagcccttccgatagcgctggcaacgatggcggccctcctcagctgacagaggaggtggaaaataagggcggcgatcagggaccccccctgatgacagatggcggaggaggacacagccatgatagcggacatggcggaggcgatccccatctgcctaccctcctcctgggcagctccggttctggaggcgacgatgatgaccctcacggccctgtgcagctctcctactacgacggcaaaaggaccgaacaaggaaaagaggtcctggagaaggccaggggcagcacatacggaacccccaggcctcccatgtccgattggaccggaggagccctgctggtcctctacagcttcgccctgatgctgatcattatcatcctgatcatctttatcttcagaagggacctgctgtgccctctcggcgccctgtgcatcctgctgctcatgatcacactcctcctgatcgccctctggaacctgcacggacaagccctgatgtccgatgagggacctggaacaggacccggaaacggactgggcgagaagggagatacaagcggccccgaaggcagcggcggaagcggaccccaaagaaggggcggcgacaaccacggaagaggaagaggcaggggcagaggcagaggaggaggaagacctggagcccctggcggttctggaagcggacccaggcacagggacggagtgaggaggcctcaaaaaagacccagctgcatcggctgcaagggaacccactggattgatgataacccctccacagagaccgctcaggcctggaacgccggcttcctgaggggaagagcctatggcatcgatctgctgaggaccgagggcgaacacgtggagggagccaccggagagacaagggaggaaagcgaagacacagaaagcgatggcgacgacgaagacctgccctgcattgtgtccaggggcggacccaaggtgaagaggccccctatctttatcagaaggctccatagactgctcctgatgagggccatgaaccctgtgtgcctgcccgtgatcgtggccccctacctcttttggctggccgccattgccgctagctgcttcaccgcctccgtgtccacagtggtgacagccaccggcctcgccctgagcctgctgctcctcgctgccgtggcctccagctacgccgctgctcaaagaaagctcctgacccctgtcaccgtcctgacagccgtcgtgaccaccttttccgctggcaccttcaagctgcctaggtgcacacctggcgacaggcagtggctctacgtgcagagctccgtgggcaatattgtgcagagctgcaatcccaggtacagcatttttttcgactacatggccatccataggtccctcaccaagatctgggagtgatgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaangcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactggggctcctgtcgccatcctgtgaacgccaccgtcttcacccaccccgaccaggcccaggcgaacctcacctgcggtctgcatcggagggccaagaagtacctcacctggtacttcaacggcaccccctttgtggtttacaacagcttcgacggggacggagtctccctgaaagaccagctctccggtctcagctactccatccacaagaacaccaccctccaactcttccctccctacctgccgggaacctacgagtgcgtcaccggccgctgcacccacctcacccgcctgatcgtaaaccagagctttccgggaacagataactccctcttccccagaacaggaggtgagctcaggaaactccccggggaccagggcggagacgtaccttcgacccttgtggggttaggattttttattaccgggttgctggctcttttaatcaaagtttccttgagatttgttctttccttctacgtgtatgaacacctcaacctccaataactctaccctttcttcggaatcaggtgacttctctgaaatcgggcttggtgtgctgcttactctgttgatttttttccttatcatactcagccttctgtgcctcaggctcgccgcctgctgcgcacacatctatatctactgctggttgctcaagtgcaggggtcgccacccaagatgaacaggtacatggtcctatcgatcctaggcctgctggccctggcggcctgcagcgccgccaaaaaagagattacctttgaggagcccgcttgcaatgtaactttcaagcccgagggtgaccaatgcaccaccctcgtcaaatgcgttaccaatcatgagaggctgcgcatcgactacaaaaacaaaactggccagtttgcggtctatagtgtgtttacgcccggagacccctctaactactctgtcaccgtcttccagggcggacagtctaagatattcaattacactttccctttttatgagttatgcgatgcggtcatgtacatgtcaaaacagtacaacctgtggcctccctctccccaggcgtgtgtggaaaatactgggtcttactgctgtatggctttcgcaatcactacgctcgctctaatctgcacggtgctatacataaaattcaggcagaggcgaatctttatcgatgaaaagaaaatgccttgatcgctaacaccggctttctatctgcagaatgaatgcaatcacctccctactaatcaccaccaccctccttgcgattgcccatgggttgacacgaatcgaagtgccagtggggtccaatgtcaccatggtgggccccgccggcaattccaccctcatgtgggaaaaatttgtccgcaatcaatgggttcatttctgctctaaccgaatcagtatcaagcccagagccatctgcgatgggcaaaatctaactctgatcaatgtgcaaatgatggatgctgggtactattacgggcagcggggagaaatcattaattactggcgaccccacaaggactacatgctgcatgtagtcgaggcacttcccactaccacccccactaccacctctcccaccaccaccaccactactactactactactactactactactactaccactaccgctgcccgccatacccgcaaaagcaccatgattagcacaaagccccctcgtgctcactcccacgccggcgggcccatcggtgcgacctcagaaaccaccgagctttgcttctgccaatgcactaacgccagcgctcatgaactgttcgacctggagaatgaggatgtccagcagagctccgcttgcctgacccaggaggctgtggagcccgttgccctgaagcagatcggtgattcaataattgactcttcttcttttgccactcccgaataccctcccgattctactttccacatcacgggtaccaaagaccctaacctctctttctacctgatgctgctgctctgtatctctgtggtctcttccgcgctgatgttactggggatgttctgctgcctgatctgccgcagaaagagaaaagctcgctctcagggccaaccactgatgcccttcccctaccccccggattttgcagataacaagatatgagctcgctgctgacactaaccgctttactagcctgcgctctaacccttgtcgcttgcgactcgagattccacaatgtcacagctgtggcaggagaaaatgttactttcaactccacggccgatacccagtggtcgtggagtggctcaggtagctacttaactatctgcaatagctccacttcccccggcatatccccaaccaagtaccaatgcaatgccagcctgttcaccctcatcaacgcttccaccctggacaatggactctatgtaggctatgtaccctttggtgggcaaggaaagacccacgcttacaacctggaagttcgccagcccagaaccactacccaagcttctcccaccaccaccaccaccaccaccatcaccagcagcagcagcagcagcagccacagcagcagcagcagattattgactttggttttggccagctcatctgccgctacccaggccatctacagctctgtgcccgaaaccactcagatccaccgcccagaaacgaccaccgccaccaccctacacacctccagcgatcagatgccgaccaacatcacccccttggctcttcaaatgggacttacaagccccactccaaaaccagtggatgcggccgaggtctccgccctcgtcaatgactgggcggggctgggaatgtggtggttcgccataggcatgatggcgctctgcctgcttctgctctggctcatctgctgcctccaccgcaggcgagccagaccccccatctatagacccatcattgtcctgaaccccgataatgatgggatccatagattggatggcctgaaaaacctacttttttcttttacagtatgataaattgagacatgcctcgcattttcttgtacatgttccttctcccaccttttctggggtgttctacgctggccgctgtgtctcacctggaggtagactgcctctcacccttcactgtctacctgctttacggattggtcaccctcactctcatctgcagcctaatcacagtaatcatcgccttcatccagtgcattgattacatctgtgtgcgcctcgcatacttcagacaccacccgcagtaccgagacaggaacattgcccaacttctaagactgctctaatcatgcataagactgtgatctgccttctgatcctctgcatcctgcccaccctcacctcctgccagtacaccacaaaatctccgcgcaaaagacatgcctcctgccgcttcacccaactgtggaatatacccaaatgctacaacgaaaagagcgagctctccgaagcttggctgtatggggtcatctgtgtcttagttttctgcagcactgtctttgccctcataatctacccctactttgatttgggatggaacgcgatcgatgccatgaattaccccacctttcccgcacccgagataattccactgcgacaagttgtacccgttgtcgttaatcaacgccccccatcccctacgcccactgaaatcagctactttaacctaacaggcggagatgactgacgccctagatctagaaatggacggcatcagtaccgagcagcgtctcctagagaggcgcaggcaggcggctgagcaagagcgcctcaatcaggagctccgagatctcgttaacctgcaccagtgcaaaagaggcatcttttgtctggtaaagcaggccaaagtcacctacgagaagaccggcaacagccaccgcctcagttacaaattgcccacccagcgccagaagctggtgctcatggtgggtgagaatcccatcaccgtcacccagcactcggtagagaccgaggggtgtctgcactccccctgtcggggtccagaagacctctgcaccctggtaaagaccctgtgcggtctcagagatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatggtttaaacggatcctctagagtcgacctgcaggcatgcaagcttgagtataacccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgctcggtacccggggatcctcgtttaaac

SEQ ID NO: 51 - DNA, 41464 bp, synthetic construct
  misc_difference 16063: note = modified_base - a, c, t, g, unknown or other
  misc_feature 1..41464: note = Description of Artificial Sequence: Synthetic polynucleotide
  source 1..41464: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatgagacctgctccctggacacctaatcctcccaggtcccccagccagatgagcgtgagagacagactggctaggctgagagccgaggctcaggtcaagcaggccagcgtcgaggtgcaaccccctcagctcacccaggtgtccccccagcagcctgtggccggcattctgttcattctggccattctgaccgagtggggaagcggcaacagaacctacggccctgtcttcatgtgcctcggaggactgctgacaatggtggctggcgccgtgtggctcaccgtcatgtccaacaccctgctcagcgcctggattctgaccgccggattcctgatctttctgatcggattcgctctctttggcgtcatcaggtgttgcaggtactgttgctactactgcctgaccctcgagagcgaggaaagaccccccaccccctacaggaatacagtgaggaaacctcagcagcccgagagcctcgaggagtgcgatagcgagctggagattaaaaggtataagaatagggtggcctccaggaagtgtagggctaaattcaaacagctcctgcaacactatagggaagtggccgccgccaagtccagcgagattagggacagaaggaggaatcctgcctccaggagagaccaggccaaatggagactccaaacactcgccgctggatggcccatgggctaccaggcctatagctcctggatgtacagctacaccgaccatcagacaacacccaccttcgtgcatctgcaggctacactgggctgcaccggaggcagaaggtgtcacgtgtttctgggaatcgtgctgttcatctttggatgcctgctcgtgctgggcatctggatttatctcctggagatgctctggagactcggcgctacaatttggcagctgctcgccttttttctggccttctttctggacctgatcctcctgatcatcgccctgtacctccaacagaactggtggaccctcctggtggatctgctgtggctcctcctcttcctggccatcctgatctggatgtactaccatggccagagaggaagggtcgcttgcgctcctgtccctgctcctgctggccccatcgtgaggccttgggagccttccctcacacaggccgccggccaggcctttgctcccgtgaggccccagcacatgcctgtggaacccgtgcccgtccccacagtggctctggaaaggcctgtgtaccccaagcccgtgagacctgtcctctggctcagcagccctggaggactcggaacactcggagccgctctcctgacactggccgctgctctggctctgctggctagcctgatcctgggaaccctcaacctcaccaccatgtttctcctcatgctcctgtggaccctcgtggtgctgctcatctgttccagctgctccagctgccccctgagcaagatcctgctggccaggctgttcctgtacgccctcgccctcctgctgctggctagcgccctgatcgctggcggaagcatcctccagaccaatttcaagagcctctcctccaccgagttcatccccaacctgttctgtatgttactgctgatccatagcgacgagcaccatcatgacgactccctgccccatcctcagcaggccacagacgactccggccacgagagcgacagcaatagcaatgagggcaggcaccatctgctcgtgtccggagctcaagtccccgagcctcccaccatccatctcgccgcccagggaatggcttaccccctccacgagcagcacggcatggccccttgtcccgtcgctcaagccccccctacacctctgccctttttcgccatttgtctgacctggagaatcgaggacccccccttcaacagcctgctgttcgccctgctcgccgccgctggcggcctccagggcatttacgtcctcgtgatgctggtgctgctgatcctcgcttacaggagaagatggaggagactgacagtgtgcggcggcatcatgtttctcgcctgcgtcctggtcctgatcgtggacgccgtcctgcaactcagccccctcctgggagctgtgacagtggtctccatgaccctgctgctgctggccttcaacggaccccacgatcctctgccccaagatcctgacaataccgacgataacggcccccaagaccccgataacaccgacgacaatggccctcacgaccctctgccccatagcccttccgatagcgctggcaacgatggcggccctcctcagctgacagaggaggtggaaaataagggcggcgatcagggaccccccctgatgacagatggcggaggaggacacagccatgatagcggacatggcggaggcgatccccatctgcctaccctcctcctgggcagctccggttctggaggcgacgatgatgaccctcacggccctgtgcagctctcctactacgacggcaaaaggaccgaacaaggaaaagaggtcctggagaaggccaggggcagcacatacggaacccccaggcctcccatgtccgattggaccggaggagccctgctggtcctctacagcttcgccctgatgctgatcattatcatcctgatcatctttatcttcagaagggacctgctgtgccctctcggcgccctgtgcatcctgctgctcatgatcacactcctcctgatcgccctctggaacctgcacggacaagccctgatgtccgatgagggacctggaacaggacccggaaacggactgggcgagaagggagatacaagcggccccgaaggcagcggcggaagcggaccccaaagaaggggcggcgacaaccacggaagaggaagaggcaggggcagaggcagaggaggaggaagacctggagcccctggcggttctggaagcggacccaggcacagggacggagtgaggaggcctcaaaaaagacccagctgcatcggctgcaagggaacccactggattgatgataacccctccacagagaccgctcaggcctggaacgccggcttcctgaggggaagagcctatggcatcgatctgctgaggaccgagggcgaacacgtggagggagccaccggagagacaagggaggaaagcgaagacacagaaagcgatggcgacgacgaagacctgccctgcattgtgtccaggggcggacccaaggtgaagaggccccctatctttatcagaaggctccatagactgctcctgatgagggccatgaaccctgtgtgcctgcccgtgatcgtggccccctacctcttttggctggccgccattgccgctagctgcttcaccgcctccgtgtccacagtggtgacagccaccggcctcgccctgagcctgctgctcctcgctgccgtggcctccagctacgccgctgctcaaagaaagctcctgacccctgtcaccgtcctgacagccgtcgtgaccaccttttccgctggcaccttcaagctgcctaggtgcacacctggcgacaggcagtggctctacgtgcagagctccgtgggcaatattgtgcagagctgcaatcccaggtacagcatttttttcgactacatggccatccataggtccctcaccaagatctgggaggatctgggaggcccttcccaggctcctctgccctgcgtgctgtggcctgtgctgcctgagcctctgccccaaggccagctgacagcctatcacgtgtccaccgctcctacaggttcttggttcagcgctccccagcccgctcccgaaaacgcttaccaggcttacgccgccccccagctgttccccgtctccgacatctgatgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaangcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactgggatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatggtttaaacggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctcgtttaaac

SEQ ID NO: 52 - DNA, 43711 bp, synthetic construct
  misc_difference 15388: note = modified_base - a, c, t, g, unknown or other
  misc_feature 1..43711: note = Description of Artificial Sequence: Synthetic polynucleotide
  source 1..43711: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatgtccgaggactttctgattctgatcgccatcctggtgatcgtgattctcgtgggcacaatcacaaccctggtgggcgccatcggcggcattagggccaggaggagcttcctcttcatttgcatcttcttcctgttcctctccctcttcctgacaatcctcgccctgctgctgggcttcagctggctcctgctggtggccatcctgttctgggtgctctggctggtcatcctcattctgctgctgctggtgtaccctattcctcaccaccccctgcccacctccctcaggtttagaatgaagcagagggtgagcagcgaccccacaggttctgacagaagccctcagggcagccataatagcctgaactcccccgatgaggaggaccccaaggatgacaccaagcaacctctgtgcaacatgacccagggcggacctcccgtcaatggacagctcctcggacaacatgctcaatgcccccctcactatccctgctgccatattcagcatcccgacggagaggattccgatggagacgatggcaagtcctggggcgatgccggagaggaagacaatggccctaacgaccctaacaccgccagcaccagagagtccatttacgaggacctcagataccccacaagggacgccaatggcgagtatgagaacgtgggatacccccctagggacggagatgcccctcataggctcggagagcctgtgtatgacgatgtggagcaagccaccgctaacgaggtgagaatctcccctctgttcagactgccctacggaagcgctttcggacctggcccccagcctggacccattctggagagctccacatggggctttctggtcttcacacagacctccctgttcgccgacgacattgccgacgctattagggactactgcacaacccaccctggccccacaaggaacacccaggtggtcctcatgaacttcgagggcagcggagtgcccctgcctatgttttttccccctggagaggagacagaagagcagagagagggcgatagagctagcgactccgacgagtccgaagacgctcagatcctgaccgtgttctgcctgttttgccagtggacactctttatctgcctgggaatcaggatgatctgtaactggaggggcaaactcaccaggatcatctgcctgaagttctgcctctacggactgatttccgcctccctgtccttcggctggtacgcttttctgaaggaagtgaccctccccaccacagccaccgttgatcctaggcaactccccctgttcctcttcatcctgagctccgtgctggtgattctcgccatcatgatggagtttcaaacatcctccagcctcttcgctgctctgttcgtgattatcgccggaatgctgtgcgtcacagtgggcgtgatttttctgctggctggcgtcaagcctctcctgagcggcatgatctgcgcctccggcatcacaatgctcgtgctcggcgtcgtgctgctggtggtgtgcaccagaagccccagcccttgtcatcacagggatgaacccccctccagaagccccagccctcaacccaccgtctccgagcagtcccagcagtcccccaggcagcagagccctcaaggcacatcccagggttctacaagacctcaggtgcctggaggcgccaccaccagaaaaagaggcggcgtgagaggccaacctgccaagtgtcacggcaagtacaccacaaccgccgagggactgaccgctctcctgaataggaggcacagccccaggacatccaacgagggcaggtggatgaatggagtcatggctgtgaacctctccaaatggcccctgtacagcctgaggagagccctggccctcgccatggctcctagaaggaggctctccggccctccctggctgacagtgctgctgctgctgtccacactgagcgtggccgccctgctgattctcttcctgattttcagcgccggcgccaccattagcacagaagccagcctgctggtcctgctcctgctgtttgtgaccctgctgctgcctctcctgtcctccaacggactccagctccctgccgccctgattctgatccagtgtttcctcctggccgctgattatctcgcctacctgattctgcctaccattatgcccaggggcagaagcacaggaaggaagggcagggacacagagaaagagaggagcagatcccctctcagagctcctggcggttctgatggacccagcacaagggctggctgtggagccggaccctgtcagctgagcagccccatcgccggaaacaacggcaatgaaggcggcgagggcgacgactacaagagctggaggaagcccgaggaagaggacaacggccccaatgaccccaataccaacaacaggattgaggatggagacggcgacgacggaaaatcctggaggaatcctgaggaggaggataacagaaagcaggacaggctgggcaccaagcctttcatggccggccactggtatgagagcgtgattcccggcctgttcctctgccccctgatcctcccttccctgttctggatttgctccctgctgaccttcctggtgggccacggagccaatattgtgagcgccgtcctgttcctcgtgctggcttggtgtctcctcattgccaactggaacgtgacaagagaggacttcgtgtccggcaggagaagctccatgagcagcctgtccgtggccgcttccaccgccacagccatgttcgccagcttcctcaccctgagctttgatggcctgggcctgctgctgtttggcaccgccctggtgatccagacaatttacgtgctgtatctggtggtcatggagatcaccgtgtggatcatgatgtttaggtatctccacttttggatcaccctgctgttcctgctgagccccattattctctccgtcgcctgtctcatcatccaatcctccgccctgctgatcgaggctgtggtcgtcaccaccatcacagtcctggccatttttctgtggctccctcctcaaggcgctgaggccgatctcggcaccgccctgctgattctgaataccgccctgtgcctggtcgtgctgatcctgaccgctatccctacatgatgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaangcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactggggctcctgtcgccatcctgtgaacgccaccgtcttcacccaccccgaccaggcccaggcgaacctcacctgcggtctgcatcggagggccaagaagtacctcacctggtacttcaacggcaccccctttgtggtttacaacagcttcgacggggacggagtctccctgaaagaccagctctccggtctcagctactccatccacaagaacaccaccctccaactcttccctccctacctgccgggaacctacgagtgcgtcaccggccgctgcacccacctcacccgcctgatcgtaaaccagagctttccgggaacagataactccctcttccccagaacaggaggtgagctcaggaaactccccggggaccagggcggagacgtaccttcgacccttgtggggttaggattttttattaccgggttgctggctcttttaatcaaagtttccttgagatttgttctttccttctacgtgtatgaacacctcaacctccaataactctaccctttcttcggaatcaggtgacttctctgaaatcgggcttggtgtgctgcttactctgttgatttttttccttatcatactcagccttctgtgcctcaggctcgccgcctgctgcgcacacatctatatctactgctggttgctcaagtgcaggggtcgccacccaagatgaacaggtacatggtcctatcgatcctaggcctgctggccctggcggcctgcagcgccgccaaaaaagagattacctttgaggagcccgcttgcaatgtaactttcaagcccgagggtgaccaatgcaccaccctcgtcaaatgcgttaccaatcatgagaggctgcgcatcgactacaaaaacaaaactggccagtttgcggtctatagtgtgtttacgcccggagacccctctaactactctgtcaccgtcttccagggcggacagtctaagatattcaattacactttccctttttatgagttatgcgatgcggtcatgtacatgtcaaaacagtacaacctgtggcctccctctccccaggcgtgtgtggaaaatactgggtcttactgctgtatggctttcgcaatcactacgctcgctctaatctgcacggtgctatacataaaattcaggcagaggcgaatctttatcgatgaaaagaaaatgccttgatcgctaacaccggctttctatctgcagaatgaatgcaatcacctccctactaatcaccaccaccctccttgcgattgcccatgggttgacacgaatcgaagtgccagtggggtccaatgtcaccatggtgggccccgccggcaattccaccctcatgtgggaaaaatttgtccgcaatcaatgggttcatttctgctctaaccgaatcagtatcaagcccagagccatctgcgatgggcaaaatctaactctgatcaatgtgcaaatgatggatgctgggtactattacgggcagcggggagaaatcattaattactggcgaccccacaaggactacatgctgcatgtagtcgaggcacttcccactaccacccccactaccacctctcccaccaccaccaccactactactactactactactactactactactaccactaccgctgcccgccatacccgcaaaagcaccatgattagcacaaagccccctcgtgctcactcccacgccggcgggcccatcggtgcgacctcagaaaccaccgagctttgcttctgccaatgcactaacgccagcgctcatgaactgttcgacctggagaatgaggatgtccagcagagctccgcttgcctgacccaggaggctgtggagcccgttgccctgaagcagatcggtgattcaataattgactcttcttcttttgccactcccgaataccctcccgattctactttccacatcacgggtaccaaagaccctaacctctctttctacctgatgctgctgctctgtatctctgtggtctcttccgcgctgatgttactggggatgttctgctgcctgatctgccgcagaaagagaaaagctcgctctcagggccaaccactgatgcccttcccctaccccccggattttgcagataacaagatatgagctcgctgctgacactaaccgctttactagcctgcgctctaacccttgtcgcttgcgactcgagattccacaatgtcacagctgtggcaggagaaaatgttactttcaactccacggccgatacccagtggtcgtggagtggctcaggtagctacttaactatctgcaatagctccacttcccccggcatatccccaaccaagtaccaatgcaatgccagcctgttcaccctcatcaacgcttccaccctggacaatggactctatgtaggctatgtaccctttggtgggcaaggaaagacccacgcttacaacctggaagttcgccagcccagaaccactacccaagcttctcccaccaccaccaccaccaccaccatcaccagcagcagcagcagcagcagccacagcagcagcagcagattattgactttggttttggccagctcatctgccgctacccaggccatctacagctctgtgcccgaaaccactcagatccaccgcccagaaacgaccaccgccaccaccctacacacctccagcgatcagatgccgaccaacatcacccccttggctcttcaaatgggacttacaagccccactccaaaaccagtggatgcggccgaggtctccgccctcgtcaatgactgggcggggctgggaatgtggtggttcgccataggcatgatggcgctctgcctgcttctgctctggctcatctgctgcctccaccgcaggcgagccagaccccccatctatagacccatcattgtcctgaaccccgataatgatgggatccatagattggatggcctgaaaaacctacttttttcttttacagtatgataaattgagacatgcctcgcattttcttgtacatgttccttctcccaccttttctggggtgttctacgctggccgctgtgtctcacctggaggtagactgcctctcacccttcactgtctacctgctttacggattggtcaccctcactctcatctgcagcctaatcacagtaatcatcgccttcatccagtgcattgattacatctgtgtgcgcctcgcatacttcagacaccacccgcagtaccgagacaggaacattgcccaacttctaagactgctctaatcatgcataagactgtgatctgccttctgatcctctgcatcctgcccaccctcacctcctgccagtacaccacaaaatctccgcgcaaaagacatgcctcctgccgcttcacccaactgtggaatatacccaaatgctacaacgaaaagagcgagctctccgaagcttggctgtatggggtcatctgtgtcttagttttctgcagcactgtctttgccctcataatctacccctactttgatttgggatggaacgcgatcgatgccatgaattaccccacctttcccgcacccgagataattccactgcgacaagttgtacccgttgtcgttaatcaacgccccccatcccctacgcccactgaaatcagctactttaacctaacaggcggagatgactgacgccctagatctagaaatggacggcatcagtaccgagcagcgtctcctagagaggcgcaggcaggcggctgagcaagagcgcctcaatcaggagctccgagatctcgttaacctgcaccagtgcaaaagaggcatcttttgtctggtaaagcaggccaaagtcacctacgagaagaccggcaacagccaccgcctcagttacaaattgcccacccagcgccagaagctggtgctcatggtgggtgagaatcccatcaccgtcacccagcactcggtagagaccgaggggtgtctgcactccccctgtcggggtccagaagacctctgcaccctggtaaagaccctgtgcggtctcagagatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatggtttaaacggatcctctagagtcgacctgcaggcatgcaagcttgagtataacccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgctcggtacccggggatcctcgtttaaac

SEQ ID NO: 53 - DNA, 41545 bp, synthetic construct
  misc_difference 16144: note = modified_base - a, c, t, g, unknown or other
  misc_feature 1..41545: note = Description of Artificial Sequence: Synthetic polynucleotide
  source 1..41545: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatgtccgaggactttctgattctgatcgccatcctggtgatcgtgattctcgtgggcacaatcacaaccctggtgggcgccatcggcggcattagggccaggaggagcttcctcttcatttgcatcttcttcctgttcctctccctcttcctgacaatcctcgccctgctgctgggcttcagctggctcctgctggtggccatcctgttctgggtgctctggctggtcatcctcattctgctgctgctggtgtaccctattcctcaccaccccctgcccacctccctcaggtttagaatgaagcagagggtgagcagcgaccccacaggttctgacagaagccctcagggcagccataatagcctgaactcccccgatgaggaggaccccaaggatgacaccaagcaacctctgtgcaacatgacccagggcggacctcccgtcaatggacagctcctcggacaacatgctcaatgcccccctcactatccctgctgccatattcagcatcccgacggagaggattccgatggagacgatggcaagtcctggggcgatgccggagaggaagacaatggccctaacgaccctaacaccgccagcaccagagagtccatttacgaggacctcagataccccacaagggacgccaatggcgagtatgagaacgtgggatacccccctagggacggagatgcccctcataggctcggagagcctgtgtatgacgatgtggagcaagccaccgctaacgaggtgagaatctcccctctgttcagactgccctacggaagcgctttcggacctggcccccagcctggacccattctggagagctccacatggggctttctggtcttcacacagacctccctgttcgccgacgacattgccgacgctattagggactactgcacaacccaccctggccccacaaggaacacccaggtggtcctcatgaacttcgagggcagcggagtgcccctgcctatgttttttccccctggagaggagacagaagagcagagagagggcgatagagctagcgactccgacgagtccgaagacgctcagatcctgaccgtgttctgcctgttttgccagtggacactctttatctgcctgggaatcaggatgatctgtaactggaggggcaaactcaccaggatcatctgcctgaagttctgcctctacggactgatttccgcctccctgtccttcggctggtacgcttttctgaaggaagtgaccctccccaccacagccaccgttgatcctaggcaactccccctgttcctcttcatcctgagctccgtgctggtgattctcgccatcatgatggagtttcaaacatcctccagcctcttcgctgctctgttcgtgattatcgccggaatgctgtgcgtcacagtgggcgtgatttttctgctggctggcgtcaagcctctcctgagcggcatgatctgcgcctccggcatcacaatgctcgtgctcggcgtcgtgctgctggtggtgtgcaccagagatgagcacgctatttccgccagccaccatgctagcgatggctccgtgaatcagcagaaggaaaatcagccccagaccctggaggaatgcaagacagatcaggagaggaagaggtacaggaacaggctggcctccaggaggtgtagagctaagttcaggaaccagctggaacattttaggacagtcgccgctgctaagacagaggagaacaacaggctcagggtgctcatcaggcagatgtgtcctacactggacgtggaatccatcgtcccctccacctccgccggctaccacgagcctctgaatcacctgacccacagccccagcccttgtcatcacagggatgaacccccctccagaagccccagccctcaacccaccgtctccgagcagtcccagcagtcccccaggcagcagagccctcaaggcacatcccagggttctacaagacctcaggtgcctggaggcgccaccaccagaaaaagaggcggcgtgagaggccaacctgccaagtgtcacggcaagtacaccacaaccgccgagggactgaccgctctcctgaataggaggcacagccccaggacatccaacgagggcaggtggatgaatggagtcatggctgtgaacctctccaaatggcccctgtacagcctgaggagagccctggccctcgccatggctcctagaaggaggctctccggccctccctggctgacagtgctgctgctgctgtccacactgagcgtggccgccctgctgattctcttcctgattttcagcgccggcgccaccattagcacagaagccagcctgctggtcctgctcctgctgtttgtgaccctgctgctgcctctcctgtcctccaacggactccagctccctgccgccctgattctgatccagtgtttcctcctggccgctgattatctcgcctacctgattctgcctaccattatgcccaggggcagaagcacaggaaggaagggcagggacacagagaaagagaggagcagatcccctctcagagctcctggcggttctgatggacccagcacaagggctggctgtggagccggaccctgtcagctgagcagccccatcgccggaaacaacggcaatgaaggcggcgagggcgacgactacaagagctggaggaagcccgaggaagaggacaacggccccaatgaccccaataccaacaacaggattgaggatggagacggcgacgacggaaaatcctggaggaatcctgaggaggaggataacagaaagcaggacaggctgggcaccaagcctttcatggacctcgacggaaccggcggaggcgagggctacagccagatggtccctatcgccaccgcccccggaagcggccacgccgctacctatcaggatctccaggccgccccttacatcatctggcctctccagaccgattgccagcctgtggctaccaccttcgcctcccccggacagatccagtggtatacaagcgccgtcccccagcccacagagcattgctcccagtttacaaacgctcccaccgtcaaccagcagcagcctattagccaaccccagcccgaaaatccccctgctttcacctttacccagcccgcttccatcattcccggcgtcattagcgcctccaacctgaacgtgagcgcttcccctatcatccctagcgaccatgtcctccccatcattacctccgtgaccagcctcgcccaacctaataacatggccggccactggtatgagagcgtgattcccggcctgttcctctgccccctgatcctcccttccctgttctggatttgctccctgctgaccttcctggtgggccacggagccaatattgtgagcgccgtcctgttcctcgtgctggcttggtgtctcctcattgccaactggaacgtgacaagagaggacttcgtgtccggcaggagaagctccatgagcagcctgtccgtggccgcttccaccgccacagccatgttcgccagcttcctcaccctgagctttgatggcctgggcctgctgctgtttggcaccgccctggtgatccagacaatttacgtgctgtatctggtggtcatggagatcaccgtgtggatcatgatgtttaggtatctccacttttggatcaccctgctgttcctgctgagccccattattctctccgtcgcctgtctcatcatccaatcctccgccctgctgatcgaggctgtggtcgtcaccaccatcacagtcctggccatttttctgtggctccctcctcaaggcgctgaggccgatctcggcaccgccctgctgattctgaataccgccctgtgcctggtcgtgctgatcctgaccgctatccctacatgatgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaangcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactgggatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatggtttaaacggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctcgtttaaac

SEQ ID NO: 54 - DNA, 42220 bp, synthetic construct
  misc_difference 16789: note = modified_base - a, c, t, g, unknown or other
  misc_feature 1..42220: note = Description of Artificial Sequence: Synthetic polynucleotide
  source 1..42220: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatggacgaccagcgggacctgatcagcaacaacgagcagctgcccatgctgggccagaggcctggcgcccctgagagcaagtgtagcagaggcgccgtgtacaccgtgttcagcatcctggtggccctgctgctggccggacaggccaccaccgcctactttctgtatcagcagcagggacggctggacaagctgaccgtgaccagccagaacctgcagctggaaaacctgcggatgaagctgcccaagcccgccaagcccctgagccagatgagaatggccacccccctgctgatgcaggccctgcctatggccggcctgccccagaaacccatgcagaacgccaccaagcacggcaacatgaccgaggaccacgtgatgcatctgctgctgaacgccgaccccctgaaggtgtaccccccactgaagggcagcctgagcgagaacctgaagcacctgaagaacaccatggaaaccatggactggaaggtgttcgagagctggctgcaccactggctgctgttcgagatgagcaagcacagcctggaacagaagcccaccgaggcccctcccaaagagagcctggaactggaagatcccagcagcggcctgggcgtgaccaagcaggatctgggccccgtggctatgtccgaggactttctgattctgatcgccatcctggtgatcgtgattctcgtgggcacaatcacaaccctggtgggcgccatcggcggcattagggccaggaggagcttcctcttcatttgcatcttcttcctgttcctctccctcttcctgacaatcctcgccctgctgctgggcttcagctggctcctgctggtggccatcctgttctgggtgctctggctggtcatcctcattctgctgctgctggtgtaccctattcctcaccaccccctgcccacctccctcaggtttagaatgaagcagagggtgagcagcgaccccacaggttctgacagaagccctcagggcagccataatagcctgaactcccccgatgaggaggaccccaaggatgacaccaagcaacctctgtgcaacatgacccagggcggacctcccgtcaatggacagctcctcggacaacatgctcaatgcccccctcactatccctgctgccatattcagcatcccgacggagaggattccgatggagacgatggcaagtcctggggcgatgccggagaggaagacaatggccctaacgaccctaacaccgccagcaccagagagtccatttacgaggacctcagataccccacaagggacgccaatggcgagtatgagaacgtgggatacccccctagggacggagatgcccctcataggctcggagagcctgtgtatgacgatgtggagcaagccaccgctaacgaggtgagaatctcccctctgttcagactgccctacggaagcgctttcggacctggcccccagcctggacccattctggagagctccacatggggctttctggtcttcacacagacctccctgttcgccgacgacattgccgacgctattagggactactgcacaacccaccctggccccacaaggaacacccaggtggtcctcatgaacttcgagggcagcggagtgcccctgcctatgttttttccccctggagaggagacagaagagcagagagagggcgatagagctagcgactccgacgagtccgaagacgctcagatcctgaccgtgttctgcctgttttgccagtggacactctttatctgcctgggaatcaggatgatctgtaactggaggggcaaactcaccaggatcatctgcctgaagttctgcctctacggactgatttccgcctccctgtccttcggctggtacgcttttctgaaggaagtgaccctccccaccacagccaccgttgatcctaggcaactccccctgttcctcttcatcctgagctccgtgctggtgattctcgccatcatgatggagtttcaaacatcctccagcctcttcgctgctctgttcgtgattatcgccggaatgctgtgcgtcacagtgggcgtgatttttctgctggctggcgtcaagcctctcctgagcggcatgatctgcgcctccggcatcacaatgctcgtgctcggcgtcgtgctgctggtggtgtgcaccagagatgagcacgctatttccgccagccaccatgctagcgatggctccgtgaatcagcagaaggaaaatcagccccagaccctggaggaatgcaagacagatcaggagaggaagaggtacaggaacaggctggcctccaggaggtgtagagctaagttcaggaaccagctggaacattttaggacagtcgccgctgctaagacagaggagaacaacaggctcagggtgctcatcaggcagatgtgtcctacactggacgtggaatccatcgtcccctccacctccgccggctaccacgagcctctgaatcacctgacccacagccccagcccttgtcatcacagggatgaacccccctccagaagccccagccctcaacccaccgtctccgagcagtcccagcagtcccccaggcagcagagccctcaaggcacatcccagggttctacaagacctcaggtgcctggaggcgccaccaccagaaaaagaggcggcgtgagaggccaacctgccaagtgtcacggcaagtacaccacaaccgccgagggactgaccgctctcctgaataggaggcacagccccaggacatccaacgagggcaggtggatgaatggagtcatggctgtgaacctctccaaatggcccctgtacagcctgaggagagccctggccctcgccatggctcctagaaggaggctctccggccctccctggctgacagtgctgctgctgctgtccacactgagcgtggccgccctgctgattctcttcctgattttcagcgccggcgccaccattagcacagaagccagcctgctggtcctgctcctgctgtttgtgaccctgctgctgcctctcctgtcctccaacggactccagctccctgccgccctgattctgatccagtgtttcctcctggccgctgattatctcgcctacctgattctgcctaccattatgcccaggggcagaagcacaggaaggaagggcagggacacagagaaagagaggagcagatcccctctcagagctcctggcggttctgatggacccagcacaagggctggctgtggagccggaccctgtcagctgagcagccccatcgccggaaacaacggcaatgaaggcggcgagggcgacgactacaagagctggaggaagcccgaggaagaggacaacggccccaatgaccccaataccaacaacaggattgaggatggagacggcgacgacggaaaatcctggaggaatcctgaggaggaggataacagaaagcaggacaggctgggcaccaagcctttcatggacctcgacggaaccggcggaggcgagggctacagccagatggtccctatcgccaccgcccccggaagcggccacgccgctacctatcaggatctccaggccgccccttacatcatctggcctctccagaccgattgccagcctgtggctaccaccttcgcctcccccggacagatccagtggtatacaagcgccgtcccccagcccacagagcattgctcccagtttacaaacgctcccaccgtcaaccagcagcagcctattagccaaccccagcccgaaaatccccctgctttcacctttacccagcccgcttccatcattcccggcgtcattagcgcctccaacctgaacgtgagcgcttcccctatcatccctagcgaccatgtcctccccatcattacctccgtgaccagcctcgcccaacctaataacatggccggccactggtatgagagcgtgattcccggcctgttcctctgccccctgatcctcccttccctgttctggatttgctccctgctgaccttcctggtgggccacggagccaatattgtgagcgccgtcctgttcctcgtgctggcttggtgtctcctcattgccaactggaacgtgacaagagaggacttcgtgtccggcaggagaagctccatgagcagcctgtccgtggccgcttccaccgccacagccatgttcgccagcttcctcaccctgagctttgatggcctgggcctgctgctgtttggcaccgccctggtgatccagacaatttacgtgctgtatctggtggtcatggagatcaccgtgtggatcatgatgtttaggtatctccacttttggatcaccctgctgttcctgctgagccccattattctctccgtcgcctgtctcatcatccaatcctccgccctgctgatcgaggctgtggtcgtcaccaccatcacagtcctggccatttttctgtggctccctcctcaaggcgctgaggccgatctcggcaccgccctgctgattctgaataccgccctgtgcctggtcgtgctgatcctgaccgctatccctacatgatgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaangcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactgggatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatggtttaaacggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcagctcggtacccggggatcctcgtttaaacaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctcgtttaaac